(19) United States
(12) Patent Application Publication
(10) Pub. No.: US 2013/0267429 A1
(43) Pub. Date: Oct. 10, 2013

(54) BIOLOGICAL SAMPLE TARGET CLASSIFICATION, DETECTION AND SELECTION METHODS, AND RELATED ARRAYS AND OLIGONUCLEOTIDE PROBES

(71) Applicant: Lawrence Livermore National Security, LLC, Livermore, CA (US)

(72) Inventors: Shea GARDNER, Oakland, CA (US); Crystal J. JAING, Livermore, CA (US); Kevin MCLOUGHLIN, Oakland, CA (US); Thomas SLEZAK, San Francisco, CA (US); James B. THISSEN, Alameda, CA (US); Marisa Wailam TORRES, Pleasanton, CA (US)

(21) Appl. No.: 13/886,172

(22) Filed: May 2, 2013

Related U.S. Application Data
(63) Continuation-in-part of application No. 13/304,276, filed on Nov. 23, 2011.
Application No. 13/304,276 is a continuation-in-part of application No. 12/643,903, filed on Dec. 21, 2009.
(60) Provisional application No. 61/628,224, filed on Oct. 26, 2011.

(51) Int. Cl. G06F 19/20; C12Q 1/68

(52) U.S. Cl. CPC G06F 19/20; C12Q 1/6876
USPC 506/8; 506/16

(57) ABSTRACT

Biological sample target classification, detection and selection methods are described, together with related arrays and oligonucleotide probes.

### CROSS REFERENCE TO RELATED APPLICATIONS

This application is a continuation in part of U.S. application Ser. No. 13/304,276 entitled “Biological Sample Target Classification, Detection and Selection Methods, and Related Arrays and Oligonucleotide Probes” filed on Nov. 23, 2011 which is, in turn, a continuation in part of U.S. application Ser. No. 12/643,903 entitled “Biological Sample Target Classification, Detection and Selection Methods, and Related Arrays and Oligonucleotide Probes” filed on Dec. 21, 2009 and claims priority to U.S. provisional application No. 61/628,224 filed on Oct. 26, 2011, each of which is incorporated herein by reference in its entirety.

### STATEMENT OF GOVERNMENT GRANT

The United States Government has rights in this invention pursuant to Contract No. DE-AC52-07NA27344 between the U.S. Department of Energy and Lawrence Livermore National Security, LLC, for the operation of Lawrence Livermore National Security.

### FIELD

The present disclosure relates to arrays, methods and systems for pan microbial detection. In particular, the present disclosure relates to biological sample target classification, detection and selection methods, and related arrays and oligonucleotide probes.

### BACKGROUND

Various approaches for detecting microbial presence are based on use of arrays and in particular, probe microarrays.

Microarrays can be used for microbial surveillance, detection and discovery. These arrays probe species-specific or conserved regions to enable detection of novel organisms with some homology to the probes designed from sequenced organisms. Detection microarrays have proven useful in identifying, subtyping, or discovering viruses with homology to known viruses (see references 4, 10, 11, 15, 16, 18, 21, 23, 24 and 25).

Bacterial detection arrays to date have focused on highly conserved rRNA regions (16S or 23S) (see references 1, 5, 9, 14, 24) allowing specific rather than random PCR to amplify the target region with highly conserved primers. Virus diversity precludes the identification of a particular gene universally conserved at the nucleotide level for viruses, and viral probe design requires consideration of many genes or whole genomes.

The ViroChip discovery array played a role in characterizing SARS as a coronavirus (see references 16, 22 and 23). It was built using techniques for selecting probes from regions of conservation based on BLAST nucleotide sequence similarity to viruses in the respective viral family, such that all viruses sequenced at the time of design (2004) would be represented by 5-10 probes. Version 3 of the Virochip included approximately 22,000 probes. Chou et al. (see reference 4) designed conserved genus probes and species specific probes covering 53 viral families and 214 genera, requiring 2 probes per virus.

### SUMMARY

Provided herein in accordance with several embodiments of the present disclosure are biological sample target classification, detection and selection methods, and related arrays and oligonucleotide probes.

According to a first aspect, a method to obtain a plurality of oligonucleotide probes for detection of targets of a target group is provided, comprising: identifying group-specific candidate probes from an initial genomic collection by eliminating from the initial collection regions with matches to non-group targets above a match threshold and by selecting regions satisfying probe characteristics, said probe characteristics including at least one criterion selected from length, Tₘ, GC %, maximum homopolymer length, homodimer free energy prediction, hairpin free energy prediction, probe-target free energy prediction, and minimum trimer frequency entropy condition; ranking the group-specific candidate probes in decreasing order of number of targets of the target group represented by each group-specific candidate probe; and selecting probes from the ranked group-specific candidate probes.

According to a second aspect, a method of classifying an oligonucleotide probe sequence as detected or undetected in a biological sample is provided, comprising: incubating fluorescently labeled target DNA synthesized from templates extracted from a biological sample on an array comprising a plurality of probes, to allow for hybridization of target DNA to any probes of the array having sequences similar to those of the target DNA, producing a variable number of target-probe hybridization products for each probe sequence; scanning the array to measure an aggregate fluorescence intensity value for each feature comprising a set of target-probe hybridization products having probes of the same sequence; calculating the distribution of feature intensity values for target-probe hybridization products by way of negative control probes with randomly generated sequences, and setting a minimum detection threshold for the array; and comparing the observed feature intensity value for each probe sequence with the minimum detection threshold determined for the array, to classify each probe sequence on the array as either detected or undetected in the biological sample.

According to a third aspect, a method of predicting likelihood of presence of a target of known nucleotide sequence in a biological sample is provided, comprising: applying the method according to the above second aspect to classify probe sequences on an array as detected or undetected in the sample; estimating, for each detected probe sequence: i) a probability of observing the probe sequence as detected conditioned on presence of the target of known nucleotide sequence; ii) a probability of observing the probe sequence as detected conditioned on absence of the target of known nucleotide sequence; and iii) the detection log-odds, defined as the ratio of i) and ii); estimating, for each undetected probe sequence: iv) a probability of observing the probe sequence as undetected conditioned on presence of the target of known nucleotide sequence; v) a probability of observing the probe sequence as undetected conditioned on absence of the target of known nucleotide sequence; and vi) the nondetection log-odds, defined as the ratio of iv) and v); summing detection and nondetection log-odds values over the probes on the array to form an aggregate log-odds score for presence versus absence of the target of known nucleotide sequence, conditional on the observed detected and undetected probes; and based on the aggregate log-odds score, providing a prediction of the presence of at least one said target of known nucleotide sequence in the biological sample.

According to a fourth aspect, a selection method for selecting, from a list of candidate target sequences of known nucleotide sequence, a target sequence most likely to be present in a biological sample is provided, the selection method comprising: applying the method according to the above third aspect to each of the candidate target sequences, and choosing the target sequence that yields the maximum aggregate log-odds score.

According to a fifth aspect, a selection method for selecting, from a list of candidates, a set of targets whose presence in a biological sample would collectively provide the best explanation for observed detected and undetected probes on an array is provided, comprising: a) applying the above method to identify the target most likely to be present in the sample; b) removing the identified target from the list of candidates and adding the identified target to the “selected” list; c) repeating the method of claim 17 for the remaining candidates, wherein: c1) estimation of i), ii) and iii) is replaced with estimation of: i′) a probability of observing the probe sequence as detected conditioned on presence of the candidate target and presence of targets in the list of selected targets; ii′) a probability of observing the probe sequence as detected conditioned on absence of the candidate target and presence of targets in the list of selected targets; and iii′) the detection log-odds, defined as the ratio of i′) and ii′); c2) estimation of iv), v) and vi) is replaced with estimation of: iv′) a probability of observing the probe sequence as undetected conditioned on presence of the candidate target and presence of targets in the list of selected targets; v′) a probability of observing the probe sequence as undetected conditioned on absence of the candidate target and presence of the targets in the list of selected targets; and vi′) the nondetection log-odds, defined as the ratio of iv′) and v′); c3) the detection and nondetection log-odds values are summed over the probes on the array to form a conditional log-odds score for presence versus absence of the candidate target, conditioned on the observed detected and undetected probes and on the presence of the targets in the list of selected targets; d) choosing the candidate target yielding the maximum conditional log-odds score, removing it from the candidate list, and adding it to the list of selected targets; and e) repeating c) and d) until the conditional log-odds scores for all remaining candidate targets are less than zero.

According to a sixth aspect, an oligonucleotide probe for detection of targets in a target group is described, the oligonucleotide probe comprising a sequence selected from the group consisting of SEQ ID NO's 1-133,263, wherein: said detection occurs in combination with other oligonucleotide probes selected from the group consisting of SEQ ID NO's 1-133,263, and said target is a microorganism. In particular, the detection can be performed in combination with at least four other oligonucleotide probes selected from the group consisting of SEQ ID NO's 1-133,263.

According to a seventh aspect, a system for detection of at least one target in a target group is described, the system comprising at least two oligonucleotide probes, wherein: each oligonucleotide probe comprises a sequence selected from the group consisting of SEQ ID NO's 1-133,263, wherein the at least one target is a microorganism and wherein the detection occurs in combination with other oligonucleotide probes selected from the group consisting of SEQ ID NO's 1-133,263. In particular, the detection can be performed in combination with at least other three other oligonucleotide probes selected from the group consisting of SEQ ID NO's 1-133,263.

According to an eighth aspect, an array for detection of targets in a target group, is described, the array comprising a plurality of oligonucleotide probes wherein: at least one of the oligonucleotide probes comprises a sequence selected from the group consisting of SEQ ID NO. 1 to SEQ ID NO: 133,263; the detection occurs in combination with other oligonucleotide probes selected from the group consisting of SEQ ID NO's 1 to SEQ ID NO: 133,263, and wherein said target is a microorganism. In particular, the detection can be performed in combination with at least four other oligonucleotide probes selected from the group consisting of SEQ ID NO's 1 to SEQ ID NO: 133,263.

According to a ninth aspect, a computer-based method to obtain a plurality of oligonucleotide probes for detection of targets of a target group is provided. The computer based method comprises computer-operated steps, where a computer performs the steps in single-processor mode or multiple-processor mode. The computer operated steps comprises providing an initial genomic collection, identifying group-specific candidate probes from the initial genomic collection by eliminating from the initial collection regions with matches to non-group targets above a match threshold and by selecting regions satisfying probe characteristics, said probe characteristics including at least one criterion selected from length, Tm, GC %, maximum homopolymer length, homodimer free energy prediction, hairpin free energy prediction, probe-target free energy prediction, and minimum trimer frequency entropy condition, ranking the group-specific candidate probes in decreasing order of number of targets of the target group represented by each group-specific candidate probe, and selecting probes from the ranked group-specific candidate probes, thus obtaining the plurality of oligonucleotide probes for detection of targets of a target group, where a target is represented if a candidate probe matches with at least 85% sequence similarity over the total candidate probe length and has a perfectly matching subsequence of at least 29 contiguous bases spanning the middle of the probe.

According to a tenth aspect, a computer-based method to obtain a plurality of oligonucleotide probes for detection of targets of a target group is provided. The computer based method comprises computer-operated steps where a computer performs the steps in single-processor mode or multiple-processor mode. The computer operated steps comprises providing an initial genomic collection, identifying group-specific candidate probes from the initial genomic collection by eliminating from the initial collection regions with matches to non-group targets above a match threshold and by selecting regions satisfying probe characteristics, said probe characteristics including at least one criterion selected from length, Tm, GC %, maximum homopolymer length, homodimer free energy prediction, hairpin free energy prediction, probe-target free energy prediction, and minimum trimer frequency entropy condition, ranking the group-specific candidate probes in decreasing order of number of targets of the target group represented by each group-specific candidate probe, selecting probes from the ranked group-specific candidate probes, thus obtaining the plurality of oligonucleotide probes for detection of targets of a target group, where a target is represented if a candidate probe matches an at least 85% sequence identity to the target over the length of the probe and a detection probability of at least 85% derived from an alignment score, a predicted Tm, and the start position of the match on the probe.

According to an eleventh aspect, a computer-based method to obtain a plurality of oligonucleotide probes for detection of targets of a target group is provided. The computer based method comprises computer-operated steps where a computer performs the steps in single-processor mode or multiple-processor mode. The computer operated steps comprises providing an initial genomic collection, identifying group-specific candidate probes from the initial genomic collection by k-mer analysis. k-mer analysis comprises compiling sequences of targets independent of any alignment, enumerating all k-mers of a desired probe length range of the compiled sequences, where k is the desired number of bases in a family-unique region, ranking k-mers by the number of target sequences in which they occur, picking conserved k-mers from the ranked k-mers, filtering conserved k-mers for desired characteristics, aligning filtered conserved k-mers to targets, recording detected targets from the alignment as probes, where the recording is iterated to find another k-mer for remaining targets, aligning probes against target sequences, and selecting probes from the matches of the alignments that satisfy at least a minimum desired probe/oligo length, thus obtaining the plurality of oligonucleotide probes for detection of targets of a target group.

According to a twelveth aspect, an oligonucleotide probe for detection of at least one target in a target group is provided. The oligonucleotide probe comprises a sequence selected from a group consisting of SEQ ID NO's 491,463-495,658 and 534,157-661,081, where said detection occurs in combination with at least four other oligonucleotide probes selected from the group consisting of SEQ ID NO's 491,463-495,658 and 534,157-661,081; and said target is a microorganism.

According to a thirteenth aspect, a system for detection of at least one target in a target group is provided. The system comprises at least five oligonucleotide probes, where each oligonucleotide probe comprises a sequence selected from the group consisting of SEQ ID NO's 491,463-495,658 and 534,157-661,081, and where at least one target is a microorganism.

According to a fourteenth aspect, an oligonucleotide probe for detection of at least one target in a target group is provided. The oligonucleotide probe comprises a sequence selected from a group consisting of SEQ ID NO's 141, 125-267-772 and 491,511-492,337 and 496,379-512,129, where said detection occurs in combination with at least four other oligonucleotide probes selected from the group consisting of SEQ ID NO's 141, 125-267-772 and 491,511-492,337 and 496,379-512,129, and said target is a bacterium.

According to a fifteenth aspect, an oligonucleotide probe for detection of at least one target in a target group is provided. The oligonucleotide probe comprises a sequence selected from a group consisting of SEQ ID NO's 297,256-486,081 and 492,545-495,045 and 492,545-495,045 and 515,887-534,156, where said detection occurs in combination with at least four other oligonucleotide probes selected from the group consisting of SEQ ID NO's 297,256-486,081 and 492,545-495,045 and 492,545-495,045 and 515,887-534,156; and said target is a virus.

According to a sixteenth aspect, an oligonucleotide probe for detection of at least one target in a target group is provided. The oligonucleotide probe comprises a sequence selected from a group consisting of SEQ ID NO's 286,566-297,255 and 492,437-492,544 and 514,810-515,886, where said detection occurs in combination with at least four other oligonucleotide probes selected from the group consisting of SEQ ID NO's 286,566-297,255 and 492,437-492,544 and 514,810-515,886, and said target is a species of protozoa.

According to a seventeenth aspect, an oligonucleotide probe for detection of at least one target in a target group is provided. The oligonucleotide probe comprises a sequence selected from a group consisting of SEQ ID NO's 133,264-141,123 and 491,463-491,510 and 495,659-496,378; where said detection occurs in combination with at least four other oligonucleotide probes selected from the group consisting of SEQ ID NO's 133,264-141,123 and 491,463-491,510 and 495,659-496,378, and said target is an archaeon.

According to an eighteenth aspect, an oligonucleotide probe for detection of at least one target in a target group is provided. The oligonucleotide probe comprises a sequence selected from a group consisting of SEQ ID NO's 267,773-286,565 and 492,338-492,436 and 512,130-514,809, where said detection occurs in combination with at least four other oligonucleotide probes selected from the group consisting of SEQ ID NO's 267,773-286,565 and 492,338-492,436 and 512,130-514,809, and said target is a fungus.

According to a nineteenth aspect, an array for detection of targets in a target group is provided. The array comprises a plurality of oligonucleotide probes where at least one of the oligonucleotide probes comprises a sequence selected from a group consisting of 491,463-495,658 and 534,157-661,081. In the array for detection of targets, the detection occurs in combination with at least four other oligonucleotide probes selected from the group consisting of 491,463-495,658 and 534,157-661,081, and where said target is a microorganism.

The methods, arrays and probes herein provided are useful for the detection of viral and bacterial sequences from single or mixed DNA and RNA viruses derived from environmental or clinical samples.

The details of one or more embodiments of the disclosure are set forth in the accompanying drawings and the detailed description and examples below. Other features, objects, and advantages will be apparent from the detailed description, examples and drawings, and from the appended claims.

### BRIEF DESCRIPTION OF THE DRAWINGS

The accompanying drawings, which are incorporated into and constitute a part of this specification, illustrate one or more embodiments of the present disclosure and, together with the detailed description and the examples, serve to explain the principles and implementations of the disclosure.

FIGS. 1A and 1B show steps of a schematic illustration of a method that is suitable to produce oligonucleotide probes for use in microbial detection arrays.

FIG. 2 shows results of an array hybridization experiment and analysis according to the disclosure. The right-hand column of bar graphs shows the unconditional and conditional log-odds scores for each target genome listed at right. That is, the darker shaded part of the bar shows the contribution from a target that cannot be explained by another, more likely target above it, while the lighter shaded part of the bar illustrates that some very similar targets share a number of probes, so that multiple targets may be consistent with the hybridization signals. The left-hand column of bar graphs shows the expectation (mean) values of the numbers of probes expected to be present given the presence of the corresponding target genome. The larger “expected” score is obtained by summing the conditional detection probabilities for all probes; the smaller “detected” score is derived by limiting this sum to probes that were actually detected. Because probes often cross-hybridize to multiple related genome sequences, the numbers of “expected” and “detected” probes often greatly exceed the number of probes that were actually designed for a given target organism.

FIGS. 3-9 show results of an array hybridization experiment and analysis similar to FIG. 2 for the indicated target genome.

FIG. 10 shows a plot of intensity distributions for adenovirus target-specific probes and negative control probes in an adenovirus limit of detection experiment at selected DNA concentrations. Hybridization was conducted for 17 hours.

FIG. 11 shows a plot of intensity distributions similar to FIG. 10 at the indicated DNA concentrations. Hybridization was conducted for 1 hour.

FIG. 12 shows distributions for an MDA v.2 array hybridized to a spiked mixture of vaccinia virus and HHV6B, for probes with and without target-specific BLAST hits and for negative control probes. Vertical line: 99^{th} percentile of negative control distribution.

FIG. 13 shows dependence of nonspecific positive signal frequency on the trimer entropy of the probe sequences. Dashed line is a logistic regression fit to the probe entropy and signal data.

FIGS. 14A and 14B show steps of an array design process diagram, illustrating the probe selection algorithm described herein.

FIG. 15 shows a schematic illustration of a method that is suitable to produce oligonucleotide probes for use in microbial detection arrays using k-mers.

FIG. 16 shows a computer system that may be used to implement the methods described.

FIG. 17 shows plots, for a particular array experiment, of the observed fraction of probes detected and the corresponding log of odds as functions of predicted detection probability and log odds.

### DETAILED DESCRIPTION

According to an embodiment of the present disclosure, methods to obtain a plurality of oligonucleotide probe sequences for detection of one or more targets within a target group are provided.

The term “oligonucleotide” as used herein refers to a polynucleotide with three or more nucleotides. In the present disclosure, oligonucleotides serve as “probes”, often when attached to and immobilized on a substrate or support. The term “polynucleotide” as used herein indicates an organic polymer composed of two or more monomers including nucleotides, nucleosides or analogs thereof. The term “nucleotide” refers to any of several compounds that consist of a ribose or deoxyribose sugar joined to a purine or pyrimidine base and to a phosphate group and that is the basic structural unit of nucleic acids. The term “nucleoside” refers to a compound (such as guanosine or adenosine) that consists of a purine or pyrimidine base combined with deoxyribose or ribose and is found especially in nucleic acids. The term “nucleotide analog” or “nucleoside analog” refers respectively to a nucleotide or nucleoside in which one or more individual atoms have been replaced with a different atom or a with a different functional group. Accordingly, the term “polynucleotide” includes nucleic acids of any length, and in particular DNA, RNA, analogs and fragments thereof.

The term “target” as used herein refers to a genomic sequence of an organism or biological particle such as a virus. Thus a “target sequence” as used herein refers to the genomic sequence of a target organism or particle. In particular, a genomic sequence includes sequences of any fully sequenced elements, nuclear (e.g. chromosome), viral segment, mitochondrial, and plasmid DNA, as well as any other nucleic acids carried by the organism or particle.

The term “target group” as used herein refers to a group of organisms or viral particles with related genomic sequences. By way of example and not of limitation, a target group can be a viral family or a bacterial family. In particular, a target family comprises the family classification according to the NCBI (National Center for Biotechnology Information) taxonomy tree. A target group can also comprise a viral, bacterial, fungal, or protozoal sequence group classified under a taxonomic node other than family.

Embodiments of the present disclosure are directed to a method to obtain a pan-Microbial Detection Array (MDA) to detect all sequenced viruses (including phage), bacteria, fungi, protozoa, archaea and plasmids and the MDA thus obtained. Family-specific probes are selected for all sequenced viral, fungal, archaea, vertebrate-infecting protozoa, and bacterial complete genomes, segments, chromosomes, mitochondrial genomes, and plasmids. In some embodiments, bacteria are those under the superkingdom Bacteria (eubacteria) taxonomy node at NCBI, and do not include the Archaea. Probes are designed to tolerate some sequence variation to enable detection of divergent species with homology to sequenced organisms. One embodiment of the array of the present disclosure (Version 3 or v3) also contains family-specific probes for all known/sequenced fungi and species-specific probes for human-infecting protozoa and their near neighbors, including probes for partial sequences (e.g. genes and other partial sequences available in collections such as the NCBI nt database). One embodiment of the array of the present disclosure (Version 5 or v5) also contains family-specific probes for all fully sequenced elements (chromosomes, plasmids, mitochondria) from archaea, fungi and vertebrate-infecting protozoa. The probes can then be arranged on suitable substrates to form an array using procedures identifiable by a skilled person upon reading of the present disclosure.

In some embodiments, fungal, bacterial, protozoan, and archaeal sequences are used and family specific sequences can be determined within each viral, bacterial, archaeal, and fungal and protozoa family and from the family specific sequences, probes can be designed to meet desired ranges for length, Tm, entropy, GC %, and other thermodynamic and sequence features In some of those embodiments, the desired ranges can be relaxed as needed to obtain at least 5 (v4) or 30 (v5) probes per sequence. Candidate probes can then be clustered and ranked by the number of targets detected, and a greedy algorithm used to select a probe set to detect as many of the targets as possible with the fewest probes.

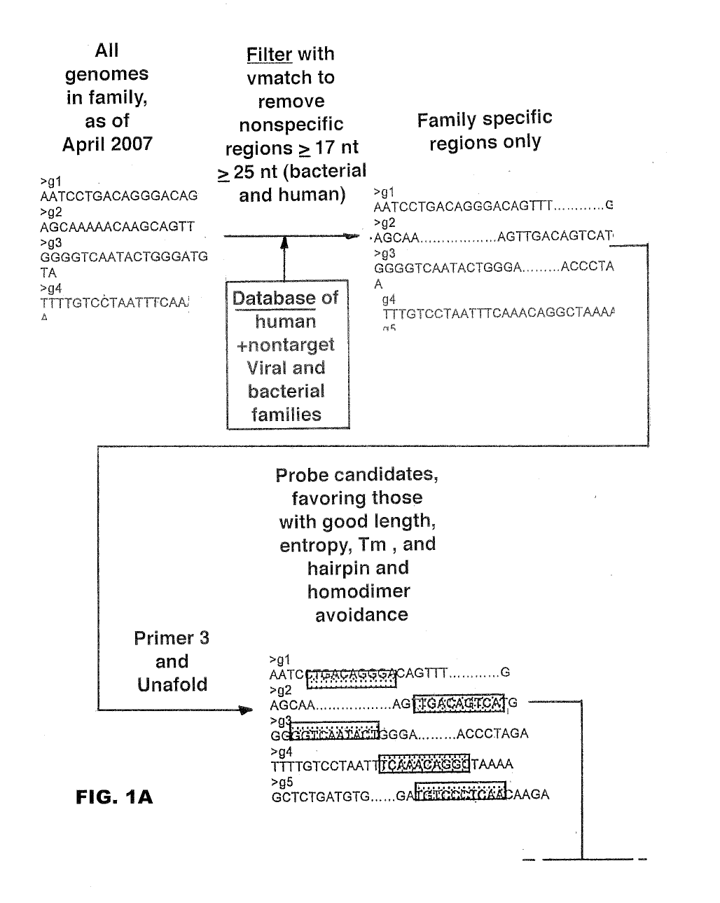

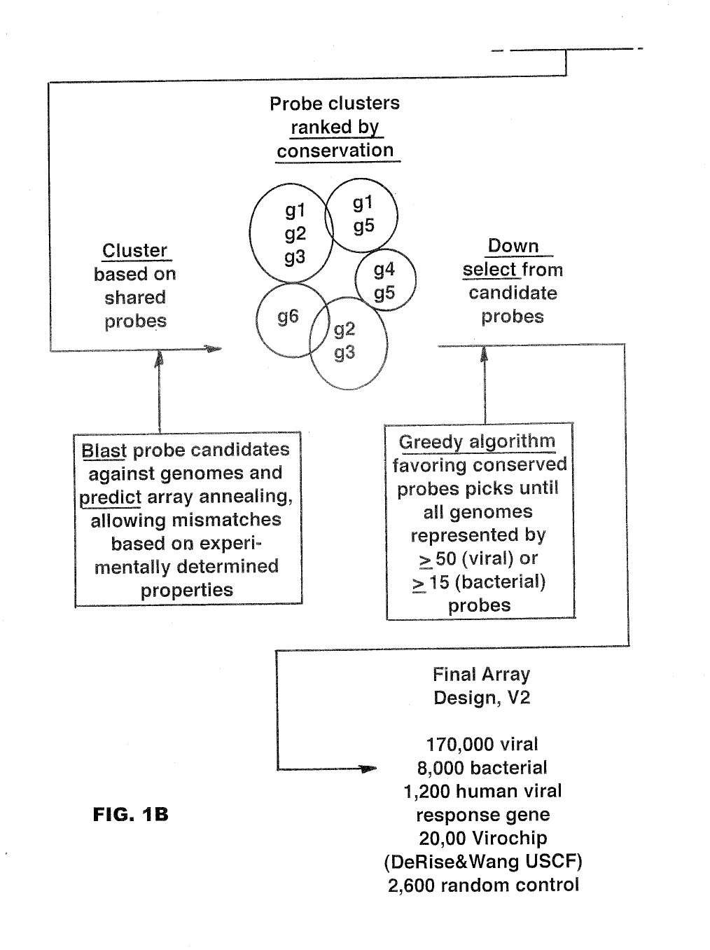

FIGS. 1A and 1B provide an illustration of a process used to obtain the oligonucleotide probe sequences in accordance with the present disclosure.

An initial genomic collection can be obtained, for example, by downloading a complete bacterial (e.g. eubacteria), fungal, archaea, protozoan, and viral genomes, segments, and plasmid sequences from public sources such as Baylor College of Medicine Human Genome Sequencing Center (BCM-HGSC), Broad Institute, Global Initiative on Sharing All Influenza Data (GISAID), Integrated Genomics, Microgen, University of Oklahoma, Poxvirus Bioinformatics Resource Center, Genome Institute of Singapore, Stanford Genome Technology Center (SGTC), The Institute for Genomic Research (TIGR), University of Minnesota, Washington University Genome Sequencing Center, NCBI Genbank, the Integrated Microbial Genomics (IMG) project at the Joint Genome Institute, the Comprehensive Microbial Resource (CMR) at the JC Venter Institute, RepBase, SILVA, and The Sanger Institute in the United Kingdom, as well as proprietary sequences from nonpublic sources. The sequence data is then organized by family for all organisms or targets. For the embodiment of Version 3 (v3) of the array of the present disclosure, all available partial sequences were included in the target sequence collection as well as complete genomes. For the embodiment Version 5 (v5) array, probes were screened for uniqueness relative to ribosomal RNA sequences of the SILVA database, repetitive sequence from the RepBase database, and human sequence data that includes all contigs assembled onto chromomes and contigs that have not been assembled onto chromosomes.

It has been shown that the length of longest perfect match (PM) is a strong predictor of hybridization intensity, and that for probes at least 50 nucleotide (nt) long, a PM≦20 base pairs (bp) have signal less than 20% of that with a PM over the entire length of the probe. Therefore, for each target family, regions with perfect matches to sequences outside the target family were eliminated. In particular, a match threshold was identified in accordance with the present disclosure. Using, e.g., the suffix array software vmatch (see reference 6), perfect match subsequences of, e.g., at least 17 nt long present in non-target viral families or, e.g., 25 nt long present in the human genome or non-target bacterial families were eliminated from consideration as possible probe subsequences or, e.g. 19 nt or 20 nt for all taxa. Sequence similarity of probes to non-target sequences below this threshold was allowed. As shown later in the present disclosure, such similarity can be accounted for using a statistical log likelihood algorithm, later described. According to an embodiment of the disclosure, from these family-specific regions, probes 50-66 bases long were designed for one family at a time or probes 40-60 bases long were designed for one family at a time. Candidate probes were generated using, for example, MIT's Primer3 software. See, e.g., Steve Rozen, Helen J. Skaletsky (1998) Primer3 with minor configuration modification to allow the design of probes up to 70 bp, up from the 36 bp program default.

According to several exemplary embodiments of the disclosure, the following Primer3 settings were modified from the default values:

PRIMER_TASK=pick_hyb_probe_only

### PRIMER_PICK_ANYWAY=1

### PRIMER_INTERNAL_OLIGO_OPT_SIZE=55

### PRIMER_INTERNAL_OLIGO_MIN_SIZE=50

### PRIMER_INTERNAL_OLIGO_MAX_SIZE=60 or 70

### PRIMER_INTERNAL_OLIGO_OPT_TM=90

### PRIMER_INTERNAL_OLIGO_MIN_TM=80

### PRIMER_INTERNAL_OLIGO_MAX_TM=110

### PRIMER_INTERNAL_OLIGO_MIN_GC=25

### PRIMER_INTERNAL_OLIGO_MAX_GC=75

### PRIMER_NUM_NS_ACCEPTED=0

### PRIMER_EXPLAIN_FLAG=0

### PRIMER_FILE_FLAG=1

### PRIMER_INTERNAL_OLIGO_SALT_CONC=450

### PRIMER_INTERNAL_OLIGO_DNA_CONC=100

### PRIMER_INTERNAL_OLIGO_MAX_POLY_X=4

These settings identify candidate probes in the desired length range, melting temperature (Tₘ) range, GC % range, and without homopolymer repeats longer than 4 (i.e. regions with AAAAA, GGGGG, etc. are not selected as probe candidates).

The above step was followed by Tₘ and homodimer, hairpin, and probe-target free energy (ΔG) prediction using, for example, Unafold (see, e.g., Markham, N. R. & Zuker, M. (2005) DINAMeIt web server for nucleic acid melting prediction. Nucleic Acids Res., 33, W577-W581). Homodimers occur when an oligo hybridizes to another copy of the same sequence, and hairpining occurs when an oligo folds so that one part of the oligo hybridizes with another part of the same oligo. According to an embodiment of the disclosure, candidate probes with unsuitable ΔG's, GC % or Tₘ's were excluded as described in reference 8. Desirable range for these parameters was 50≦length≦66, Tₘ≧80° C., 25%≦GC %≦75%, trimer entropy>4.5, ΔG_{homodimer}=ΔG of homodimer formation >15 kcal/mol, ΔGₕₐᵢᵣₚᵢₙ=ΔG of hairpin formation >−11 kcal/mol, and ΔG_{adjusted}=ΔG_{complement}−1.45 ΔGₕₐᵢᵣₚᵢₙ−0.33 ΔG_{homodimer}<−52 kcal/mol. In some cases, related for example to bacterial probes, an additional minimum sequence complexity constraint was enforced, requiring a trimer frequency entropy of at least 4.5.

More generally, in accordance with the above embodiments, probes with suitable annealing characteristics or preferred binding properties (e.g., polynucleotides from target specific regions with favored thermodynamic characteristics) were selected, in order to remove probes that are likely to bind to non-target sequences, whether the non-target sequence is the probe itself or a low complexity non-specific sequence. In some exemplary embodiments, candidate probes that can produce non-specific binding due to long stretches of G's, such as GGGGGGGG, in the candidate probe sequence are modified where another nucleotide, such as T, as an alternate candidate probe sequence, such as GGGGTGTG. If fewer than a user-specified minimum number of candidate probes per target sequence (the specific value of which can depend upon the particular application needs and available number of probes on a particular array platform) passed all the criteria, then those criteria were relaxed to allow a sufficient number of probes per target. For example, a skilled person can relax the number of mismatches in a sequence or the length of the probe. In accordance with a relaxation embodiment, candidates that passed the above mentioned first step but failed the above mentioned second step can be allowed. If no candidates passed the first step, then regions passing target-specificity (e.g. family specific) and minimum length constraints can be allowed.

From these candidates, probes were selected in decreasing order of the number of targets represented by that probe (i.e., probes detecting more targets in the family were chosen preferentially over those that detected fewer targets in the family), where a target was considered to be represented if, for example, a probe matched it with at least 85% sequence similarity over the total probe length, and a perfectly matching subsequence of at least 29 contiguous bases spanned the middle of the probe. It should be noted that the perfect-match stretch did not have to be centered, and in fact data gathered by the applicants indicate, in some embodiments, higher probe sensitivity if the match falls toward the 5′ end of the probe (for probes tethered to the solid support at the 3′ end), so long as it extends over the middle of the probe. In some embodiments, a target is considered represented if, for example, a probe matched it with at 85% sequence identity or similarity to the target over the length of the probe and is predicted to detect the target from an empirically driven predictor. An empirically driven predictor can be, for example, a linear predictor based on an alignment score (such as BLAST bit scores), the predicted Tm of the probe to its matching target sequence, and the start position of the match on the probe, also known as a “hit start”.

For probes that tie in the number of targets represented, a secondary ranking was used to favor probes most dispersed across the target from those probes which had already been selected to represent that target. The probe with the same conservation rank that occurs at the farthest distance from any probe already selected from the target sequence is the next probe to be chosen to represent that target. In some embodiments, candidate probes can be further refined or clustered based on the downstream applications of the probes. For example, to avoid providing many highly similar candidates from the same region of a genome, candidate probes can be clustered from a family that had been designed based on the uniqueness and thermodynamic methods, already described, by sequence similiarity. In one embodiment of this disclosure (v5), candidate probes were clustered so that probes with more than 90% sequence identity were in the same cluster allowing one a single representative of each cluster to be retained and removing the other near-identical candidate probes in that cluster.

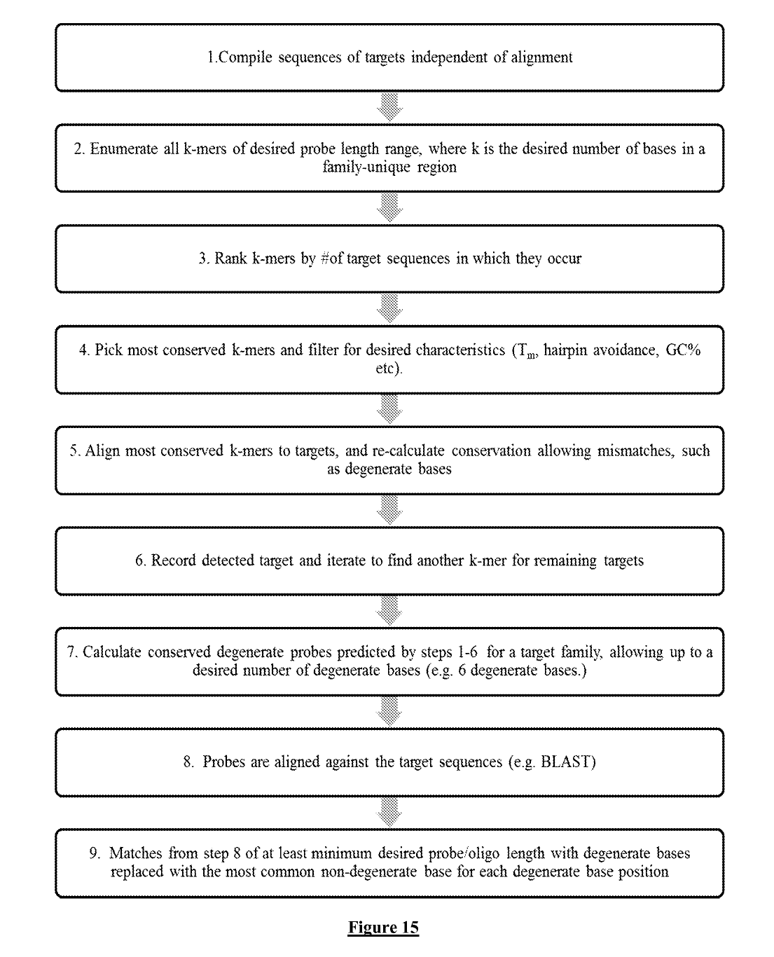

According to an exemplary embodiment of this disclosure (v5), candidate probes can be a k-mer probe, generated by using k-mer statistics (see reference 33). The term “k-mer” as described herein refers to a specific n-tuple of nucleic acid sequences, such as DNA. Generation of candidate probes using k-mer statistics can be performed by the following (see FIG. 15): 1) compiling sequences of targets independent of any alignment; 2) enumerating all k-mers of a desired probe length range, where k is the desired number of bases of a probe in a family-unique region; 3) ranking k-mers by the number of target sequences in which they occur, 4) picking conserved k-mers and filtering for desired characteristics (Tₘ, hairpin avoidance, GC % etc); 5) aligning conserved k-mers to targets, and re-calculate conservation allowing mismatches, such as degenerate bases; 6) recording detected target and iterate to find another k-mer for remaining targets; 7) calculating conserved degenerate probes predicted by steps 1-6 for a target family, allowing up to a desired number of degenerate bases (e.g. 6 degenerate bases.); 8) aligning probes against target sequences (e.g. BLAST); and 9) selecting probes from the matches of step 8 that satistfy at least a minimum desired probe/oligo length and replacing degenerate bases with the most common non-degenerate base for each degenerate base position. Candidate probes from k-mer statistics, or k-mer probes or Primux k-mer probes, can be used in addition or in alternative to the methods to generate candidate probes based on PM described above. A candidate probe from one method can have the same sequence from another method. A person with ordinary skill can choose to eliminate repeats of the same candidate probe when generated probes for an array. Parameters, or desired characteristics, for candidates probes generated by k-mers in one exemplary embodiment of this disclosure (v5) include the following: A length 50-60 bp, a maximum homopolymer length 5, a targeted minimum 40 probes per target sequence, a minimum trimer entropy of 4.5, a minimum hairpin energy of G=−11 kcal/mol, minimum dimer energy of G=−15 kcal/mol, a Tₘ between 85° C. and 130° C., and a GC % in the range 20-80%. A person of ordinary skill can adjust or relax these exemplary parameters or other desired parameters based the downstream application of the candidate probes. For example, a person of ordinary skill can relax the targeted minimum number of probes per target sequence when there were insufficient probe candidates passing the specifications above. In an embodiment of the present disclosure (v5), k-mer probes, after filtering for desired characteristics, were BLASTed against target sequences and matches of at least 40 bases in length were identified as candidate probes. A consensus sequence was determined for candidate probes with up to 6 degenerate bases, where the most common non-degenerate base was replaced for each degenerate base position.

In several embodiments, arrays contained probes representing all complete viral genomes or segments associated with a known viral family, with at least 15 probes per target (Table 1). For example, a first exemplary array obtained by applicants (array v1) did not include unclassified targets not designated under a family. On a second example of array obtained by applicants (v2 array), every viral genome or segment was represented by at least 50 probes, totaling 170,399 probes, except for 1,084 viral genomes that were not associated under a family-ranked taxonomic node (“nonConforming sequences”). These had a minimum of 40 probes per sequence totaling 12,342 probes. There were a minimum of 15 probes per bacterial genome or plasmid sequence, totaling 7,864 probes on the v2 array. Bacterial genomes that were not associated under a family-ranked taxonomic node were not included in the v2 array design. In another example obtained by applications (array v5), every target sequence was represented by at least 30 probes selected from conservation-favoring probes and at least 5 probes selected from discriminating probes.

TABLE 1
Summary of v1 and v2 array design - Probe Counts

| Number of Probes | Probe Description |
|---|---|
| Version 1 | |
| 36497 | Viral detection probes (15 probes/target from each |
| | taxonomic family) |
| 20736 | Wang, deRisi Virochip probes |
| 1278 | human viral response genes |
| 3000 | random controls |
| Version 2 | |
| 170399 | Viral probes (50 probes/target from each taxonomic |
| | family) x 2 replicates |
| 12342 | nonConforming viruses (not associated w/taxonomic |
| | family, 40 probes/target) |
| 7864 | bacterial probes (15probes/target) |
| 20736 | Wang, deRisi Virochip probes |
| 1278 | human viral response genes |
| 2651 | random controls |

On both arrays v1 and v2, as controls for the presence of human DNA/mRNA from clinical samples, 1,278 probes to human immune response genes were designed. For targets, the genes for GO:0009615 (“response to virus”) were downloaded from the Gene Ontology AmiGO website (http://amigo.geneontology.org), filtering for Homo sapiens sequences. There were 58 protein sequences available at the time (Jul. 12, 2007), and from these, the gene sequences of length up to 4× the protein length were downloaded from the NCBI nucleotide database based on the EMBL ID number, resulting in 187 gene sequences. Fifteen probes per sequence were designed for these using the same specifications as for the bacterial and viral target probes.

To assess background hybridization intensity, ˜2,600 random control probe sequences were designed that were length and GC % matched to the target probes on arrays such as v1, v2, v3, or v5. These had no appreciable homology to known sequences based on BLAST similarity.

In addition, 21,888 probes from the Virochip version 3 from University of California San Francisco (see references 3, 21, 22, 23) were included on array v1 and v2.

In several embodiments including further exemplary arrays obtained by applicants (arrays v3.1, v3.2, v3.3, and v3.4), sequence data was downloaded as summarized in Table 2 for all viral, bacterial, and fungal sequences, and species of protozoa that infect humans and near neighbors of those protozoa species. All sequences from the LLNL KPATH, JCVI, IMG, and NCBI Genbank databases were included, whether it represented complete genomes, partial sequences, genes, noncoding fragments, etc.

In order to reduce the number of redundant viral sequences, cd-hit (see reference 26) was used to cluster the sequences within each group or family of viral sequences into clusters sharing 98% identity, and using only the longest sequence representative from each cluster for conserved probe design. This reduced the number of nonredundant viral targets by ˜70% compared to the full set with numerous duplicate and near-duplicate sequences. In order to reduce probe redundancy and biased coverage for species with large numbers of sequences for highly similar strain variants, duplicate and highly similar probes (e.g. ≧90%) from a complied list of conserved probes, discriminating probes, and k-mer probes were clustered and the total probe set was reduced by taking only the longest probe representing each cluster in an exemplary embodiment of this disclosure (v5). A skilled person can also reduce the number of probes based on the number of synthesis cycles required by a probe on a desired array. For example, Version 5 truncated probes requiring more than 148 synthesis cycles on the NimbleGen platform.

As in other embodiments, the vmatch software (see reference 6) can be used as described above, to eliminate non-unique regions of a target group (e.g. a viral or bacterial family) relative to other families and kingdoms, or species for the case of protozoa. Bacterial and viral probes were designed to be unique relative to one another and the human genome, but were not checked for uniqueness against fungal and protozoa sequences. In an exemplary embodiment of this disclosure, array v5, protozoa were not screened to eliminate non-unique regions relative to other families of protozoa but were screened relative to the other kingdoms, RepBase and SILVA databases, and the human genome. In one exemplary embodiment, protozoa probes can be screened to eliminate non-unique regions relative to other families of protozoa to obtain more specific probes for each genus and species. Uniqueness against sequences in the same kingdom was not required for groups without family classification. Fungal and protozoa sequences were checked against one another as well as against human, viral, and bacterial genomes for uniqueness. From the unique regions, a candidate pool of probes was designed that passed Tₘ, length, GC %, entropy, hairpin, and homodimer filters as for previously described embodiments, relaxing these constraints where necessary to obtain sufficient numbers of probes per target.

Some sequences did not contain enough unique subsequences from which to design probes, for example, many rRNA sequences are conserved across different families or even kingdoms so are not appropriate for family identification, and probes for these were not designed. Probes conserved within a family or within subclades of a family (e.g. genus, species, etc.), yet still unique relative to other families and kingdoms, were selected as described above for array v2, favoring probes conserved within a family or other grouping (e.g. a virus group without family classification or a protozoa species). That is, Applicants selected probes in decreasing order (i.e. probes detecting more targets in the family were chosen preferentially over those that detected fewer targets in the family) of the number of targets represented by that probe, where a target was considered to be represented if a probe matched it with at least 85% sequence similarity over the total probe length, and a perfectly matching subsequence of at least 29 contiguous bases spanned the middle of the probe. In another embodiment, Applicants selected probes in decreasing order (i.e. probes detecting more targets in the family were chosen preferentially over those that detected fewer targets in the family) of the number of targets represented by that probe, where a target was considered to be represented if a probe matched it 85% homology to the target over the length of the probe and is predicted to detect the target from an empirically driven predictor.

It should be noted that probes are unique relative to other non-target families and kingdoms, but are conserved to the extent possible within the target group (e.g. family grouping or in the case of protozoa, species group). The conserved, or “discovery” probes are aimed to detect novel unsequenced organisms that may be likely to share the same conserved regions as have been observed in previously sequenced organisms.

In some embodiments, in eliminating non-unique regions of a target group (e.g. a viral or bacterial family) relative to other target groups or subgroups (e.g. families and kingdoms, or species for target groups such as protozoa) can be performed using for example a suitable software such as vmatch software (see reference 6). For example a software such as vmatch can be used to provide bacterial and viral probes designed to be unique relative to one another and the human genome. In some embodiments, eliminating non-unique regions can comprise checking the sequence against additional groups and/or subgroups of target in accordance with a desired experimental design. In particular, the bacterial and viral probes designed to be unique relative to one another and the human genome can also be checked for uniqueness against additional fungal, bacterial, and archaeal sequences. The number and selection of target groups that can be used to perform eliminating non-unique sequence can vary and be selected in accordance with a desired specificity as will be understood by a skilled person.

For example, in some embodiments, in addition to eliminating non-unique regions of a target group (e.g. a viral or bacterial family) relative to other families and kingdoms, or species for the case of protozoa using vmatch software (see reference 6) to provide bacterial and viral probes designed to be unique relative to one another and the human genome, the groups were also checked for uniqueness against ribosomal sequences outside of the target domain. For example, probes for bacterial families could have matches to bacterial ribosomal RNA but not to ribosomal RNA sequences from human, fungal, etc.

In further exemplary embodiments, in addition to eliminating non-unique regions of a target group (e.g. a viral or bacterial family) relative to other families and kingdoms, or species for the case of protozoa using vmatch software (see reference 6) to provide bacterial and viral probes designed to be unique relative to one another and the human genome, the groups were also checked for uniqueness to ribosomal sequences and fungal bacterial, and archaeal sequences as seen in Example 11.

According to further embodiments of the present disclosure, probes can be chosen by other alternative criteria, for example, by selecting probes chosen from dispersed positions in each target sequence to represent regions in different parts of each genome, which could be useful, for example, in detecting chimeric sequences. Another criteria could be to select probes chosen to be shared across as many sequences as possible, regardless of family specificity, so that probes shared across multiple families and even kingdoms would be preferred. The above criteria are based on the fact that evolutionarily-related organisms contain sufficient nucleotide sequence conservation, in at least some genomic region(s), to be exploited at the desired taxonomic resolution level.

Several array designs of conserved probes were created with different probe densities, differing in the number of probes per target sequence, as indicated in the Table 2 and Table 2.1. Total probe counts (Table 3 and Table 3.1) indicate those remaining after removing duplicate probes. The design platform in Table 3 includes the company and the number of probes (probe density) on the array, although the list of platforms and companies is not an exclusive list because a skilled person can adapt the array with the probes based on the platform of choice. These are the platforms that that the applicants have worked with experimentally. The NimbleGen® 3×720K array by Roche can test 3 samples at a time with 720,000 probes, as it is essentially the 2.1 M probe density array divided into 3 areas. Other platforms known to a skilled person include arrays produced from Agilent® and Illumina®.

TABLE 2
Array versions 3.1, 3.2, 3.3., and 3.4 - Probe count breakdown

| Number | | |
|---|---|---|
| of | | |
| Probes | Target Type | Probes per sequence (pps) Minimum design goal |
| MDA | | |
| v3.1 | | |
| 893961 | Bacteria Family | 30 pps |
| 263586 | Bacteria Family | 30 pps |
| | Unclassified | |
| 346957 | Viral Family probes | 30 pps |
| 16686 | Viral Family Unclassified | 30 pps |
| 1875 | SFBB (novel sequences | Tiled adjacent, no overlap between probes |
| | from UCSF Blood Systems | |
| | Research Institute) | |
| 157050 | Fungal probes | 5 pps |
| 137939 | Protozoa probes | 5 pps |
| 1833 | Additional Hemorrhagic | |
| | fever virus probes, same as | |
| | MDA v2 | |
| 3438 | random controls (Len and | |
| | GC distribution matching | |
| | census and design3 MDA | |
| | probes) | |
| 1802110 | Total | MDA High Density Probes |
| MDA | | |
| v3.2 | | |
| and | | |
| v3.3 | | |
| 222574 | Bacteria Family | 10 pps for complete genomes and plasmids in every |
| | | family; plus 10 pps for genes and fragments in 248 |
| | | smaller families; plus 1 pps for genes and sequence |
| | | fragments in the 32 families with the most sequence |
| | | data |
| 49016 | Bacteria Family | 5 pps |
| | Unclassified | |
| 137855 | Viral Family probes | 10 pps for all sequences, both complete and |
| | | fragments |
| 5747 | Viral Family Unclassified | 10 pps for all sequences, both complete and |
| | | fragments |
| 1875 | SFBB | Tiled across each sequence with 0 overlap, i.e. each |
| | | base has probe coverage of 1. Unpublished sequence |
| | | targets of novel viruses provided by Eric Delwart's |
| | | group at the Blood Systems Research Institute, |
| | | University of California, San Francisco, CA (abbrev |
| | | SFBB = SF Blood Bank) |
| 157050 | Fungal probes | 5 pps |
| 137939 | Protozoa probes | 5 pps |
| 1833 | Additional Hemorrhagic | |
| | fever virus probes, same as | |
| | MDA v2 | |
| 3469 | random controls (Len and | |
| | GC distribution matching | |
| | census and design1 MDA | |
| | probes) | |
| 713743 | Total | MDA Medium Density Probes |
| v3.4 | | |
| 161451 | Bacteria Family | 10 pps for complete genomes and plasmids in every |
| | | family; plus 10 pps for genes and fragments in 248 |
| | | smaller families; |
| 49016 | Bacteria Family | 5 pps |
| | Unclassified | |
| 137855 | Viral Family probes | 10 pps for all sequences, both complete and fragments |
| 5747 | Viral Family Unclassified | 10 pps for all sequences, both complete and fragments |
| 1875 | SFBB | Tiled across each sequence with 0 overlap, i.e. each |
| | | base has probe coverage of 1 |
| 1833 | Additional Hemorrhagic | |
| | fever virus probes, same as | |
| | MDA v2 | |
| 2562 | random controls | |
| 357532 | Total | MDA Low Density Probes |

TABLE 2.1
Array version 5 (v5) - Probe count breakdown

| | | |
|---|---|---|
| Number of | Target | |
| Probes | Type | Minimum design goal |

360K format

| | | |
|---|---|---|
| 194207 | Viral | 30 from conserved algorithm |
| 126172 | Bacterial | 5 from discriminating algorithm (discriminating |
| 7860 | Archaeal | may be the same as conserved, so after removing |
| 10690 | Protozoa | duplicates there may be only 30 total) |
| 18793 | Fungi | |

135K format

| | | |
|---|---|---|
| 84586 | Viral | 15 from conserved algorithm |
| 35944 | Bacterial | 2 from discriminating algorithm (discriminating |
| 2811 | Archaeal | may be the same as conserved, so after removing |
| 3829 | Protozoa | duplicates there may be only 15 total) |
| 3951 | Fungi | |

TABLE 3
Array versions 3.1, 3.2, 3.3, and 3.4 - Total probe counts

| | | Array Platform (# | | |
|---|---|---|---|---|
| Probe | | indicates Probe | | MDA |
| Counts | | density) | Probes included | Version |
| 2062997 | Total | Nimblegen 2.1M | MDA High Density | 3.1 |
| | | | Probes + Census probes | |
| 937649 | Total | Agilent 1M | MDA Medium Density | 3.2 |
| | | | Probes + Census probes | |
| 713743 | Total | NimbleGen3 × | MDA Medium Density | 3.3 |
| | | 720K | Probes | |
| 357532 | Total | Nimblegen 388K | MDA Low Density | 3.4 |
| | | | Probes | |

TABLE 3.1
Array version 5 (v5) - Total probe counts

| | | Array Platform | | |
|---|---|---|---|---|
| | | (# | | |
| Probe | | indicates Probe | | MDA |
| Counts | | density) | Probes included | Version |
| 134896 | Total | Nimblegen | Subset of MDAv5 from | V5 |
| | | 12 × 135K Or | families in which there | Clinical |
| | | Agilent 4 × | are species known to | chip |
| | | 180K | infect vertebrates; random | |
| | | | negative controls; and | |
| | | | Thermotoga positive | |
| | | | controls | |
| 361863 | Total | Nimblegen 3 × | Probes for all families and | V5 |
| | | 720K Or | family unclassified | 360K |
| | | Nimblegen 1 × | sequences; random | |
| | | 388K Or | negative controls; and | |
| | | Agilent 2 × | Thermotoga positive | |
| | | 400K | controls | |

Probe counts represent numbers after removing duplicate probes, which may occur between census and discovery probes or between family unclassified and family classified viruses (or bacteria).

“Conserved” probes are probes conserved across multiple sequences from within a family or other (e.g. protozoa species, or family-unclassified viral group) target set, but not conserved across families or kingdoms. Such probes aim to detect known organisms or discovery novel organisms that have not been sequenced which possess some sequence homology to organisms that have been sequenced, particularly in those regions found to be conserved among previously sequenced members of that family or other target group. These conserved probes may identify an organism to the level of genus or species, for example, but may lack the specificity to pin the identification down to strain or isolate.

In several embodiments, an alternative method of selecting probes was used in order to select the least conserved, that is, the most strain or sequence specific probes. These probes were termed “census probes” or “discriminating probes”. Such census/discriminating probes, aim to fill the goal of providing higher level discrimination/identification of known species and strains, but may fail to detect novel organisms with limited homology to sequenced organisms. Census probes were designed to provide greater discrimination among targets to facilitate forensic resolution to the strain or isolate level. As in the foregoing description and similar to other embodiments, a greedy algorithm was employed, however in this case the probes matching the fewest target sequences were favored. Probes were selected from the pool of probe candidates passing the Tₘ, length, GC %, entropy, hairpin, and homodimer filters when possible.

As also mentioned above, these constraints were relaxed if necessary to obtain sufficient probes per sequence for targets with adequate unique regions. For every target sequence, probes were selected in ascending order of the number of targets represented by that probe, where a target was considered to be represented if a probe matched it with, for example, at least 85% sequence similarity over the total probe length, and, for example, a perfectly matching subsequence of at least 29 contiguous bases spanned the middle of the probe or if a probe matched it with, for example, at 85% homology to the target over the length of the probe and is predicted to detect the target from an empirically driven predictor. By ascending order, it is meant that probes were sorted in increasing order of the number of targets each represents, and for each target sequence probes were picked from the list in order of those that detected the fewest other target sequences. According to some embodiments, probes were continually selected for a target until at least suitable 10 probes per sequence were identified. According to some embodiments, probes were continually selected until at at least more than 10 probes were identified, such as 15, 30, or 40 probes per target sequence. According to some embodiments, probes were continually selected for a target for a ratio of conservation favoring probes to discriminating probes, for example 30 conservation favoring probes to 5 discriminating probes per target sequence. Due to the large number of Orthomyxoviridae sequences, only 5 probes per sequence were included for this family in some embodiments. In this way, the most sequence-specific probes were selected, accumulating probes in order of sequence-specificity until the desired number of probes per target was obtained.

Census probes were designed for all the viral and bacterial complete genomes, segments, and plasmids, as indicated in Table 4. Discriminating probes used in one embodiment of this disclosure (v5) was designed for all viral, bacterial, fungal, archaeal, and protozoan complete genomes, chromosomes, segments, and plasmids are included in the counts indiated in Table 2.1. Viral sequences were not clustered using cd-hit as in the foregoing description of conserved probes, since it was desired that the census probes discriminate every isolate, if possible, even if those isolates had more than 98% identity. For v3, census probes were also designed for sequence fragments for those bacterial families with less available sequence data, although not for the 32 families with the most available sequence data since they were already so well-represented by the probes for the large amount of complete sequences available and the additional probes representing the fragmentary and partial sequences was thought to be unnecessary for the goal of censusing for strain discrimination.

TABLE 4
Census Probe Counts

| | | |
|---|---|---|
| 307086 | Bacteria Family | 10 pps, whole genomes for all |
| | | families, fragments for 248 smaller |
| | | families, but not fragments for 32 |
| | | families with the most sequence |
| | | data |
| 1691 | Bacteria Family | 10 pps |
| | Unclassified | |
| 84597 | Viral Family probes except | 10 pps |
| | Orthomyxoviridae | |
| 9934 | Viral Family Unclassified | 10 pps |
| 15118 | Orthomyxoviridae | 5 pps |
| 418363 | Total | |

In several embodiments, a multiplex array was designed using the oligonucleotide probes designed according to the method herein disclosed. In particular, the NimbleGen platform supports a 4-plex configuration. This uses a gasket to divide a slide into 4 individual subarrays, enabling the testing of 4 samples at a time on a single slide and lowering the cost per sample. Up to 72,000 probe sequences can be tiled within each subarray.

To take advantage of this configuration, a modified version v2 of the array according to the present disclosure was built with 70,916 unique probe sequences. Array v2 as described above has 215,270 probe sequences, representing each virus genome or segment by at least 50 probes. In a smaller v2.1 array, each virus genome or segment is represented by 10-20 probes, as indicated in Table 5. The same process was used to downselect from the candidate pool of probes as was described in paragraph 0055, as before favoring probes that were more conserved within the target group and breaking ties by picking the most distant probe in a target genome from other probes that were already selected for that target, building up the total until all viral genomes and segments were represented by the user-specified (10 or 20) number of probes. The same bacterial probes were used as on the array v2, and the probes from the Virochip and human viral response genes were omitted.

TABLE 5
Reduced probe set multiplex array v2.1

| Number of | Probes per | |
|---|---|---|
| probes | sequence | Target Sequences |
| 48893 | 20 | All Viral families except Orthomyxoviridae and |
| | | family unclassified complete viral genomes |
| | | and segments |
| 7777 | 10 | Segments in the Orthopox family |
| 2972 | 10 | Family unclassified viral genomes and complete |
| | | segments |
| 7864 | 15 | Bacterial genomes and plasmids |
| 3410 | — | Random controls with GC % and length |
| | | distribution matched to target probes |
| 70916 | | Total |

In some embodiments, an oligonucleotide probe for detection of targets in a target group is described, the oligonucleotide probe being in combination with at least four other oligonucleotide probes, wherein: the oligonucleotide probe has a sequence selected from the group consisting of SEQ ID NO 1-133,263; and the target group comprises a group of microorganisms such as the microorganisms exemplified in Example 10. In some embodiments, an oligonucleotide probe for detection of targets in a target group is described, the oligonucleotide probe being in combination with at least four other oligonucleotide probes, wherein: the oligonucleotide probe has a sequence selected from the group consisting of SEQ ID NO 133,264-534,156; and the target group comprises a group of microorganisms such as the microorganisms exemplified in Example 16

In some embodiments the oligonucleotide probe has a sequence selected from the group consisting of SEQ ID NO's 1-63 and 446-5,722; and the group of microorganisms comprises a bacterial group such as the bacterial group exemplified in Example 10. In some embodiments the oligonucleotide probe has a sequence selected from the group consisting of SEQ ID NO's 141, 124-267, 772 and 491,511-492,337 and 496,379-512,129 and 615,629-650,745; and the group of microorganisms comprises a bacterial group such as the bacterial group exemplified in Example 16.

In some embodiments the oligonucleotide probe has a sequence selected from the group consisting of SEQ ID NO's 64-445; 5,723-133,263; 362-445; 17545-17929; and 48,275-91,627; and the group of microorganisms comprises a viral group such as the viral group exemplified in Examples 10 and 11. In some embodiments the oligonucleotide probe has a sequence selected from the group consisting of SEQ ID NO's 297,256-491,462 and 492,545-495,658 and 515,887-534,156 and 534,157-615,628; and the group of microorganisms comprises a viral group such as the viral group exemplified in Example 16.

In some embodiments the oligonucleotide probe has a sequence selected from the group consisting of SEQ ID NO's 362-445, 17,545-17,929 and 48,275-91,627; and the group of microorganisms comprises a flu group such as the flu group exemplified in Examples 10 and 11.

In some embodiments the oligonucleotide probe has a sequence selected from the group consisting of SEQ ID NO's 286,566-297,255 and 492,437-492,544 and 514, 810-515,886 and 657,361-661,081; and the group of microorganisms comprises a group of species of protozoa such as exemplified in Example 16.

In some embodiments the oligonucleotide probe has a sequence selected from the group consisting of SEQ ID NO's 133,264-141,123 and 491,463-491,510 and 495,659-496,378 and 650,746-653,508; and the group of microorganisms comprises an archaeal group such as exemplified in Example 16.

In some embodiments the oligonucleotide probe has a sequence selected from the group consisting of SEQ ID NO's 267, 773-286, 565 and 492,338-492, 436 and 512,130-514,809 and 653,509-657,360; and the group of microorganisms comprises fungal group such as exemplified in Example 16.

In some embodiments the oligonucleotide probe is capable of detecting at least one species selected from table 10 such as the species exemplified in Example 10 as seen in Examples 10 and 11.

In some embodiments the oligonucleotide probe is capable of detecting at least one species from a family of species selected from the following families, or closest taxonomically labeled group to family for sequences unclassified at the family level:

### Bacteria:

Acaryochloris, Acetobacteraceae, Acholeplasmataceae, Acidaminococcaceae, Acidimicrobiaceae, Acidithiobacillaceae, Acidobacteriaceae, Acidothermaceae, Actinomycetaceae, Actinosynnemataceae, Aerococcaceae, Aeromonadaceae, Alcaligenaceae, Alcanivoracaceae, Alicyclobacillaceae, Alteromonadaceae, Alteromonadales, Anaerolinaceae, Anaplasmataceae, Aquificaceae, Arthrospira, Aurantimonadaceae, BD1-7_clade, Bacillaceae, Bacteriovoracaceae, Bacteroidaceae, Bacteroidales, Bartonellaceae, Bdellovibrionaceae, Beijerinckiaceae, Beutenbergiaceae, Bhargavaea, Bifidobacteriaceae, Blattabacteriaceae, Blautia, Brachyspiraceae, Bradyrhizobiaceae, Brevibacteriaceae, Brucellaceae, Burkholderiaceae, Burkholderiales, Caldilineaceae, Caldisericaceae, Caldithrix, Campylobacteraceae, Campylobacterales, Candidatus_Accumulibacter, Candidatus_Amoebophilus, Candidatus_Azobacteroides, Candidatus_Baumannia, Candidatus_Cardinium, Candidatus_Carsonella, Candidatus_Chloracidobacterium, Candidatus_Cloacamonas, Candidatus_Hodgkinia, Candidatus_Koribacter, Candidatus_Midichloria, Candidatus_Odyssella, Candidatus_Pelagibacter, Candidatus_Puniceispirillum, Candidatus_Sulcia, Candidatus_Tremblaya, Cardiobacteriaceae, Carnobacteriaceae, Catenulisporaceae, Caulobacteraceae, Cellulomonadaceae, Chitinophaga, Chlamydiaceae, Chlorobiaceae, Chloroflexaceae, Chromatiaceae, Chroococcales, Chrysiogenaceae, Chthoniobacter, Clostridiaceae, Clostridiales, Clostridiales_Family_XI, Clostridiales_Family_XIII, Clostridiales_Family_XVII, Clostridiales_Family_XVIII, Colwelliaceae, Comamonadaceae, Conexibacteraceae, Congregibacter, Coriobacteriaceae, Corynebacteriaceae, Coxiellaceae, Crocosphaera, Cryomorphaceae, Cyanobium, Cyanothece, Cyclobacteriaceae, Cystobacteraceae, Cytophagaceae, Deferribacteraceae, Dehalococcoides, Dehalogenimonas, Deinococcaceae, Dermabacteraceae, Dermacoccaceae, Dermatophilaceae, Desulfarculaceae, Desulfobacteraceae, Desulfobulbaceae, Desulfohalobiaceae, Desulfomicrobiaceae, Desulfovibrionaceae, Desulfurellaceae, Desulfurobacteriaceae, Desulfuromonadaceae, Dictyoglomaceae, Dietziaceae, Ectothiorhodospiraceae, Elusimicrobiaceae, Endoriftia, Enterobacteriaceae, Enterococcaceae, Entomoplasmataceae, Epulopiscium, Erysipelotrichaceae, Erythrobacteraceae, Eubacteriaceae, Exiguobacterium, Fangia, Ferrimonadaceae, Fibrobacteraceae, Fischerella, Flammeovirgaceae, Flavobacteriaceae, Flavobacteriales, Francisellaceae, Frankiaceae, Fusobacteriaceae, Gallionellaceae, Gemella, Gemmatimonadaceae, Geobacteraceae, Geodermatophilaceae, Gloeobacter, Glycomycetaceae, Gordoniaceae, Hahellaceae, Halanaerobiaceae, Halobacteroidaceae, Halomonadaceae, Haloplasmataceae, Halothiobacillaceae, Helicobacteraceae, Heliobacteriaceae, Herpetosiphonaceae, Holophagaceae, Hydrogenophilaceae, Hydrogenothermaceae, Hyphomicrobiaceae, Hyphomonadaceae, Idiomarinaceae, Ignavibacteriaceae, Intrasporangiaceae, Jonesiaceae, Kineosporiaceae, Kofleriaceae, Ktedobacteraceae, Lachnospiraceae, Lactobacillaceae, Legionellaceae, Lentisphaeraceae, Leptolyngbya, Leptospiraceae, Leptothrix, Leuconostocaceae, Listeriaceae, Lyngbya, Magnetococcus, Marinilabiaceae, Mariprofundaceae, Methylacidiphilaceae, Methylibium, Methylobacteriaceae, Methylococcaceae, Methylocystaceae, Methylophilaceae, Methylophilales, Micavibrio, Microbacteriaceae, Micrococcaceae, Microcoleus, Microcystis, Micromonosporaceae, Mitsuaria, Moraxellaceae, Moritellaceae, Mycobacteriaceae, Mycoplasmataceae, Myxococcaceae, Nakamurellaceae, Nannocystaceae, Natranaerobiaceae, Nautiliaceae, Neisseriaceae, Niabella, Niastella, Nitratifractor, Nitratiruptor, Nitrosomonadaceae, Nitrospiraceae, Nocardiaceae, Nocardioidaceae, Nocardiopsaceae, Nodosilinea, Nostocaceae, OM60_clade, Oceanospirillaceae, Opitutaceae, Oscillatoria, Oscillochloridaceae, Oscillospiraceae, Oxalobacteraceae, Paenibacillaceae, Parachlamydiaceae, Parvularculaceae, Pasteurellaceae, Pasteuriaceae, Patulibacteraceae, Pelobacteraceae, Peptococcaceae, Peptostreptococcaceae, Phycisphaeraceae, Phyllobacteriaceae, Piscirickettsiaceae, Planctomycetaceae, Planococcaceae, Polyangiaceae, Polymorphum, Porphyromonadaceae, Prevotellaceae, Prochlorococcaceae, Promicromonosporaceae, Propionibacteriaceae, Pseudo alteromonadaceae, Pseudoflavonifractor, Pseudomonadaceae, Pseudonocardiaceae, Psychromonadaceae, Puniceicoccaceae, Reinekea, Rhizobiaceae, Rhodobacteraceae, Rhodobacterales, Rhodocyclaceae, Rhodospirillaceae, Rhodospirillales, Rhodothermaceae, Rickettsiaceae, Rickettsiales, Rikenellaceae, Rubrivivax, Rubrobacteraceae, Ruminococcaceae, SAR11_cluster, SAR324_cluster, SAR86_cluster, SAR92_clade, Salinisphaeraceae, Sanguibacteraceae, Saprospiraceae, Segniliparaceae, Shewanellaceae, Simidua, Simkaniaceae, Sinobacteraceae, Solibacteraceae, Sphaerobacteraceae, Sphingobacteriaceae, Sphingomonadaceae, Spirochaetaceae, Spiroplasmataceae, Sporolactobacillaceae, Staphylococcaceae, Streptococcaceae, Streptomycetaceae, Streptosporangiaceae, Succinivibrionaceae, Sulfurovum, Sutterellaceae, Synechococcus, Synechocystis, Synergistaceae, Syntrophaceae, Syntrophobacteraceae, Syntrophomonadaceae, Teredinibacter, Thermaceae, Thermoactinomycetaceae, Thermoanaerobacteraceae, Thermoanaerobacterales_Family_III, Thermoanaerobacterales_Family_IV, Thermobaculum, Thermodesulfobacteriaceae, Thermodesulfobiaceae, Thermomicrobiaceae, Thermomonosporaceae, Thermos ynechococcus, Thermotogaceae, Thermotogales, Thiomonas, Thiotrichaceae, Thiotrichales, Trichodesmium, Tropheryma, Trueperaceae, Tsukamurellaceae, Turicella, Veillonellaceae, Verrucomicrobia_subdivision_{—}3, Verrucomicrobiaceae, Verrucomicrobiales, Vibrionaceae, Vibrionales, Victivallaceae, Waddliaceae, Xanthobacteraceae, Xanthomonadaceae, candidate_division_TM7, environmental_samples, sulfur-oxidizing_symbionts, unclassified_Actinobacteria, unclassified_Alphaproteobacteria, unclassified_Bacteria, unclassified_Bacteroidetes, unclassified_Betaproteobacteria, unclassified_Deltaproteobacteria, unclassified_Flavobacteriia, unclassified_Gammaproteobacteria, unclassified_SAR116_cluster, unclassified_Synergistetes, unclassified_Verrucomicrobia, unclassified_pseudomonads

### Viruses:

Adenoviridae, Alloherpesviridae, Alphaflexiviridae, Alvernaviridae, Ampullaviridae, Anelloviridae, Arenaviridae, Arteriviridae, Ascoviridae, Asfarviridae, Astroviridae, Bacillariodnavirus, Bacillariornaviridae, Bacillariornavirus, Baculoviridae, Barnaviridae, Begomovirus-associated_DNA_beta-like, Begomovirus-associated_alphasatellites, Benyvirus, Betaflexiviridae, Bicaudaviridae, Birnaviridae, Bornaviridae, Bromoviridae, Bunyaviridae, Caliciviridae, Caudovirales, Caulimoviridae, Chrysoviridae, Cilevirus, Circoviridae, Closteroviridae, Coronaviridae, Corticoviridae, Cystoviridae, Deltavirus, Dicistroviridae, Emaravirus, Endornaviridae, Filoviridae, Flaviviridae, Fuselloviridae, Gammaflexiviridae, Geminiviridae, Globuloviridae, Haloviruses, Hepadnaviridae, Hepeviridae, Herpesvirales, Herpesviridae, Hypoviridae, Idaeovirus, Iflaviridae, Inoviridae, Iridoviridae, Labyrnaviridae, Large_single_stranded_RNA_satellites, Leviviridae, Lipothrixviridae, Luteoviridae, Malacoherpesviridae, Marnaviridae, Marseillevirusviridae, Microviridae, Mimiviridae, Mononegavirales, Myoviridae, Nanoviridae, Narnaviridae, Nidovirales, Nimaviridae, Nodaviridae, Nudivirus, Ophioviridae, Orthomyxoviridae, Ourmiavirus, Papillomaviridae, Paramyxoviridae, Partitiviridae, Parvoviridae, Phycodnaviridae, Picobirnaviridae, Picornavirales, Picornaviridae, Plasmaviridae, Podoviridae, Polemovirus, Polydnaviridae, Polyomaviridae, Potyviridae, Poxyiridae, Reoviridae, Retroviridae, Rhabdoviridae, Roniviridae, Rudiviridae, Salterprovirus, Secoviridae, Single_stranded_DNA_satellites, Single_stranded_RNA_satellites, Siphoviridae, Sobemovirus, Tectiviridae, Tenuivirus, Tetraviridae, Tobacco_necrosis_satellite_virus-like, Togaviridae, Tombusviridae, Totiviridae, Tymovirales, Tymoviridae, Umbravirus, Varicosavirus, Virgaviridae, environmental_samples, unclassified_archaeal_dsDNA_viruses, unclassified_archaeal_viruses, unclassified_bacteriophages, unclassified_dsDNA_phages, unclassified_dsDNA_viruses, unclassified_dsRNA_viruses, unclassified_ssDNA_viruses, unclassified_ssRNA_negative-strand_viruses, unclassified_ssRNA_positive-strand_viruses, unclassified_dsRNA_viruses, unclassified_virophages, unclassified_viruses

### Archaea:

Acidilobaceae, Aciduliprofundum, Archaeoglobaceae, Candidatus_Haloredivivus, Candidatus_Methanoregula, Candidatus_Methanosphaerula, Cenarchaeaceae, Desulfurococcaceae, Ferroplasmaceae, Fervidicoccaceae, Halobacteriaceae, Korarchaeum, Methanobacteriaceae, Methanocaldococcaceae, Methanocellaceae, Methanococcaceae, Methanocorpusculaceae, Methanomas siliicoccus, Methanomicrobiaceae, Methanopyraceae, Methanoregulaceae, Methanosaetaceae, Methanosarcinaceae, Methanospirillaceae, Methanothermaceae, Nanoarchaeum, Nitrosopumilaceae, Nitrososphaeraceae, Picrophilaceae, Pyrodictiaceae, Sulfolobaceae, Thermococcaceae, Thermofilaceae, Thermoplasmataceae, Thermoproteaceae, environmental_samples, unclassified_Archaea

### Fungi:

Agaricaceae, Ajellomycetaceae, Arthrodermataceae, Ascosphaeraceae, Auriculariaceae, Blastocladiaceae, Botryosphaeriaceae, Ceratobasidiaceae, Chaetomiaceae, Clavicipitaceae, Coniophoraceae, Cordycipitaceae, Coriolaceae, Corticiaceae, Cryphonectriaceae, Culicosporidae, Dacrymycetaceae, Davidiellaceae, Debaryomycetaceae, Dermateaceae, Dipodascaceae, Dothioraceae, Dubosqiidae, Enterocytozoonidae, Erysiphaceae, Ganodermataceae, Glomeraceae, Glomerellaceae, Gnomoniaceae, Harpochytriaceae, Helotiaceae, Herpotrichiellaceae, Hymenochaetaceae, Hypocreaceae, Lasiosphaeriaceae, Legeriomycetaceae, Leotiomycetes, Leptosphaeriaceae, Magnaporthaceae, Malasseziaceae, Marasmiaceae, Metschnikowiaceae, Microbotryaceae, Microsporidia, Mixiaceae, Monoblepharidaceae, Mortierellaceae, Mucoraceae, Mycosphaerellaceae, Nectriaceae, Nosematidae, Omphalotaceae, Onygenaceae, Ophiostomataceae, Orbiliaceae, Peltigeraceae, Phaeosphaeriaceae, Phaffomycetaceae, Phakopsoraceae, Pichiaceae, Plectosphaerellaceae, Pleistophoridae, Pleosporaceae, Pleurotaceae, Pneumocystidaceae, Polyporaceae, Psathyrellaceae, Pucciniaceae, Punctulariaceae, Rhizophydiaceae, Rhizophydiales, Rhodosporidium, Saccharomycetaceae, Saccharomycetales, Saccharomycodaceae, Schizophyllaceae, Schizosaccharomycetaceae, Sclerotiniaceae, Sebacinaceae, Selaginellaceae, Sordariaceae, Spizellomycetaceae, Stereaceae, Taphrinaceae, Taphrinomycotina, Tilletiaceae, Tremellaceae, Trichocomaceae, Tricholomataceae, Tuberaceae, Unikaryonidae, Ustilaginaceae, Wallemiales, Xylariaceae, mitosporic_Ascomycota, mitosporic_Onygenales, mitosporic_Saccharomycetales, mitosporic_Sporidiobolales, mitosporic_Tremellales, unclassified_Fungi, unclassified_Pleosporales

### Protozoa:

Amoebozoa, Apusomonadidae, Babesiidae, Blastocystidae, Capsaspora, Codonosigidae, Cryptomonadaceae, Cryptosporidiidae, Dictyosteliidae, Eimeriidae, Gregarimidae, Hemiselmidaceae, Hexamitidae, Lecudimidae, Monodopsidaceae, Ophryoglenina, Oxytrichidae, Parameciidae, Pelagomonadales, Perkinsidae, Peronosporaceae, Plasmodiidae, Pythiaceae, Saccammimidae, Salpingoecidae, Saprolegniaceae, Sarcocystidae, Tetrahymenidae, Theileriidae, Trichomonadidae, Trypanosomatidae

In some embodiments, the oligonucleotide probes herein described can be provided as a part of systems to perform any assay, including any of the assays described herein. The systems can be provided in the form of arrays or kits of parts. An array, sometimes referred to as a “microarray”, can include any one, two or three dimensional arrangement of addressable regions bearing a particular molecule associated to that region. Usually, the characteristic feature size is micrometers.

In some embodiments, the system can comprise at least two oligonucleotide probes selected for detection of one or more target groups. In those embodiments, the detection can be performed by at least two oligonucleotide probes in combination with other probes, and in particular three or more oligonucleotide probes herein described.

In some embodiments, the system can comprise five or more oligonucleotide probes herein described. In particular, in some embodiments, a system for detection of at least one target in a target group can comprise at least five oligonucleotide probes, having sequence selected from the group consisting of SEQ ID NO's 1-133,263, and wherein at least one target is a microorganism. In some embodiments, the system can comprise five or more oligonucleotide probes herein described. In particular, in some embodiments, a system for detection of at least one target in a target group can comprise at least five oligonucleotide probes, having sequence selected from the group consisting of SEQ ID NO's 133,264-534,156, and wherein at least one target is a microorganism. In some of those embodiments the target groups can comprise the target group exemplified in Example 10 and Example 11 and Example 16.

In other embodiments, oligonucleotide probes can be selected to detect more than one target and in particular more than one target within a target group. For example, targets for detection can comprise two or more selected from a flu virus, a non-flu virus, a virus, and a bacterium, a fungus, a species of protozoa, and an archaeon.

In some embodiments, oligonucleotide probes can be arranged in an array for detection of targets in a target group. In some of those embodiments, the array can comprise a plurality of oligonucleotide probes wherein: at least one of the oligonucleotide probes comprises a sequence selected from the group consisting of SEQ ID NO. 1-133,263. In some of those embodiments, the detection can occur in combination with at least four other oligonucleotide probes selected from the group consisting of SEQ ID NO's 1-133,263, and wherein said target is a microorganism. In some embodiments, oligonucleotide probes can be arranged in an array for detection of targets in a target group. In some of those embodiments, the array can comprise a plurality of oligonucleotide probes wherein: at least one of the oligonucleotide probes comprises a sequence selected from the group consisting of SEQ ID NO. 133,264-534,156. In some of those embodiments, the detection can occur in combination with at least four other oligonucleotide probes selected from the group consisting of SEQ ID NO's 133,264-534,156, and wherein said target is a microorganism.

Further embodiments of the present disclosure also provide: 1) methods of classifying an oligonucleotide probe sequence as detected or undetected in a biological sample; 2) methods of predicting the conditional probability of detecting a probe sequence, given the presence of a target of known nucleotide sequence in a biological sample; 3) methods of predicting likelihood of presence of a target of known nucleotide sequence in a biological sample; 4) selection methods for selecting, from a list of candidate target sequences of known nucleotide sequence, a target sequence most likely to be present in a biological sample; and 5) selection methods for selecting, from a list of candidates, a set of targets whose presence in a biological sample would collectively provide the best explanation for observed detected and undetected probes on an array.

In several embodiments, microarrays are constructed by synthesizing oligonucleotide molecules (denoted henceforth as “oligos”) with the required probe sequences directly upon a solid glass or silica substrate. In other embodiments, oligos are synthesized in a separate process, and then adhered to the substrate. Regardless of the technology used to produce the oligos, an array is partitioned into regions called “features”, each of which is assigned a single known probe sequence. Array construction results in the placement of a large number (on the order of 10⁵ to 10⁷) of identical oligos, all having the assigned probe sequence, within each feature.

In some embodiments a detection microarray for targeting clinically relevant pathogens in a cost effective format is described. The microarray can comprise any number of probes. For example, a microarray can comprise a few probes (i.e. 4 or more), thousands, tens of thousands, hundreds of thousands, or more than hundreds of thousands of probes. In some embodiments the array can comprise probes from families known to infect vertebrates. A skilled person will be able to identify a desired number of probes comprised in an array based on the number and type of target groups to be detected, the features of the oligonucleotide probes and corresponding targets to be included in the array and additional parameters identifiable by a skilled person upon reading of the present disclosure.

In particular, in an exemplary embodiment, complete viral and bacterial genome/segment/plasmid sequences can be gathered and organized by family and regions specific to a family can be identified. From these regions, candidate probes can be identified by base length (50-65 bases), Tm, entropy, GC %, and other thermodynamic and sequence features and desired parameter ranges can be relaxed as needed and candidate probes can be clustered and ranked and uniqueness can be calculated according embodiments herein described. In some embodiments, the base length of candidate probes is shorter than 50 bases, for example 40-49 bases, if no acceptable probes larger than 50 could be found for a target or to adapt the parameters of desired array platforms, such as a maximum probe length of 60 bases for some Agilent® arrays.

In several embodiments, negative control probes having randomly generated sequences are incorporated into the array design. The length and percent GC content distributions of the negative control probe sequences are chosen for each array design to be similar to that of the microbial target probe sequences. Between 1,000 and 10,000 negative control probes are included in each array design. The presence of negative control probes allows estimation of the expected distribution of intensities for probes that have no significant similarity to any target DNA sequence in a biological sample. The method disclosed below for classification of probe sequences as detected or undetected requires the presence of negative control probes. In some embodiments, positive controls are incorporated into the array design. Positive controls can be designed to bind to genomic DNA from an organism, which may be added to a sample for use as an internal quantitation standard. Positive controls can include perfect match probes and probes with a desired range of mismatches, such as 1-9 targeted mismatches. In one exemplary embodiment of this disclosure (v5), probes designed to bind to DNA of Thermotoga maritime were generated and synthesized.

In all embodiments, probe intensity data is generated for each biological sample to be analyzed, according to one of several protocols in common use in the field of this invention. In a typical embodiment, fluorescently labeled target DNA synthesized from templates extracted from a biological sample is incubated for several hours on an array comprising a plurality of probes, to allow for hybridization of target DNA to any probes of the array having sequences similar to those of the target DNA. This procedure produces a variable number of target-probe hybridization products for each probe sequence. Following the hybridization step, the array is washed to remove unhybridized target DNA. A standard microarray scanner is then used to measure an aggregate fluorescence intensity value for each feature on the array. The intensity measured for each feature increases according to the number of target-probe hybridization products involving probes of the sequence assigned to that feature.

In several embodiments of the present disclosure, a method for classifying a target oligonucleotide probe sequence as detected or undetected in a biological sample is provided. The method is as follows: a minimum threshold intensity is determined for each array, as some percentile of the observed distribution of intensities for the negative control probes. Typically the 99^{th} percentile is used, but other values may be selected at the experimenter's discretion. The target probe sequence is then classified as detected if its associated feature intensity exceeds the threshold intensity, and as undetected if not. In several embodiments, this classification determines the value of a binary response variable Yᵢ used in further analysis: 1 if probe i is detected and 0 if not.

Further embodiments provide methods of estimating the conditional detection probability for a particular probe sequence, given the presence of some target of known nucleotide sequence in a biological sample analyzed by a microarray. These methods are based on statistical models for the probability of classifying a probe sequence as detected in a sample, as a function of the nucleotide sequences of the probe itself and of the “most similar” portion of the target sequence. The “most similar” portion of the target sequence is identified by performing a BLAST search, using the probe and target as query and subject sequences respectively, and choosing the target subsequence (if any) having the highest-scoring gap-free alignment. If BLAST finds no alignments exceeding some minimum score threshold, the probe is considered to have no significant similarity to the target sequence; in this case the detection probability is estimated as a function of the probe sequence only.

Estimates of detection probability require choosing a statistical model, and performing a calibration step once for each microarray platform to estimate the parameters of the model. In one embodiment, the model contains four predictor covariates, three of which are determined from the highest-scoring BLAST alignment of probe i to target j. These include the BLAST bit score Bᵢⱼ, and the position Qᵢⱼ of the start of the alignment within the probe sequence. Both of these variables are obtained directly from the BLAST results. The third covariate is an approximate predicted melting temperature Tᵢⱼ, computed from the aligned nucleotides according to the formula Tᵢⱼ=69.4° C.+(41.0 N_{GC}−600.0)/L, where L is the length of the alignment and N_{GC} is the number of G and C nucleotides that are aligned to their complements. The fourth covariate, Sᵢ, depends on the probe sequence only. Sᵢ is the entropy of the trimer frequency table of the probe sequence, which serves as a measure of sequence complexity. It is obtained from the numbers of occurrences n_{AAA}, n_{AAC}, . . . , n_{TTT} of the 64 possible trimers (3-nucleotide subsequences) within the probe sequence, divided by the total number of trimers, yielding the corresponding frequencies f_{AAA}, . . . , f_{TTT}. The entropy is then given by:

$${S}_{i} = {\sum }_{t : {f}_{t} \neq 0} - {f}_{t} {log}_{2} {f}_{t} \qquad ( 1 )$$

Where, the sum is over the trimers t with fₜ≠0. Applicants have found empirically that the trimer entropy is a good predictor of non-specific hybridization; probes with low entropy (and thus low sequence complexity) resulting from direct or tandem repeats are more likely to give strong detection signals regardless of the target sequence.

A statistical model that estimates the detection probability for probe i, conditional on the presence of target j, is then described in terms of these four covariates by the following equations:

logit(P(Yᵢ=1|target j is present))=a₀+a₁Sᵢ+a₂Tᵢⱼ+a₃Bᵢⱼ+a₄Qᵢⱼ (2)

logit(P(Yᵢ=1|target j is absent))=a₀+a₁Sᵢ (3)

In equations (2) and (3), logit(x)=log [x/(1−x)] is the log-odds transformation function, and Yᵢ is the binary response variable indicating whether probe i was classified as detected. The parameters a₀ through a₄ are determined at calibration time, by performing several array hybridizations to individual targets with known genome sequences, measuring the probe intensities, classifying probes as detected or undetected, computing the covariates for all probes, and then fitting the model parameters by standard logistic regression methods. Given a set of fitted parameters and covariates computed for probe i and target j, the conditional detection probability is described by the following equation:

$$P ( {Y}_{i} = 1 \vert {X}_{j} ) = \frac{1}{1 + {e}^{- ( {a}_{0} + {a}_{1} {S}_{i} + {X}_{j} ( {a}_{2} {T}_{ij} + {a}_{3} {B}_{ij} + {a}_{3} {Q}_{ij} ) )}} \qquad ( 4 )$$

Where, Xⱼ is an indicator variable, with value 1 if target j is present and 0 if not.

Another embodiment of the present disclosure provides an alternative method for predicting conditional detection probabilities. This method is based on a logistic model, with two covariates in place of the four used in the previously described method. The two covariates are the trimer entropy Sᵢ described above, and the free energy ΔGᵢⱼ predicted for the highest-scoring probe-target alignment. The free energy is predicted from the aligned probe and target subsequences, using the nearest-neighbor stacking energy model described in reference 27, with an optional position-specific weight factor. The model is described by the equations:

logit(P(Yᵢ=1|target j is present))=b₀+b₁Sᵢ+b₂ΔGᵢⱼ (5)

logit(P(Yᵢ=1|target j is absent))=b₀+b₁Sᵢ (6)

where b₀, b₁ and b₂ are model parameters to be fitted at calibration time, and other variables are as described previously. In all other respects, this method is the same as the previously described method for estimating detection probabilities. The resulting conditional detection probability is described by the equation:

$$P ( {Y}_{i} = 1 \vert {X}_{j} ) = \frac{1}{1 + {e}^{- ( {b}_{0} + {b}_{1} {S}_{i} + {b}_{2} {X}_{j} Δ {G}_{ij} )}} \qquad ( 7 )$$

Further embodiments provide methods of predicting the likelihood of presence of a particular target, of known nucleotide sequence, in a biological sample. In several embodiments, target DNA from the biological sample is hybridized to an array, fluorescence intensities are measured for each probe sequence, and probe sequences are classified as detected or undetected using one of the methods described above. Let Yᵢ be the binary response variable indicating whether probe i was classified as detected (1) or undetected (O). The probe responses are used to compute a likelihood function, under the assumption that the responses for different probes are conditionally independent of one another, given the presence or absence of specified target j. If Y represents the vector of probe response variables Yᵢ, the likelihood of target j being present in the sample (Xⱼ=1) or absent (Xⱼ=0) given the observed response is given by the equation:

$$L ( {X}_{j } ; Y ) = {\prod }_{i : {Y}_{i} = 1} P ( {Y}_{i} = 1 \vert {X}_{j} ) {\prod }_{i : {Y}_{i} = 0} P ( {Y}_{i} = 0 \vert {X}_{j} ) \qquad ( 8 )$$

where P(Yᵢ=1|Xⱼ) is given by equation (4) or (7), and P(Yᵢ=0|Xⱼ)=1−P(Yᵢ=1|Xⱼ).

In several embodiments, a single target selection method is provided for choosing, from a list of candidate targets of known nucleotide sequence, the target that is most likely to be present in a biological sample. After hybridizing the sample to an array, scanning the array and classifying probe sequences as detected or undetected, the relative likelihoods of target presence versus absence are computed for each candidate target by evaluating the aggregate log-odds score:

$$log \frac{L ( {X}_{j} = 1 ; Y )}{L ( {X}_{j} = 0 ; Y )} = {\sum }_{i : {Y}_{i} = 1} log \frac{P ( {Y}_{i} = 1 \vert {X}_{j} = 1 )}{P ( {Y}_{i} = 1 \vert {X}_{j} = 0 )} + {\sum }_{i : {Y}_{i} = 0} log \frac{P ( {Y}_{i} = 0 \vert {X}_{j} = 1 )}{P ( {Y}_{i} = 0 \vert {X}_{j} = 0 )} \qquad ( 9 )$$

To choose the most likely target, an aggregate log-odds score is computed for each candidate target, and the target with the maximum score is selected.

In several embodiments of the present disclosure, a multiple target selection method is provided to select a combination of targets whose presence in a biological sample would best explain the observed pattern of probe responses on an array hybridized to the sample. The selection method employs a greedy algorithm to find a local maximum for the log-likelihood. The algorithm is initialized by placing all candidate targets in an “unselected” list U and an empty “selected” list S. The following steps are then iterated until the algorithm terminates:
1. Compute the conditional log-odds score for each target jεU:

$${\sum }_{i : {Y}_{i} = 1} log \frac{P ( {Y}_{i} = 1 \vert {X}_{j} = 1 , {X}_{k} = 1 ∀ k ∈ S )}{P ( {Y}_{i} = 1 \vert {X}_{j} = 0 , {X}_{k} = 1 ∀ k ∈ S )} + {\sum }_{i : {Y}_{i} = 0} log \frac{P ( {Y}_{i} = 0 \vert {X}_{j} = 1 , {X}_{k} = 1 ∀ k ∈ S )}{P ( {Y}_{i} = 0 \vert {X}_{j} = 0 , {X}_{k} = 1 ∀ k ∈ S )} \qquad ( 10 )$$

When this step is performed for the first time, the selected list S will be empty, so the computed log-odds score for each target will not be conditioned on the presence of any other targets. Store this “initial” log-odds score for each target, for later display.
2. Choose the target that yields the largest value of the score, remove it from list U, and add it to the selected list S. Store the value of this “final” score for each selected target.
3. Repeat steps 1 and 2 until there is no target in U that yields a positive value for the conditional log-odds score.
   To compute the conditional probabilities in equation (10), the method uses the approximation:

$$P ( {Y}_{i} = 0 \vert X ) \approx {\prod }_{j : {X}_{j} = 1} P ( {Y}_{i} = 0 \vert {X}_{j} = 1 ) \qquad ( 11 )$$

Where, X represents a vector of binary Xₖ values. In other words, it assumes that the probability of obtaining an undetected response for a probe depends only on the set of targets that are assumed to be present, and that it can be estimated by multiplying the probabilities conditioned on the presence of the individual targets. The conditional detection probabilities are given by:

$$P ( {Y}_{i} = 1 \vert X ) \approx 1 - {\prod }_{j : {X}_{j} = 1} P ( {Y}_{i} = 0 \vert {X}_{j} = 1 ) \qquad ( 12 )$$

The output of the multiple target selection method is an ordered series of target genomes predicted to be present, together with of the initial and final scores for each selected target. The initial score is the log-odds from the first iteration; that is, the log-likelihood of the target being present assuming that no other targets are present. The final score for the n^{th} selected target is the log-odds conditional on the presence of the first through the (n−1)^{st} selected targets.

Conditioning on the previously selected targets has the effect of subtracting the contributions from the associated probes from the log-likelihood. Therefore, the multiple target selection algorithm can be visualized as an iterative process that first chooses the target that explains the greatest number of probes with positive detection signals, while minimizing the number of undetected probes that would also be expected to be present; then chooses the target that explains the largest number of probes not already explained by the first target, and so on until as many detected probes as possible are explained.

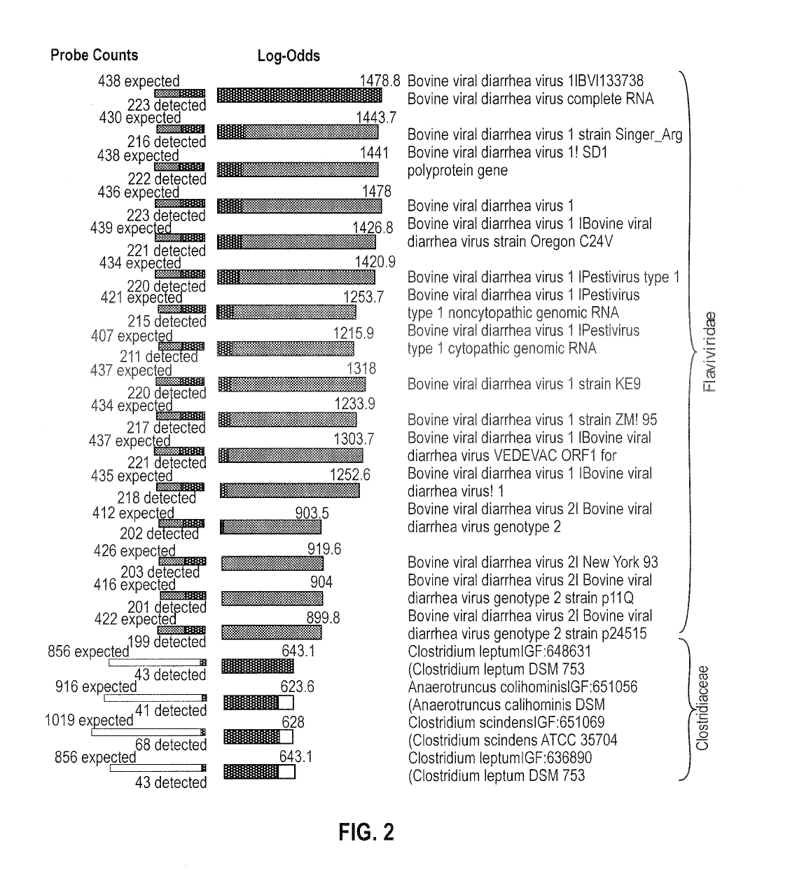

An example of the analysis results is shown in FIG. 2. The right-hand column of bar graphs shows the initial and final log-odds scores for each target genome listed at right. The initial log-odds is the larger of the two scores; thus the lighter and darker-shaded portions represent the initial and final scores respectively. That is, the darker shade on the left part of the bar shows the contribution from a target that cannot be explained by another, more likely target above it, while the lighter shaded part on the right of the bar illustrates that some very similar targets share a number of probes, so that multiple targets may be consistent with the hybridization signals. Targets are grouped by taxonomic family, indicated by the bracket to the side; they are listed within families in decreasing order of final log-odds scores.

The left-hand column of bar graphs shows the expectation (mean) values of the numbers of probes expected to be present given the presence of the corresponding target genome. The larger “expected” score is obtained by summing the conditional detection probabilities for all probes; the smaller “detected” score is derived by limiting this sum to probes that were actually detected. Because probes often cross-hybridize to multiple related genome sequences, the numbers of “expected” and “detected” probes often greatly exceed the number of probes that were actually designed for a given target organism. The probe count bar graphs are designed to provide some additional guidance for interpreting the prediction results.

In some embodiments, detection of a target can be performed by contacting a sample with any of the oligonucleotide probes, systems and array herein described for a time and under condition to allow formation of oligonucleotide probes-target sequences complex in the sample, In particular, the oligonucleotide probes-target sequence complex can provide a detectable signal. In some embodiments, the method can further comprise predicting a target sequence most likely to be present in the sample based on the detectable signal from the oligonucleotide probe-target sequence complex.

The wording “signal” or “labeling signal” as used herein indicates the signal emitted from a label that allows detection of the label, including but not limited to radioactivity, fluorescence, chemiluminescence, production of a compound in outcome of an enzymatic reaction and the like. The terms “label” and “labeled molecule” as used herein as a component of a complex or molecule referring to a molecule capable of detection, including but not limited to radioactive isotopes, fluorophores, chemiluminescent dyes, chromophores, enzymes, enzymes substrates, enzyme cofactors, enzyme inhibitors, dyes, metal ions, nanoparticles, metal sols, ligands (such as biotin, avidin, streptavidin or haptens) and the like. The term “fluorophore” refers to a substance or a portion thereof which is capable of exhibiting fluorescence in a detectable image.

In some embodiments, the target can be a microorganism, the sample can be contacted with at least one of the oligonucleotide probes having a sequence selected from the group consisting of SEQ ID NO. 1-133,263; in combination with at least four other oligonucleotide probes selected from SEQ ID NO's 1-133,263, with oligonucleotide probes presenting a label. In some embodiments, the target can be a microorganism, the sample can be contacted with at least one of the oligonucleotide probes having a sequence selected from the group consisting of SEQ ID NO. 133,264-534,156; in combination with at least four other oligonucleotide probes selected from SEQ ID NO's 133,264-534,156, with oligonucleotide probes presenting a label. In some embodiments, the target can be a microorganism, the sample can be contacted with at least one of the oligonucleotide probes having a sequence selected from the group consisting of SEQ ID NO. 491,463-495,658 and 534,157-661,081; in combination with at least four other oligonucleotide probes selected from SEQ ID NO's 491,463-495,658 and 534,157-661,081, with oligonucleotide probes presenting a label. In some of those embodiments, the target can be detected by contacting the sample with the array and predicting a target sequence most likely to be present in the sample based on one or more corresponding labeling signals according to methods herein described or identifiable by a skilled person upon reading of the present disclosure. In some of those embodiments, the sample can be a biological sample.

In some embodiments, the contacting of the oligonucleotide probes, systems and/or arrays herein described can be performed by hybridizing the sample to the oligonucleotide probes, systems and/or array.

In particular, in some embodiments hybridizing can be performed by incubating fluorescently labeled target DNA synthesized from templates extracted from a biological sample on an array comprising a plurality of probes, to allow for hybridization of target DNA to any probes of the array having sequences similar to those of the target DNA, producing a variable number of target-probe hybridization products for each probe sequence; scanning the array to measure an aggregate fluorescence intensity value.

In some of those embodiments, the intensity can be measured for each feature increases according to the number of target-probe hybridization products involving probes of the sequence assigned to that feature.

In some embodiments the predicting of a target sequence most likely to be present in the biological sample can comprise: classifying an oligonucleotide probe sequence as detected or undetected in a biological sample; predicting likelihood of presence of a target of known nucleotide sequence in a biological sample; and selecting, from a list of candidate target sequences of known nucleotide sequence, a target sequence most likely to be present in a biological sample.

In summary, in accordance with embodiments of the present disclosure, probes were selected to avoid sequences with high levels of similarity to human, bacterial and viral sequences not in the target family; low levels of sequence similarity across families were allowed selectively, on the basis of a statistical model predicting probe intensity from the similarity score, approximate melting temperature and sequence complexity. Favoring more conserved probes within a family enabled us to minimize the total number of probes needed to cover all existing genomes with a high probe density per target, enhancing the capability to identify the species of known organisms and to detect unsequenced or emerging organisms. Strain or subtype identification was not a goal of the MDA discovery probe design, although the ability of MDA v1, v2, v3.3, and v3.4 to discriminate between strains of certain organisms was an unexpected result of combining signals from multiple probes. The goal of the census probes on MDA v3.1 and v3.2 was to discriminate between strains or subtypes, so the combination of signals from both the conserved “discovery” probes and the census probes should reinforce and improve strain discrimination.

In accordance with some embodiments, probes were sufficiently long (50-66 bases) to tolerate some sequence variation (see reference 8), although slightly shorter than the 70-mer probes used on previous arrays (see references 4, 14 and 23) because of the additional synthesis cycles, and therefore cost, of making 70-mers on the NimbleGen platform. Long probes improve hybridization sensitivity and efficiency, alleviate sequence-dependent variation in hybridization, and improve the capability to detect unsequenced microbes. Probes were selected from whole genomes, without regard to gene locations or identities, letting the sequences themselves determine the best signature regions and preclude bias by pre-selection of genes. Applicants designed a version 1 (v1) with 36,000 distinct probe sequences for viruses (at least 15 probes per viral sequence), and then designed a version 2 (v2) that included 170,000 probe sequences for viruses (at least 50 probes/sequence) and 8,000 probe sequences for bacteria (at least 15 probes per sequence), and included the ViroChip v3 (see reference 23) probes for comparison. Applicants designed a version 5 (v5) to contain two sets of probes, a 360K set which included at least 30 probes per target sequence selected from conservation favoring probes, at least 5 probes per target sequence selected from discriminating probes, and Primux k-mer probes, and a 135K set, which included at least 15 conserved probes per target sequence and at least 2 discriminating probes per sequence. Applicates designed a 360K set to represent 5,434 microbial species, 3,111 viral species, 1,967 bacterial species, 126 archaeal species, 94 protozoa species, and 136 fungi species (SEQ ID NOs 133,264-491462 and 495,659-534,156). Applicants designed a 135K set to represent 3,521 microbial species represented with 1,856 viral species, 1,398 bacterial species, 125 archaeal species, 94 protozoa species, and 48 fungi species (SEQ ID NOs 491,463-495,658 and from 534,157-661,081). Arrays were built at NimbleGen using a NimbleGen Array Synthesizer (see reference 19). Applicants hybridized the arrays to a number of samples, including clinical fecal, sputum, and serum samples. In blinded clinical samples containing multiple viruses and bacteria and in known (spiked) mixtures of DNA and RNA viruses, the MDA has been able to detect viruses and bacteria as confirmed by PCR or culture.

In addition, a statistical method has been described that is based on likelihood maximization within a Bayesian network model. It incorporates a probabilistic model of DNA hybridization based on probe-target similarity scores and probe sequence complexity, with parameters fitted to experimental data from pure viral and bacterial samples with sequenced genomes. To accurately determine the organism(s) responsible for a given array result, the pattern of both present and absent probe signals is taken into account (see reference 8).

In some embodiments, the microarray and statistical analysis method described herein can detect viral and bacterial sequences from single DNA and RNA viruses and mixtures thereof, various clinical samples, and blinded cell culture samples. In particular, in some embodiments, results from clinical samples can be validated, for example by using PCR.

For example, the MDA v.2 as described herein can be applied to problems in target detection, with particular reference to viral and bacterial detection, from pure or complex environmental or clinical samples and can be particularly useful to widen a scope of search for microbial identification when specific PCR fails, as well as to identify co-infecting organisms. In some embodiments, the ability of the microarray to detect viral and bacterial sequences and to detect various clinical samples can be functional to probe density and phylogenetic representation of viral and bacterial sequenced genomes. In particular, in some embodiments, arrays can be provided that allow detection of viral and bacterial sequences with a higher and larger phylogenetic representation in comparison with certain array designs identifiable by a skilled person.

In some embodiments a method to obtain a plurality of oligonucleotide probes for detection of targets of a target group is provided, the method comprising: identifying group-specific candidate probes from an initial genomic collection by eliminating from the initial collection regions with matches to non-group targets above a match threshold and by selecting regions satisfying probe characteristics, said probe characteristics including at least one criterion selected from length, Tₘ, GC %, maximum homopolymer length, homodimer free energy prediction, hairpin free energy prediction, probe-target free energy prediction, and minimum trimer frequency entropy condition; ranking the group-specific candidate probes in decreasing order of number of targets of the target group represented by each group-specific candidate probe; and selecting probes from the ranked group-specific candidate probes.

In some embodiments, a method as described in paragraph 00121 is provided, wherein selecting probes from the ranked group-specific candidate probes comprises, for each target, selecting the most conserved or least conserved probes representing that target until each target genome is represented by a predetermined number of probes.

In some embodiments, a method as described in paragraph 00121 is provided, and the method further comprises clustering together candidate probes sharing at least 85% identity and selecting the longest sequence from each cluster as a target for probe design.

In some embodiments, a method as described in paragraph 00121 is provided, wherein at least one criterion is relaxed to obtain at least a minimum number of candidate probes for each target.

In some embodiments, a method as described in paragraph 00121 is provided, wherein a target is represented if a candidate probe matches with at least 85% sequence similarity over the total candidate probe length and a perfectly matching subsequence of at least 29 contiguous bases spans the middle of the probe.

In some embodiments, a method as described in paragraph 00121 is provided, wherein the group is selected between a viral family, a bacterial family, a viral sequence group classified under a taxonomic node other than family, and a bacterial sequence group classified under a taxonomic node other than family.

In some embodiments, a method as described in paragraph 00121 and 00120 is provided, wherein the group is a viral family and the probes are at least 50 per target.

In some embodiments, a method as described in paragraphs 00121 and 00120 is provided, wherein the group is a bacterial family and the probes are at least 15 per target.

In some embodiments, a method as described in paragraph 00121 is provided, wherein the probes are at least 50 bases long.

In some embodiments, a method as described in paragraphs 00121 and 00120 is provided, wherein group-specific regions are identified for probe selection that do not have a match of an oligonucleotide of x or more nucleotides long with sequences not part of the group, x being an integer.

In some embodiments, a method as described in paragraphs 00121 and 00120 and 00116 is provided, where the group is a viral family or a bacterial family and where x=17 nucleotides for a viral family and x=25 nucleotides for a bacterial family.

In some embodiments a plurality of oligonucleotide probes for detection of targets of a target group is described, the plurality obtained the method described in paragraphs 00121.

In some embodiments an array comprising the plurality of oligonucleotide probes as described in paragraph 00132 is described.

In some embodiments an array as described in paragraph 00133 is described, wherein the number of probes of the array differs according to the target.

In some embodiments, a method of classifying an oligonucleotide probe sequence as detected or undetected in a biological sample is provided, the method comprising: incubating fluorescently labeled target DNA synthesized from templates extracted from a biological sample on an array comprising a plurality of probes, to allow for hybridization of target DNA to any probes of the array having sequences similar to those of the target DNA, producing a variable number of target-probe hybridization products for each probe sequence; scanning the array to measure an aggregate fluorescence intensity value for each feature comprising a set of target-probe hybridization products having probes of the same sequence; calculating the distribution of feature intensity values for target-probe hybridization products by way of negative control probes with randomly generated sequences, and setting a minimum detection threshold for the array; and comparing the observed feature intensity value for each probe sequence with the minimum detection threshold determined for the array, to classify each probe sequence on the array as either detected or undetected in the biological sample.

In some embodiments, a method of predicting likelihood of presence of a target of known nucleotide sequence in a biological sample is provided, the method comprising: applying the method as described in paragraph 127 to classify probe sequences on an array as detected or undetected in the sample; estimating, for each detected probe sequence: i) a probability of observing the probe sequence as detected conditioned on presence of the target of known nucleotide sequence; ii) a probability of observing the probe sequence as detected conditioned on absence of the target of known nucleotide sequence; and iii) the detection log-odds, defined as the ratio of i) and ii); estimating, for each undetected probe sequence: iv) a probability of observing the probe sequence as undetected conditioned on presence of the target of known nucleotide sequence; v) a probability of observing the probe sequence as undetected conditioned on absence of the target of known nucleotide sequence; and vi) the nondetection log-odds, defined as the ratio of iv) and v); summing detection and nondetection log-odds values over the probes on the array to form an aggregate log-odds score for presence versus absence of the target of known nucleotide sequence, conditional on the observed detected and undetected probes; and based on the aggregate log-odds score, providing a prediction of the presence of at least one said target of known nucleotide sequence in the biological sample.

In some embodiments, a selection method for selecting, from a list of candidate target sequences of known nucleotide sequence, a target sequence most likely to be present in a biological sample is provided, the selection method comprising: applying the method as described in paragraph 00136 to each of the candidate target sequences, and choosing the target sequence that yields the maximum aggregate log-odds score.

In some embodiments, a method as described in paragraph 00136 is provided, wherein i) is estimated by performing a BLAST alignment of the probe sequence and target of known nucleotide sequence, and evaluating a logistic probability density function with BLAST bit score, predicted melting temperature, and position of an aligned portion of the target of known nucleotide sequence within the probe sequence as covariates, and coefficients fitted to data from arrays hybridized to targets of known nucleotide sequence.

In some embodiments a method as described in paragraph 00136 is provided, wherein i) is estimated by performing a BLAST alignment of the probe sequence and target of known nucleotide sequence, and evaluating a logistic probability density function with predicted free energy of the probe-target hybridization as covariate, and coefficients fitted to data from arrays hybridized to targets of known nucleotide sequence.

In some embodiments a method as described in paragraph 00136 is provided, wherein ii) is estimated as a logistic function of probe sequence entropy, computed from a frequency distribution of nucleotide trimers within the probe sequence.

In some embodiments a selection method for selecting, from a list of candidates, a set of targets whose presence in a biological sample would collectively provide the best explanation for observed detected and undetected probes on an array is described, the method comprising: a) applying the method as described in paragraph 00137 wherein to identify the target most likely to be present in the sample; b) removing the identified target from the list of candidates and adding the identified target to the “selected” list; c) repeating the method as described in paragraph 00137 for the remaining candidates, wherein: c1) estimation of i), ii) and iii) is replaced with estimation of: i′) a probability of observing the probe sequence as detected conditioned on presence of the candidate target and presence of targets in the list of selected targets; ii′) a probability of observing the probe sequence as detected conditioned on absence of the candidate target and presence of targets in the list of selected targets; and iii′) the detection log-odds, defined as the ratio of i′) and ii′); c2) estimation of iv), v) and vi) is replaced with estimation of: iv′) a probability of observing the probe sequence as undetected conditioned on presence of the candidate target and presence of targets in the list of selected targets; v′) a probability of observing the probe sequence as undetected conditioned on absence of the candidate target and presence of the targets in the list of selected targets; and vi′) the nondetection log-odds, defined as the ratio of iv′) and v′); c3) the detection and nondetection log-odds values are summed over the probes on the array to form a conditional log-odds score for presence versus absence of the candidate target, conditioned on the observed detected and undetected probes and on the presence of the targets in the list of selected targets; d) choosing the candidate target yielding the maximum conditional log-odds score, removing it from the candidate list, and adding it to the list of selected targets; and e) repeating c) and d) until the conditional log-odds scores for all remaining candidate targets are less than zero. In some embodiments of the present disclosure, a kit of parts is described. The kit of parts can comprise components suitable for preparing an array, including but not limited to a solid glass and/or silica substrate on which oligonucleotide probes can be arranged, primers, and/or reagents suitable for synthesizing oligonucleotide probes according to the present disclosure.

In some embodiments, the kit further comprises a set of instructions, the instructions providing a method to prepare an array according to the present disclosure. In particular, the instructions can provide a method to synthesize oligonucleotide probes for detecting targets in a target group and/or a species in a sample; a method to provide an array comprising the oligonucleotide probes; and a method to use the array for detection of a target, given a particular target group.

In a kit of parts, the oligonucleotide probes and other reagents to perform the assay can be comprised in the kit independently. The oligonucleotide probes can be included in one or more compositions, and each oligonucleotide probe can be in a composition together with a suitable vehicle.

Additional components can include labeled molecules and in particular, labeled polynucleotides, labeled antibodies, labels, microfluidic chip, reference standards, and additional components identifiable by a skilled person upon reading of the present disclosure.

In some embodiments, detection of a oligonucleotide probes can be carried either via fluorescent based readouts, in which the labeled antibody is labeled with fluorophore, which includes, but not exhaustively, small molecular dyes, protein chromophores, quantum dots, and gold nanoparticles. Additional techniques are identifiable by a skilled person upon reading of the present disclosure and will not be further discussed in detail.

In particular, the components of the kit can be provided, with suitable instructions and other necessary reagents, in order to perform the methods here described. The kit will normally contain the compositions in separate containers. Instructions, for example written or audio instructions, on paper or electronic support such as tapes or CD-ROMs, for carrying out the assay, will usually be included in the kit. The kit can also contain, depending on the particular method used, other packaged reagents and materials (i.e. wash buffers and the like).

In some embodiments, the instructions provide a method to directly synthesize oligonucleotide probes on the array. In other embodiments the instructions comprise steps to attach synthesized oligonucleotide probes to the array.

In an embodiment, steps in the methods to obtain a plurality of oligonucleotides of the present disclosure can be written in a variety of computer programming and scripting languages. In particular, the sequences of the oligonucleotides and the executable steps according to the methods and algorithms of the disclosure can be stored on a physical medium, a computer, or on a computer readable medium. All the software programs were developed, tested and installed on desktop PCs and multi-node clusters with Intel processors running the Linux operating system. The various steps can be performed in multiple-processor mode or single-processor mode. All programs should also be able to run with minimal modification on most PCs and clusters. The steps outlined in FIGS. 1A, 1B and 15 can be written as modules configured to perform the task. Additional steps to further optimize the method of the present disclosure can be written as additional modules to be performed in sequence or concurrently with other modules of the method.

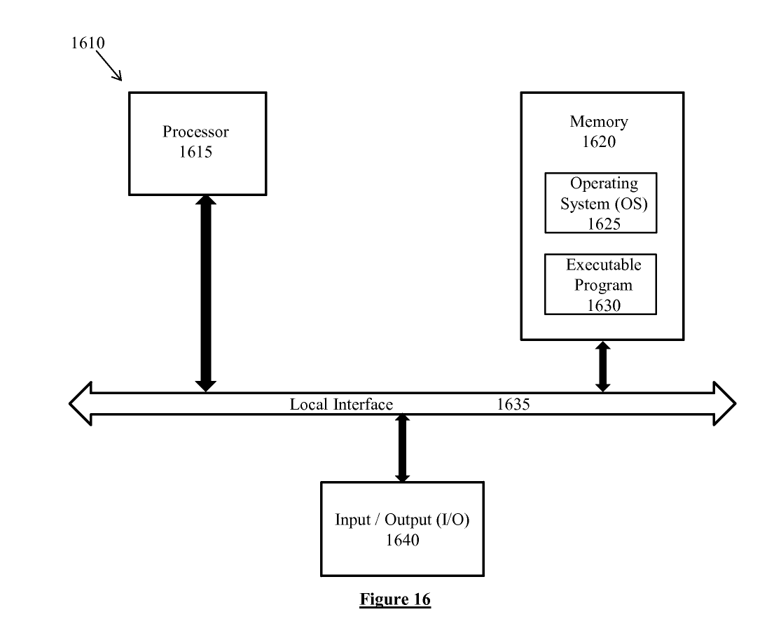

FIG. 16 shows a computer system 1610 that may be used to implement the Method of the present disclosure. It should be understood that certain elements may be additionally incorporated into computer system 1610 and that the figure only shows certain basic elements (illustrated in the form of functional blocks). These functional blocks include a processor 1615, memory 1620, and one or more input and/or output (I/O) devices 1640 (or peripherals) that are communicatively coupled via a local interface 1635. The local interface 1635 can be, for example, metal tracks on a printed circuit board, or any other forms of wired, wireless, and/or optical connection media. Furthermore, the local interface 1635 is a symbolic representation of several elements such as controllers, buffers (caches), drivers, repeaters, and receivers that are generally directed at providing address, control, and/or data connections between multiple elements.

The processor 1615 is a hardware device for executing software, more particularly, software stored in memory 1620. The processor 1615 can be any commercially available processor or a custom-built device. Examples of suitable commercially available microprocessors include processors manufactured by companies such as Intel, AMD, and Motorola.

The memory 1620 can include any type of one or more volatile memory elements (e.g., random access memory (RAM, such as DRAM, SRAM, SDRAM, etc.)) and nonvolatile memory elements (e.g., ROM, hard drive, tape, CDROM, etc.). The memory elements may incorporate electronic, magnetic, optical, and/or other types of storage technology. It must be understood that the memory 1620 can be implemented as a single device or as a number of devices arranged in a distributed structure, wherein various memory components are situated remote from one another, but each accessible, directly or indirectly, by the processor 1615.

The software in memory 1620 may include one or more separate programs, each of which comprises an ordered listing of executable instructions for implementing logical functions. In the example of FIG. 16, the software in the memory 1620 includes an executable program 1630 that can be executed perform the method of the present disclosure. Memory 1620 further includes a suitable operating system (OS) 1625. The OS 1625 can be an operating system that is used in various types of commercially-available devices such as, for example, a personal computer running a Windows® OS, an Apple® product running an Apple-related OS, or an Android OS running in a smart phone. The operating system 1625 essentially controls the execution of executable program 1630 and also the execution of other computer programs, such as those providing scheduling, input-output control, file and data management, memory management, and communication control and related services.

Executable program 1630 is a source program, executable program (object code), script, or any other entity comprising a set of instructions to be executed in order to perform a functionality. When a source program, then the program may be translated via a compiler, assembler, interpreter, or the like, and may or may not also be included within the memory 1620, so as to operate properly in connection with the OS 1625.

The I/O devices 1640 may include input devices, for example but not limited to, a keyboard, mouse, scanner, microphone, etc. Furthermore, the I/O devices 1640 may also include output devices, for example but not limited to, a printer and/or a display. Finally, the I/O devices 1640 may further include devices that communicate both inputs and outputs, for instance but not limited to, a modulator/demodulator (modem; for accessing another device, system, or network), a radio frequency (RF) or other transceiver, a telephonic interface, a bridge, a router, etc.

If the computer system 1610 is a PC, workstation, smartdevice, or the like, the software in the memory 1620 may further include a basic input output system (BIOS) (omitted for simplicity). The BIOS is a set of essential software routines that initialize and test hardware at startup, start the OS 1625, and support the transfer of data among the hardware devices. The BIOS is stored in ROM so that the BIOS can be executed when the computer system 1610 is activated.

When the computer system 1610 is in operation, the processor 1615 is configured to execute software stored within the memory 1620, to communicate data to and from the memory 1620, and to generally control operations of the computer system 1610 pursuant to the software. Method of the present disclosureing and the OS 1625 are read by the processor 1615, perhaps buffered within the processor 1615, and then executed.

When the audio data spread spectrum embedding and detection system is implemented in software, as is shown in Figure. 16, it should be noted that the computer-executable steps of the method of the present disclosure can be stored on any computer readable storage medium for use by, or in connection with, any computer related system or method. In the context of this document, a computer readable storage medium is an electronic, magnetic, optical, or other physical device or means that can contain or store a computer program for use by, or in connection with, a computer related system or method.

Several steps of the method according to the present disclosure can be embodied in any computer-readable storage medium for use by or in connection with an instruction execution system, apparatus, or device, such as a computer-based system, processor-containing system, or other system that can fetch the instructions from the instruction execution system, apparatus, or device and execute the instructions. In the context of this document, a “computer-readable storage medium” can be any means that can store, communicate, propagate, or transport the program for use by or in connection with the instruction execution system, apparatus, or device. The computer readable storage medium can be, for example but not limited to, an electronic, magnetic, optical, electromagnetic, infrared, or semiconductor system, apparatus, or device. More specific examples (a non-exhaustive list) of the computer-readable storage medium would include the following: a portable computer diskette, a random access memory (RAM), a read-only memory (ROM), an erasable programmable read-only memory (EPROM, EEPROM, or Flash memory) an optical disk such as a DVD or a CD.

In an alternative embodiment, where some or all of the steps of a method of the present disclosure to the present disclosure are implemented in hardware, the audio data spread spectrum embedding and detection system can implemented with any one, or a combination, of the following technologies, which are each well known in the art: a discrete logic circuit(s) having logic gates for implementing logic functions upon data signals, an application specific integrated circuit (ASIC) having appropriate combinational logic gates, a programmable gate array(s) (PGA), a field programmable gate array (FPGA), etc.

### EXAMPLES

The arrays, methods and systems of several embodiments herein described are further illustrated in the following examples, which are provided by way of illustration and are not intended to be limiting. A person skilled in the art will appreciate the applicability of the features described in detail for methods.

### Example 1

### Sample Preparation and Microarray Hybridization

DNA microarrays were synthesized using the NimbleGen Maskless Array Synthesizer at Lawrence Livermore National Laboratory as described in reference 8. Adenovirus type 7 strain Gomen (Adenoviridae), respiratory syncytial virus (RSV) strain Long (Paramyxoviridae), respiratory syncytial virus strain B1, bluetongue virus (BTV) type 2 (Reoviridae) and bovine viral diarrhea virus (BVDV) strain Singer (Flaviviridae) were purchased from the National Veterinary lab and grown at LLNL. Purified DNA from human herpesvirus 6B (HHV6B) (Herpesviridae) and vaccinia virus strain Lister (Poxyiridae) were purchased from Advanced Biotechnologies (Maryland, Va.). Eleven blinded viral culture samples were received from Dr. Robert Tesh's lab at University of Texas Medical Branch at Galveston (UTMB). The viral cultures were sent to LLNL in the presence of Trizol reagent.

After treatment with Trizol reagent, RNA from cells was precipitated with isopropanol and washed with 70% ethanol. The RNA pellet was dried and reconstituted with RNase free water. 1 μg of RNA was transcribed into double-strand cDNA with random hexamers using Superscript™ double-stranded cDNA synthesis kit from Invitrogen (Carlsbad, Calif.). The DNA or cDNA was labeled using Cy-3 labeled nonamers from Trilink Biotechnologies and 4 μg of labeled sample was hybridized to the microarray for 16 hours as previously described (see reference 8). Clinical samples that had been extracted and partially purified using Round A and Round B protocols (see reference 23) were obtained from Dr. Joseph DeRisi's laboratory at University of California, San Francisco (UCSF). The samples were amplified for an additional 15 cycles to incorporate aminoallyl-dUTP and labeled with Cy3NHS ester (GE Healthcare (Piscataway, N.J.). The labeled samples were hybridized to NimbleGen arrays.

### Example 2

### Testing on Pure and Mixed Samples of Known Viruses for Array v1

Several of the viruses of Example 1 (adenovirus type 7, RSV, and BVDV) were hybridized on array v1 in single virus hybridization experiments and each was detected by array v1 (data not shown). Several mixtures of both RNA and DNA viruses were also tested (Table 6). PCR primers used to detect or confirm various samples before or after testing samples on the arrays of the present disclosure are provided in Table 9.

TABLE 6
Results of initial tests on array v1.

| Mixture tested | Detected | Additionally detected |
|---|---|---|
| Adenoviral type 7 strain | Yes | Human endogenous |
| Gomen | | retrovirus |
| Respiratory syncytial virus | Yes | K113 |
| strain Long | | |
| Bovine viral diarrhea type 1 | Yes | Leek yellow stripe |
| strain Singer | | potyvirus |
| Respiratory syncytial virus | Yes | none |
| strain B1 | | |
| Bluetongue virus type 2 | Yes | |
| | (segments | |
| | 2, 6, 8, 9, 10) | |
| Human herpesvirus 6B | Yes | Human endogenous |
| | | retrovirus |
| Vaccinia virus strain Lister | Yes | K113 |
| Respiratory syncytial virus | Yes | Influenza A segment 8 |
| strain B1 | | |
| Bluetongue virus type 2 | Yes | |
| | (segments | |
| | 2, 6, 7, 8, 9, 10) | |

All spiked species from Table 6 were detected in the mixture, including most of the segments of BTV. Strain discrimination was not expected, since probes were designed from regions conserved within viral families. Nevertheless, the highest scoring targets in the single virus experiments with adenovirus, BVDV, vaccinia and HHV 6B were in fact the strains hybridized to the arrays. Human endogenous retrovirus K113 was also detected in two of the three mixtures, possibly derived from host cell DNA.

For three particular samples tested, spiked strain identities were compared with those predicted by analyzing either 1) only the LLNL probes versus 2) analyzing only the Virochip probes that were also included on the MDA. The LLNL probes identified the correct Gomen strain of human adenovirus type 7 while the Virochip probes identified the correct species but the incorrect NHRC 1315 strain. In another example, when RSV Long group A (an unsequenced strain) was hybridized to the array, the related RSV strain ATCC VR-26 was predicted by MDA probes, but the Virochip probes failed to detect any RSV strain. For the detection of BVD Singer strain, both LLNL and Virochip probes were able to predict the exact strain hybridized.

### Example 3

### PCR to Confirm Microarray Results

Clinical samples from the DeRisi laboratory (Example 1) were tested by PCR to confirm the microarray results (Example 2). PCR primers were designed using either the KPATH system (see reference 20) or based on the probes that gave a positive signal for the organism identified as present, and the primer sequences are proved as supplementary information. PCR primers were synthesized by Biosearch Technologies Inc (Novato, Calif.). 1 μL of Round B material was re-amplified for 25 cycles and 2 μL of the PCR product was used in a subsequent PCR reaction containing Platinum Taq polymerase (Invitrogen), 200 mM primers for 35 cycles. The PCR condition is as follows: 96° C., 17 sec, 60° C., 30 sec and 72° C., 40 sec. The PCR products were visualized by running on a 3% agarose gel in the presence of ethidium bromide.

### Example 4

### False Negative Error Rates were Estimated for the v1 Array

To further analyze results of array v1 tests as described in Example 2, false negative error rates were estimated for the v1 array. False negative error rates were estimated for experiments in which some or all of the viruses in the sample had known genome sequences (Table 7), and for probes that met Applicants' design criteria (85% identity and a 29 nt perfect match to one of the target genome sequences). The RSV and BTV probes were excluded from this estimate, as sequences were not available for the exact strains used in the experiments. All 128 selected probes had signals above the 99^{th} percentile detection threshold, yielding a zero false negative error rate.

TABLE 7
True positive/false negative counts for probes in MDA v1
tests with sequenced viruses.

| | Number | | | |
|---|---|---|---|---|
| | of PM | TP | FN | Percent FN |
| Target | probes | probes | probes | error rate |
| Pure viral cultures: | | | | |
| Adenovirus type 7 Gomen | 52 | 52 | 0 | 0.0 |
| Bovine viral diarrhea virus | 25 | 25 | 0 | 0.0 |
| (BVDV) | | | | |
| Mixture of viral cultures: | | | | |
| Human herpesvirus 6B | 14 | 14 | 0 | 0.0 |
| Vaccinia virus Lister strain | 37 | 37 | 0 | 0.0 |
| Total | 51 | 51 | 0 | 0.0% |
| Overall | 128 | 128 | 0 | 0.0% |

### Example 5

### Validation of Array v2 with Known Spiked Viruses

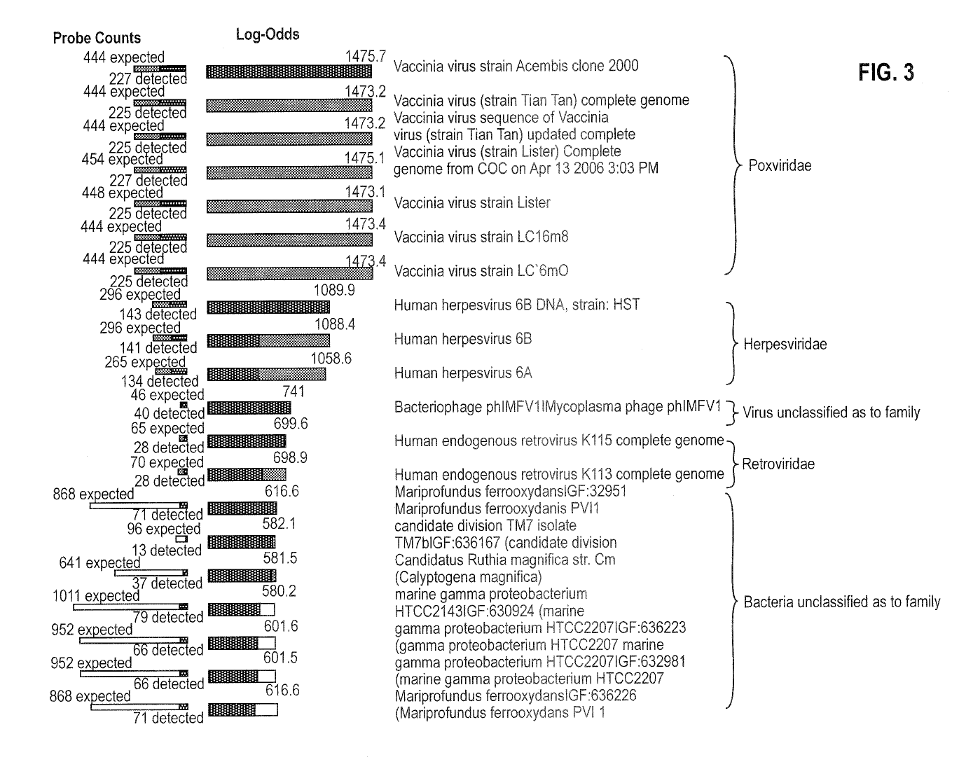

To validate v2 of the array with known spiked viruses, BVD type 1 (FIG. 2) and a mixture of vaccinia Lister and HHV 6B (FIG. 3) were tested on array v2. These organisms were correctly identified to the species level. Virus sequences selected as likely to be present are highlighted in red in these figures. On the vaccinia+HHV 6B array, human endogenous retrovirus K113 was also detected.

In addition, several organisms that were unlikely to be present were predicted, probably because of non-specific probe binding or cross-hybridization. These organisms, Mariprofundus ferrooxydans (a deep sea bacterium collected near Hawaii), candidate division TM7 (collected from a subgingival plaque in the human mouth), and marine gamma-proteobacterium (collected in the coastal Pacific Ocean at 10 m depth) were detected with low log-odds scores on numerous experiments using different samples. Genome sequences for these were not included in the probe design because they became available only after Applicants designed the microarray probes or because they were not classified into a bacterial taxonomic family; therefore probes were not screened for cross-hybridization against these targets. Genome comparisons indicate that M. ferrooxydans, TM7b, and marine gamma proteobacterium HTCC2143 share 70%, 55%, and 61%, respectively, of their sequence with other bacteria and viruses, based on simply considering every oligo of size at least 18 nt is also present in other sequenced viruses or bacteria, so many of the probes designed for other organisms may also hybridize to these targets.

### Example 6

### Testing on Blinded Samples from Pure Culture

To further test array v2, blinded samples from pure culture were tested. Blinded samples were provided from University of Texas, Medical Branch (UTMB) for 11 viruses. Applicants hybridized each of those samples separately to the MDA and predicted the identities of each virus (Table 8). 10 of 11 blinded samples were confirmed to be correctly identified by the MDA v2. VSV NJ was not detected in the 11th sample using the MDA, but was confirmed to be present by TaqMan PCR.

TABLE 8
Testing of array v2 on blinded samples from pure culture

| ID | Culture results | Array results |
|---|---|---|
| — | Vero Cells not infected | Background signal |
| TVP-11180 | Punta Toro | Punta Toro virus strain |
| | | Adames |
| TVP-11181 | Thogoto | Thogoto virus strain IIA |
| TVP-11182 | Dengue 4 | Dengue 4 strain |
| | | ThD4_0734_00 |
| TVP-11183 | CTF | Colorado tick fever virus |
| TVP-11184 | Cache Valley | Cache Valley genomic RNA |
| | | for N and NSs proteins |
| TVP-11185 | IIheus | IIheus virus |
| TVP-11186 | EHD-NJ | Epizootic hemorrhagic |
| | | disease virus isolate |
| | | 1999_MS-B NS3 |
| TVP-11187 | La Cross | La Crosse virus strain LACV |
| TVP-11188 | SF Sicilian | Sandfly fever sicilian virus |
| TVP-11189 | VSV-NJ | Not detected |
| TVP-11191 | Ross River | Ross River virus |

Ten of 11 of the species predicted by the MDA were confirmed. In addition, endogenous retroviruses were also detected by array v2 in 7 of the samples as well as the uninfected Vero cell control, indicating the presence of host DNA from the culture cells. These included one or more of the following: Baboon endogenous virus strain M7 and Human endogenous retroviruses K113, K115, and HCML-ARV, with Human endogenous retrovirus K113 being the most common.

The one sample that was not detected on the array was vesicular stomatitis virus, NJ (VSV NJ). VSV NJ was confirmed to be present in the sample using two proprietary, unpublished TaqMan assays developed by colleagues at LLNL and tested by LLNL colleagues at Plum Island that specifically detect VSV NJ. VSV NJ is a member of the Rhabdoviridae family, for which no genomes were available. Consequently, no probes were designed for this species and it was not represented in any database for the statistical analyses. It is sufficiently different from the genomes available for VSV Indiana that none of those probes had BLAST similarity to the partial sequences available for VSV NJ. There were 7 probes from the Virochip corresponding to VSV NJ that were detected. These probes were designed from partial sequences (see reference 23).

### Example 7

### Detection of Viruses and Bacteria from Clinical Samples with Array v1

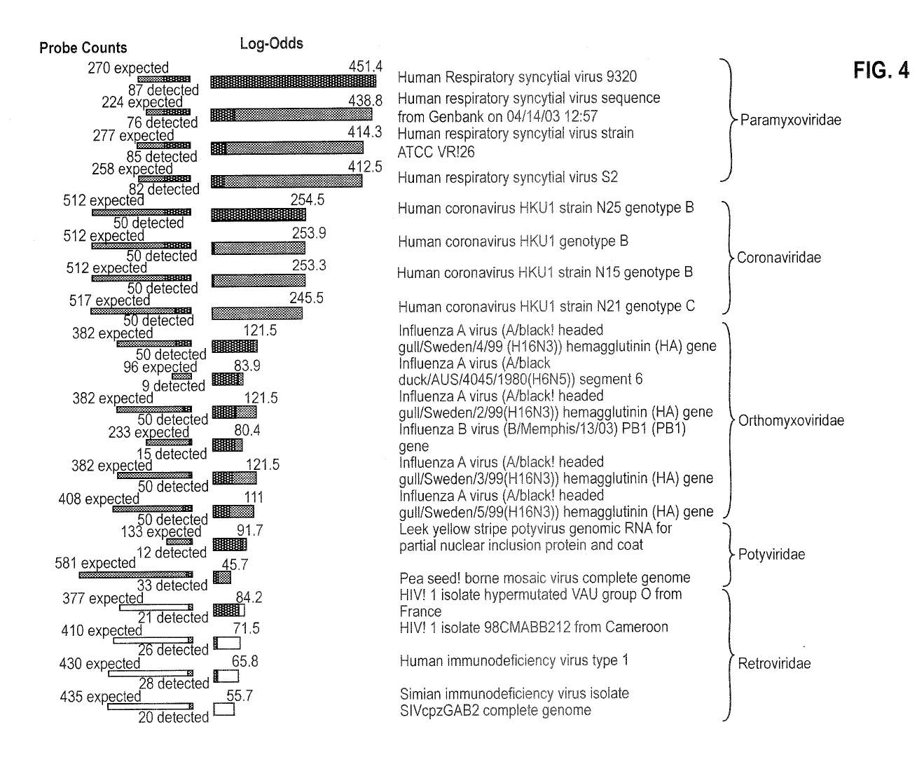

A clinical sputum sample provided from the UCSF DeRisi lab was tested on the MDA v1 (FIG. 4). Human respiratory syncytial virus and human coronavirus HKU1 were detected in this analysis. The length of a bar (FIG. 4) represents the log-likelihood contribution from probes with BLAST hits to the indicated sequence. The darker colored part of the bar represents the increase in log-likelihood that would result from adding the indicated target to the predicted set, not including contributions from previously predicted targets. Results were confirmed using specific PCR for these two viruses (Table 9). The results were also confirmed by the DeRisi lab using the ViroChip. The MDA results indicated small log-odds scores for influenza A, leek yellow stripe potyvirus, and HIV-1, although these low scores are a result of just a few probes and are likely due to nonspecific binding rather than true positives. Other samples tested using the MDA v1 also had a low likelihood predicted for Influenza A and Leek yellow stripe potyvirus (Table 6), and this is suspected to be due to non-specific binding, as discussed further in Example 8.

TABLE 9
Results from clinical samples - primer sequences, expected product sizes,
and results

| | | | | | Expected | |
|---|---|---|---|---|---|---|
| | SEQ | | SEQ | | Product | |
| | ID | Forward | ID | | Size | EPS |
| Sample | NO. | Primer | NO. | Reverse Primer | (EPS) | Detected |
| DeRset1_1 | | | | | | |
| Coronavirus | 133, | CTATGAA | 133, | GAACGGAACA | 287 | Yes |
| HKU1 | 264 | GTCAGAT | 265 | AGCCCATAAC | | |
| | | GAGGGTG | | ATA | | |
| | | GG | | | | |
| RSV | 133, | GGCAAAT | 133, | GACTCGTAGT | 224 | Yes |
| | 2663 | ATGGAAA | 267 | GAAGGTCCTT | | |
| | | CATACGTG | | TGG | | |
| | | AA | | | | |
| DeRsetDR210 | | | | | | |
| Human | 133, | AGATACC | 133, | GGGTTTGTTA | 180 | Yes |
| parechovirus 1 | 268 | ACGCTTGT | 269 | AACCTTGGCTT | | |
| isolate BNI-788St | | GGACCTTA | | TT | | |
| Streptococcus | 133, | CGTATCTG | 133, | CGCCCCAAAC | 265 | Yes |
| thermophilus | 270 | CCCGTATG | 271 | AAAGAATAGC | | |
| LMD9 | | CTTG | | | | |
| DeRsetDR220 | | | | | | |
| Escherichia coli | 133, | ATCCGTCA | 133, | AGAGAAAACG | 144 | Yes |
| CFT073 | 272 | TACGGAA | 273 | GAAGAGTATC | | |
| | | CATCAACT | | GCC | | |
| Norwalk virus 1 | 133, | GCTCCCAG | 133, | CACCATCATT | 60 | Yes |
| | 274 | TTTTGTGA | 275 | AGATGGAGCG | | |
| | | ATGAAGA | | G | | |
| Norwalk virus 2 | 133, | TTCACAAA | 133, | ATGGACTTTTA | 105 | Yes |
| | 276 | ACTGGGA | 277 | CGTGCC | | |
| | | GCC | | | | |
| DeRsetDR230 | | | | | | |
| Chicken anemia | 133, | GTTCAGGC | 133, | TTAGCTCGCTT | 258 | Yes |
| virus | 278 | CACCAAC | 279 | ACCCTGTACTC | | |
| | | AAGTTC | | G | | |
| Serratia | 133, | CCGCAGA | 133, | GCCGAATCAA | 203 | No |
| proteamaculans 1 | 280 | TCCTGGCT | 281 | CGAAGCCTAC | | |
| | | AAAA | | | | |
| Serratia | 133, | CCCTGGGT | 133, | CCCATAGCAC | 221 | No |
| proteamaculans 2 | 282 | AAGGTGA | 283 | CGCTTATCCT | | |
| | | AAACG | | | | |
| DeRsetDR240 | | | | | | |
| Staphylococcus | 133, | CATGCGTA | 133, | ATGCAAACGA | 281 | Yes |
| aureus | 284 | TTGCTATT | 285 | GTCCAAGCAG | | |
| | | GAGTTGC | | | | |
| Shigella & E. coli | 133, | CGTCTGCT | 133, | TCTCTTCTTCC | 239 | Yes |
| conserved region | 286 | GGATGGC | 287 | GGCACCATT | | |
| | | TTCTA | | | | |
| Shigella sonnei | 133, | GGGTGGA | 133, | GGCTCTGGAG | 287 | Yes |
| Ss046 plasmid | 288 | AAAGTTG | 289 | CAGGAAAAGA | | |
| pSS046_spB | | GGATCA | | | | |
| Lactococcus | 133, | AGGTGAC | 133, | TTCGCTTGTGT | 276 | Yes |
| lactis pGdh442 | 290 | CGTACTTT | 291 | TCGTCCTTG | | |
| plasmid | | ACACAAT | | | | |
| | | GG | | | | |
| Streptococcus | 133, | AACGAGC | 133, | TATGTACGGC | 300 | Yes |
| sanguinis | 292 | TGTTGAGG | 293 | GTCAAGGAGC | | |
| | | GCAAT | | | | |
| Lactococcus | 133, | TGGAAAA | 133, | TCGAGGGAAC | 232 | Yes |
| lactis pCI305 | 294 | TTGCGTCC | 295 | TGGGAATTTG | | |
| plasmid | | TTATTTG | | | | |
| E. coli pAPEC | 133, | CGGACGG | 133, | ATGCCTGCTC | 255 | No |
| O2-ColV plasmid | 296 | CTACTGAA | 297 | AACTCCATCA | | |
| 1 | | CCAAT | | | | |
| E. coli pAPEC | 133, | GCAGAAA | 133, | CTGAAGGCCA | 82 | No |
| O2-ColV plasmid | 298 | TGAAGCT | 299 | TCACCCGT | | |
| 2 | | GATGCG | | | | |

### Example 8

### Detection of Viruses and Bacteria from Clinical Samples with Array v2

Closer examination of probes giving high signal intensities that were not consistent with the “detected” organisms indicated the likelihood of some probes that bind non-specifically. On the MDA v2 array, 141 probes were detected in a majority (31 out of 60) of arrays hybridized to a wide variety of sample types. A small number of these probes were found to have significant BLAST hits to the human genome. Since most of the samples tested on the array were either human clinical samples or were grown in Vero cells (an African green monkey cell line), the frequent high signals for these few probes can be explained by the presence of primate DNA in the sample. The vast majority of spuriously binding probes, however, were not explained by cross-hybridization to host DNA. There were significant differences between non-specific and specific probes in the distributions of trimer entropy and hybridization free energy; non-specific probes had smaller entropies (mean 4.6 vs 4.8 bits, p=7.5×10⁻¹⁴) and more negative free energies (mean −70.5 vs −66.8 kcal/mol, p=3.8×10⁻¹³) compared to 1755 non-specific probes detected in 11 or fewer samples. Consequently, in v2 of the chip design, an entropy filter was imposed as described in the detailed description, and more probe sequences were designed at the expense of the number of replicates per probe.

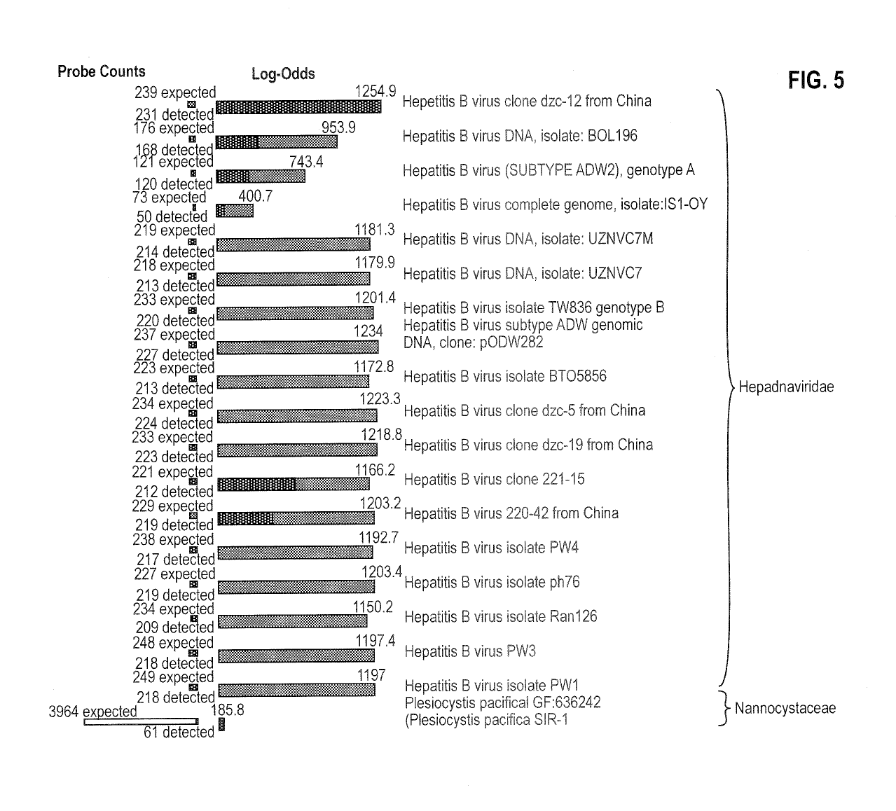

Partially amplified clinical samples provided by the DeRisi laboratory at UCSF were tested on the MDA v2. The source (e.g. fecal or serum) was blinded during experimentation and analysis, but was provided later. No patient history was provided. The results are shown in FIGS. 5-9.

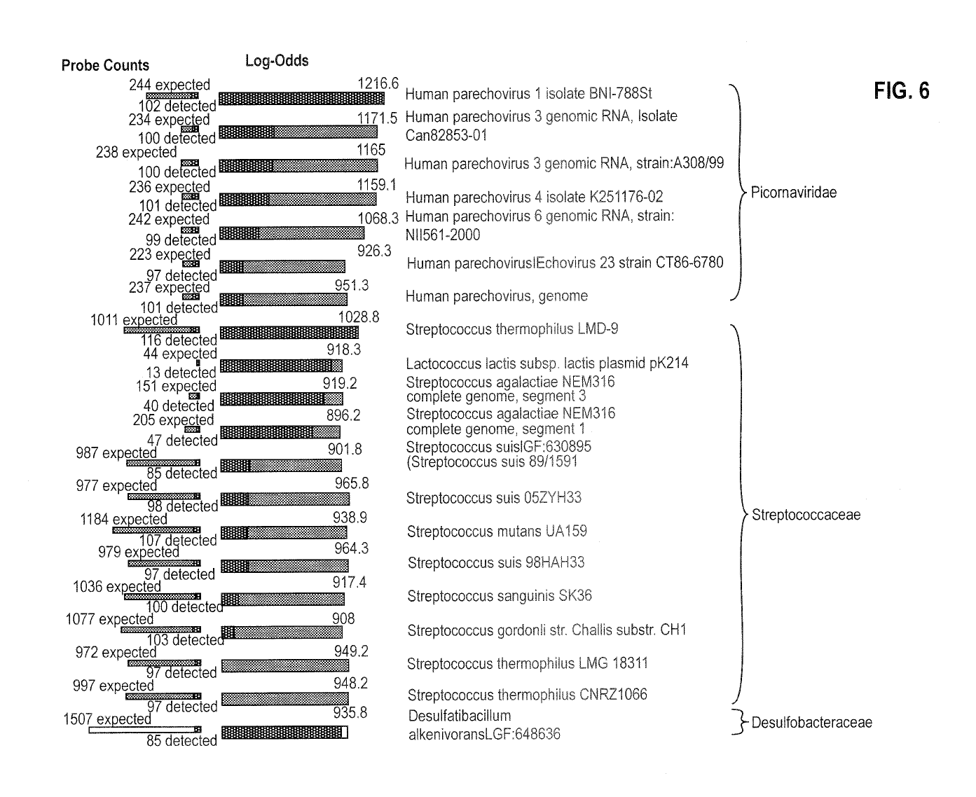

Hepatitis B virus was the only organism detected in sample 1_{—}5 (FIG. 5), and it produced a very strong signal. This was the only sample from a serum source. All the remaining samples (DR210, DR220, DR230, DR240) were from fecal sources. MDA v2 indicated that sample DR210 contained human parechovirus and a bacterium similar to Streptococcus thermophilus with a plasmid similar to one that has been sequenced from Lactococcus lactis (FIG. 6).

Other species of Streptococcaceae also had high log-odds ratios, consequently MDA v2 did not make a definitive call to the level of species. Streptococcus thermophilus is a gram-positive facultative anaerobe used as a fermenter for production of yogurt and mozzarella. It is also used as a probiotic to alleviate symptoms of lactose intolerance and gastrointestinal disturbances (see reference 12). Human parechoviruses cause mild gastrointestinal and respiratory illnesses. The presence of human parechovirus and Streptococcus thermophilus were confirmed by PCR (Table 9).

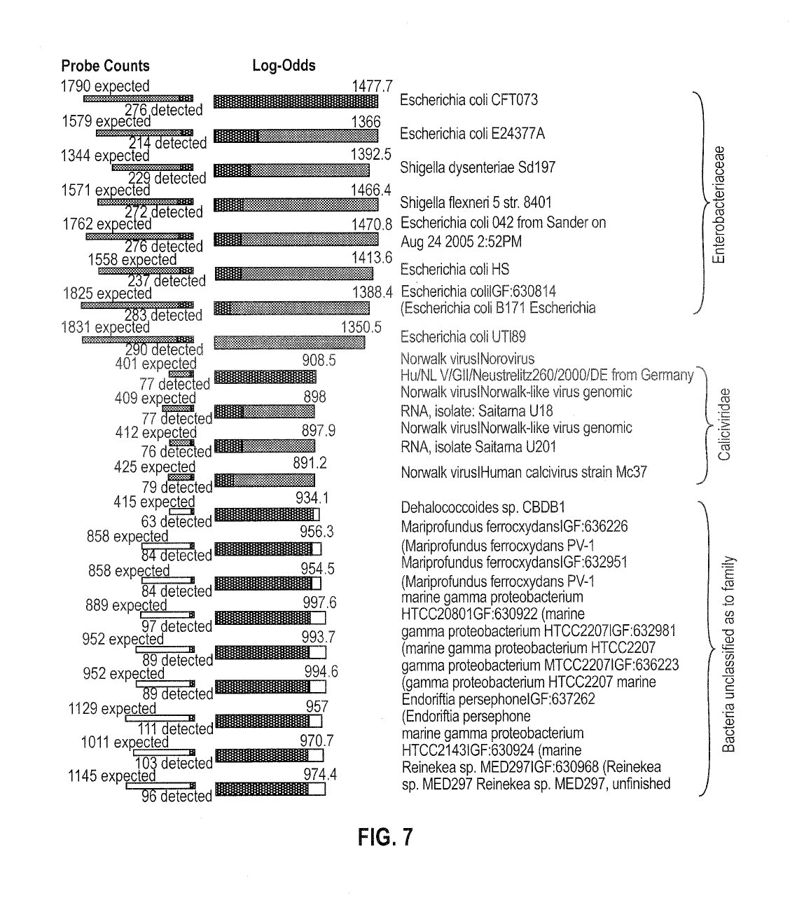

In sample DR220, Eschirichia coli CFT073 (or similar) and a Norwalk virus (FIG. 7) were identified. E. coli strain CFT073 is uropathogenic and is one of the most common causes of non-hospital acquired urinary tract infections, and Norwalk virus causes gastroenteritis. Since the probes were selected from conserved regions within a family, the array was not designed for stringent species or strain discrimination. A number of E. coli and Shigella genomes had nearly as high log-odds scores as E. coli CFT073. PCR confirmation was obtained for both E. coli and Norwalk virus (Table 9).

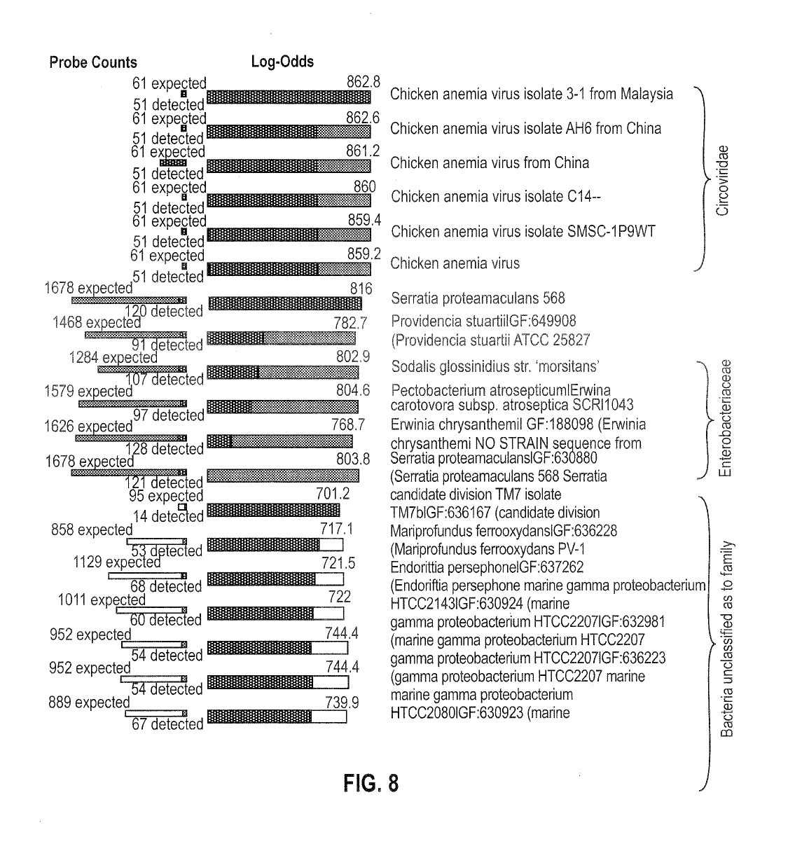

Sample DR230 was predicted to contain chicken anemia virus and Serratia proteamaculans or a related Enterobacteriaceae. S. proteamaculans has been associated with a severe form of pneumonia (see reference 2) (FIG. 8). The presence of chicken anemia was confirmed by PCR, but the presence of S. proteamaculans could not be confirmed.

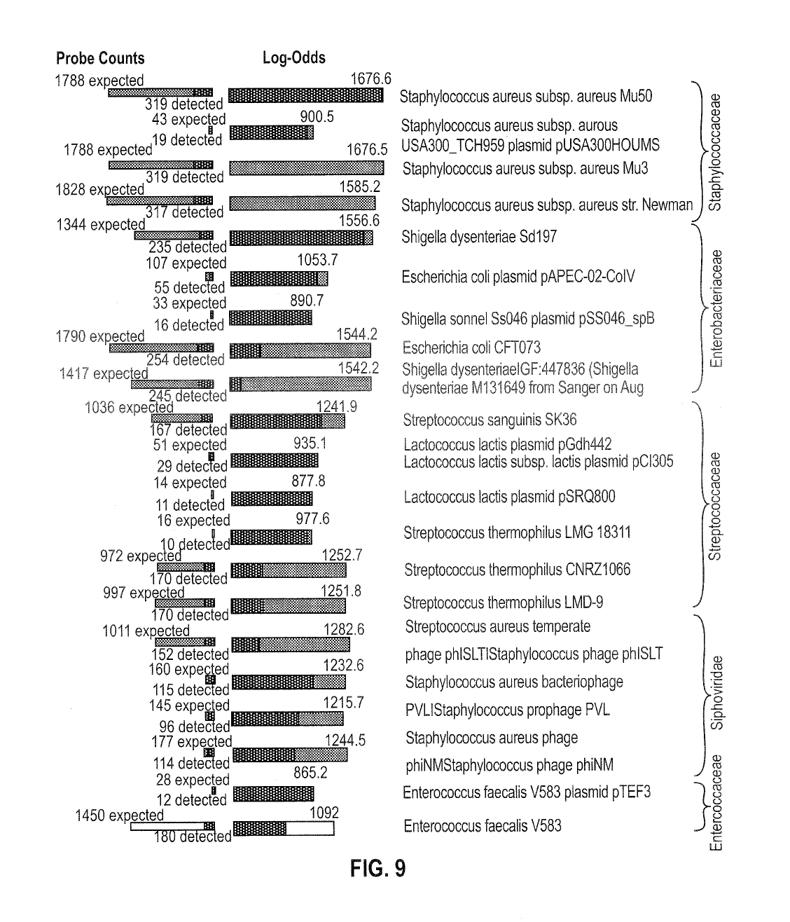

In sample DR240 only bacterial organisms were identified (FIG. 9). In particular, Staphylococcus aureus and an associated plasmid, Shigella dysentariae/E. coli and Shigella and E. coli plasmids, and Streptococcus sanguinis and related Lactococcus lactis plasmids were detected. All of these were confirmed by PCR except the E. coli pAPEC plasmid (Table 9).

### Example 9

### Limits of Detection and Hybridization Time for 4-Plex Array v2.1

Experiments were performed with the MDA v2.1 4-plex array to determine the minimum detectable quantity of viral DNA using the standard 17 hour hybridization time. In addition, experiments were conducted to determine whether shorter hybridization times could be used if there were a sufficient quantity or concentration of sample.

To test this, DNA was extracted from adenovirus type 7, Gomen strain. Sample DNA quantities ranging from 0.5 ng to 2000 ng were tested with 17 hour hybridizations, and amounts from 15.6 ng to 2000 ng were tested with 1 hour hybridizations. Arrays were analyzed with our standard maximum likelihood protocol. At 17 hours, the correct adenovirus strain was the top-scoring target for all but the smallest sample quantity tested; that is, DNA amounts as low as 1 ng (5×10⁷ genome copies) could be detected without sample amplification. With 1 hour hybridizations, the correct virus strain was identified at every DNA quantity tested, as low as 15.6 ng.

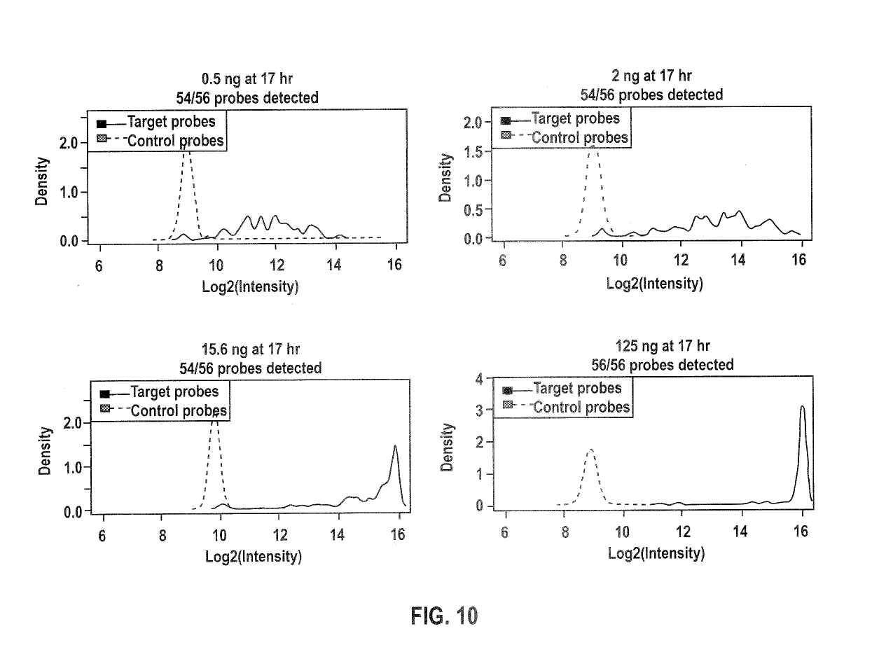

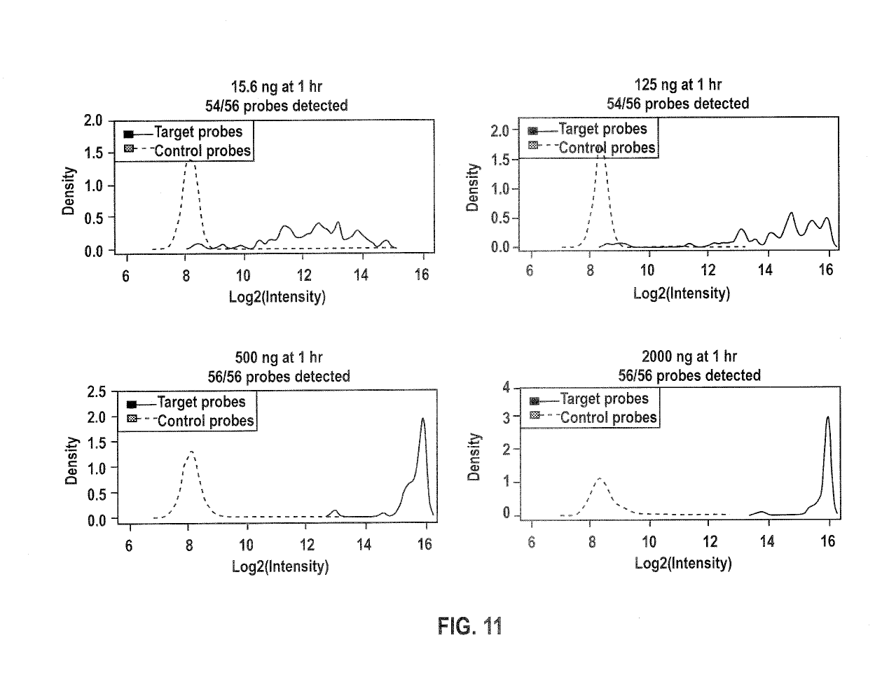

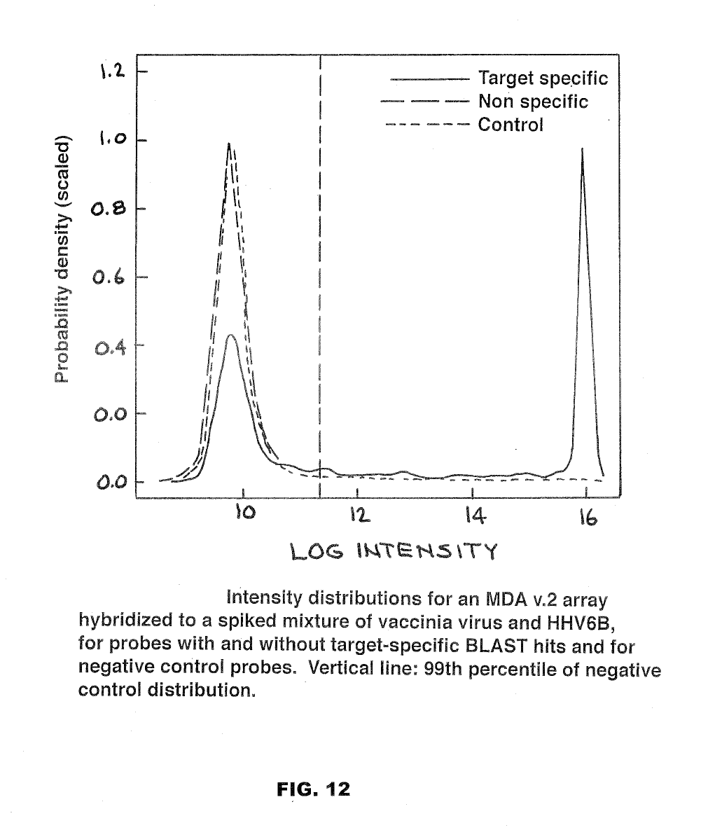

FIG. 10 shows the distribution of target-specific and negative control probe intensities observed in 4 of the 13 arrays hybridized for 17 hours at selected DNA concentrations; FIG. 11 displays corresponding distributions for 4 of the 8 one hour hybridizations at selected DNA concentrations. Separate density curves are shown for the negative control probes and the probes predicted to hybridize to the target virus genome, with detection probabilities greater than 95%. The target probes are clearly distinguished from the control probes in all cases. The target probe intensity distribution with 2 ng of DNA at 17 hours is similar to that observed with 15.6 ng at 1 hour. These results show that very short hybridization times can be used successfully when a sufficient amount of sample DNA is available.

### Example 10

### 135 Thousand Viral and Bacterial Probes for Clinical Microbial Detection Array

A detection microarray for targeting clinically relevant pathogens in a cost effective format (12×135K Nimblegen format) according to embodiments of the present disclosure is now described. The following example describes the design of a microarray for detecting vertebrate-infecting viruses and bacteria. The array includes 135 thousand probes from families known to infect vertebrates.

Complete viral and bacterial genome/segment/plasmid sequences were gathered from publicly available sites (Genbank, JCVI, IMG, etc.) and from collaborators (CDC), and were organized by family. Regions that were specific to a family were identified in which there were no regions longer than 17-23 bases that matched bacterial/viral genomes not in the target family or the human genome.

From these family-unique regions, candidate probes were identified to meet desired ranges for length (50-65 bases), Tm, entropy, GC %, and other thermodynamic and sequence features to the extent possible given the unique sequence. Detailed thermodynamic parameters are described in reference 28. The desired parameter ranges were relaxed as needed when there were too few probes for a target sequence, as Applicant's aimed at having between 5-40 probes per target (15 for most bacteria, 40 for most viruses), although there was variation around these numbers due to differences in target length and uniqueness.

Candidate probes were clustered and ranked within each family by the number of targets detected, and a greedy algorithm, as described was used to select a probe set to detect as many of the targets as possible with the fewest probes.

Uniqueness was calculated relative to all bacterial and viral families. However, only the probes for the clinically relevant families known to infect vertebrate hosts were included on the 135K clinical array. The viral families were selected from lists compiled by the International Committee on Taxonomy of Viruses and are available from virology.net/Big_Virology/BVHostList.html#Vertebrates

The following 33 viral families were included:

Adenoviridae, Alloherpesviridae, Anelloviridae, Arenaviridae, Arteriviridae, A sfarviridae, Astroviridae, Birnaviridae, Bornaviridae, Bunyaviridae, Caliciviridae, Circoviridae, Coronaviridae, Flaviviridae, Filoviridae, Hepeviridae, Hepadnaviridae, Herpesviridae, Iridoviridae, Nodaviridae, Orthomyxoviridae, Papillomaviridae, Paramyxoviridae, Parvoviridae, Picobirnaviridae, Picornaviridae, Polyomaviridae, Poxyiridae, Reoviridae, Retroviridae, Rhabdoviridae, Roniviridae, Togaviridae as well as one additional group, which is a genus, but has no family classification: Deltavirus.

The following bacterial families were included and were determined from extensive literature (PubMed) searches to determine if members of a family have been known to infect vertebrates or involved in clinical infections: Acetobacteraceae, Acholeplasmataceae, Actinomycetaceae, Actinosynnemataceae, Aerococcaceae, Aeromonadaceae, Alcaligenaceae, Anaeroplasmataceae, Anaplasmataceae, Bacillaceae, Bacteroidaceae, Bartonellaceae, Bdellovibrionaceae, Bifidobacteriaceae, Brachyspiraceae, Bradyrhizobiaceae, Brevibacteriaceae, Brucellaceae, Burkholderiaceae, Campylobacteraceae, Cardiobacteriaceae, Carnobacteriaceae, Catabacteriaceae, Caulobacteraceae, Cellulomonadaceae, Chlamydiaceae, Clostridiaceae, Clostridiales Family XI. Incertae Sedis, Clostridiales Family XI, Clostridiales Family XII. Incertae Sedis, Clostridiales Family XIII Incertae Sedis, Clostridiales Family XIV. Incertae Sedis, Clostridiales Family XV. Incertae Sedis, Clostridiales Family XVI. Incertae Sedis, Clostridiales Family XVIII. Incertae Sedis, Comamonadaceae, Coriobacteriaceae, Corynebacteriaceae, Coxiellaceae, Criblamydiaceae, Dermabacteraceae, Dermatophilaceae, Enterobacteriaceae, Enterococcaceae, Eubacteriaceae, Family X. Incertae Sedis, Family XVII. Incertae Sedis, Francisellaceae, Fusobacteriaceae, Gordoniaceae, Halomonadaceae, Helicobacteraceae, Jonesiaceae, Lachnospiraceae, Lactobacillaceae, Legionellaceae, Leptospiraceae, Leuconostocaceae, Listeriaceae, Methylobacteriaceae, Micrococcaceae, Moraxellaceae, Mycobacteriaceae, Mycoplasmataceae, Neisseriaceae, Nocardiaceae, Oxalobacteraceae, Parachlamydiaceae, Pasteurellaceae, Peptococcaceae, Peptostreptococcaceae, Piscirickettsiaceae, Pseudomonadaceae, Rickettsiaceae, Staphylococcaceae, Streptococcaceae, Vibrionaceae, Spirochaetaceae, Porphyromonadaceae, Prevotellaceae, Propionibacteriaceae, Rikenellaceae, Ruminococcaceae, Segniliparaceae, Simkaniaceae, Spirillaceae, Spiroplasmataceae, Sporolactobacillaceae, Streptomycetaceae. Succinivibrionaceae, Synergistaceae, Veillonellaceae, Victivallaceae, and Waddliaceae.

### Example 11

### 15 Thousand Viral Probes for Clinical Microbial Detection Array

A detection microarray targeting clinically relevant pathogens in a cost effective format (12×135K Nimblegen format) was designed. A subset of the probes in MDA v2 were downselected for inclusion in a Clinical 135K array, selecting probes for families known to infect vertebrate hosts and an additional set of 15K probes were designed specifically for this array.

The following example describes a microarray for viral and bacterial detection of organisms from families known to infect vertebrates. Many of the probes are a subset of the MDAv2 probes for the vertebrate-infecting families. A set of 14,996 viral probes were designed for this array.

For this array, the following steps were performed:

1) A complete viral genome and segment sequences were downloaded from the KPATH database in February 2011. These viral genomes and segment sequences were the target sequences for probe design.

2) A current complete set of sequences of fungi, bacteria, and archae were downloaded from the KPATH database in February 2011 for eliminating non-unique viral regions with respect to fungal, bacterial, and archaeal sequences.

3) In March 2011, current ribosomal sequences from the rRNA SILVA database were downloaded, human genome version 19 sequences, and repeat regions from the RepBase version 16.01 database, for eliminating non-unique viral regions with respect to rRNA, human, and repetitive sequences.

4) Family specific sequences were determined within each viral family by: using Vmatch software (Stephan Kurtz: The Vmatch large scale sequence analysis software, http://www.vmatch.de) to eliminate non-unique regions from the sequences in each vertebrate-infecting viral family. Uniqueness was determined with respect to “non-target” sequences, that is, the sequences in steps 3) and 4) above, as well as relative to any virus not in the viral family under consideration. Any region of 19 bases or longer with a perfect match in any non-target sequence was eliminated from consideration as a probe.

5) From the family specific sequences, probes were designed to meet desired ranges for length, Tm, entropy, GC %, and other thermodynamic and sequence features to the extent possible, relaxing the desired ranges as needed to obtain at least 5 probes per sequence, given sufficient unique regions exist for a sequence as described in Gardner et al., 2010, incorporated herein by reference in its entirety.

6) Candidate probes were clustered and ranked by the number of targets detected, and a greedy algorithm was used to select a probe set to detect as many of the targets as possible with the fewest probes, aiming for all sequences with sufficient unique regions at least 50 bases long to be represented by 5 probes. Targets with too little family specific sequence could have fewer probes in the total set of 15K designed. The algorithm was used to rank and downselect a probe set from the pool of candidate probes and is further described in reference 28.

The following 33 viral families were included:

Adenoviridae, Alloherpesviridae, Anelloviridae, Arenaviridae, Arteriviridae, Asfarviridae, Astroviridae, Birnaviridae, Bornaviridae, Bunyaviridae, Caliciviridae, Circoviridae, Coronaviridae, Flaviviridae, Filoviridae, Hepeviridae, Hepadnaviridae, Herpesviridae, Iridoviridae, Nodaviridae, Orthomyxoviridae, Papillomaviridae, Paramyxoviridae, Parvoviridae, Picobirnaviridae, Picornaviridae, Polyomaviridae, Poxyiridae, Reoviridae, Retroviridae, Rhabdoviridae, Roniviridae, Togaviridae, and one additional group, which is a genus, but has no family classification: Deltavirus.

### Example 12

### An Array Design

An array design process is diagrammed in FIGS. 1A and 1B. In designing probes for the array, Applicants sought to balance the goals of conservation and uniqueness, prioritizing oligo sequences that were conserved, to the extent possible, within the family of the targeted organism, and unique relative to other families and kingdoms. The design process is detailed in Methods, and summarized here.

Applicants designed arrays with larger numbers of probes per sequence (50 or more for viruses, 15 or more for bacteria) than previous arrays having only 2-10 probes per target. The large number of probes per target was expected to improve sensitivity, an important consideration given possible amplification bias in the random PCR sample preparation protocol, which could result in nonamplification of genome regions targeted by some probes [25]. All bacteria and viruses with sequenced genomes available at the time Applicants began the MDA v.1 design (spring 2007) were represented: ˜38,000 virus sequences representing ˜2200 species, and ˜3500 bacterial sequences representing ˜900 species. Version 1 of the array had only viral probes. A second version of the array (MDA v.2) was designed using both viral and bacterial probes. Probes were selected to avoid sequences with high levels of similarity to human, bacterial and viral sequences not in the target family. Low levels of sequence similarity across families were allowed selectively, when the statistical model of probe hybridization used in our array analysis predicted a low likelihood of cross-hybridization.

Favoring more conserved probes within a family enabled Applicants to minimize the total number of probes needed to cover all existing genomes with a high probe density per target, enhancing the capability to identify the species of known organisms and to detect unsequenced or emerging organisms. Strain or subtype identification was not a goal of probe design for this array. Nevertheless, Applicants ability to combine information from multiple probes in our analysis made it possible to discriminate between strains of many organisms.

The array design also incorporated a set of 2,600 negative control probes. These probes had sequences that were randomly generated, but with length and GC content distributions chosen to match those of the target-specific probes.

### Example 13

### Modeling of Probe Target Hybridization

A novel statistical method was developed for detection array analysis, by modeling the likelihood of the observed probe intensities as a function of the combination of targets present in the sample, and performing greedy maximization to find a locally optimal set of targets; the details of the algorithm are shown in Methods. It incorporates a probabilistic model of probe-target hybridization based on probe-target similarity and probe sequence complexity, with parameters fitted to experimental data from samples with known genome sequences. To accurately determine the organism(s) responsible for a given array result, the pattern of both positive and negative probe signals is taken into account. The algorithm is designed to enable quantifiable predictions of likelihood for the presence of multiple organisms in a complex sample.

A key simplification used in this algorithm was to transform the probe intensities to binary signal values (“positive” or “negative”), representing whether or not the intensity exceeds an array-specific detection threshold. The threshold was typically calculated as the 99^{th} percentile of the intensities of the random control probes on the array. The outcome variables in the likelihood model are the positive signal probabilities for each probe, given the presence of a particular combination of targets in the sample. The resulting predictions are more robust in the presence of noisy data, since the outcome variable is a probability rather than the actual intensity. Discretizing the intensities also led to considerable savings of computation time and resources, which are significant for arrays containing hundreds of thousands of probes.

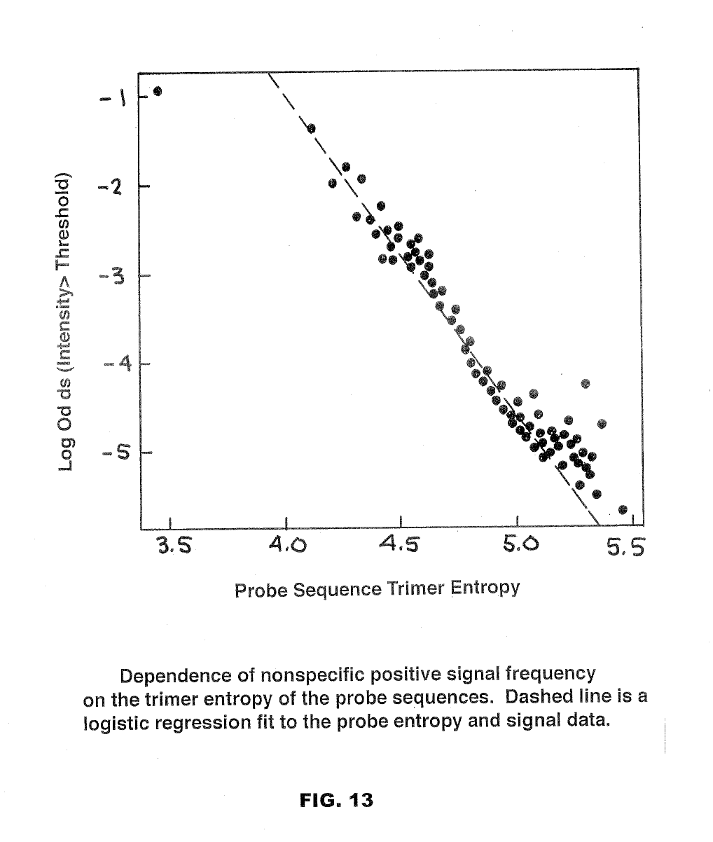

Although one might assume that reducing intensities to binary values means discarding valuable information, the log intensity distribution for a typical array (FIG. 13) shows that the actual information loss is much less than expected. FIG. 13 shows separate density curves for three classes of probes: those with BLAST hits to one of the known targets in the sample (“target-specific”), those without hits (“nonspecific”), and negative controls. A vertical dashed line is drawn at the 99^{th} percentile threshold intensity. Logₑ intensities for target-specific probes either cluster with the control and nonspecific probes (when they have low BLAST scores, usually), or approach the maximum possible value (16). This occurs because detection array probes are designed for high sensitivity to low target concentrations, so that probe intensities approach the saturation level whenever a probe has significant similarity to a target in the sample. Therefore, the information content of a probe signal is already reduced by saturation effects.

Certain probes were found to be more likely than others to yield positive signals, even when the sample on the array was known to lack any targets with sequences complementary to them. Applicants observed that this nonspecific hybridization occurs more often with probes having low sequence complexity, i.e. long homopolymers and tandem repeats. One measure of the complexity of a probe sequence is the entropy of its trimer frequency distribution.

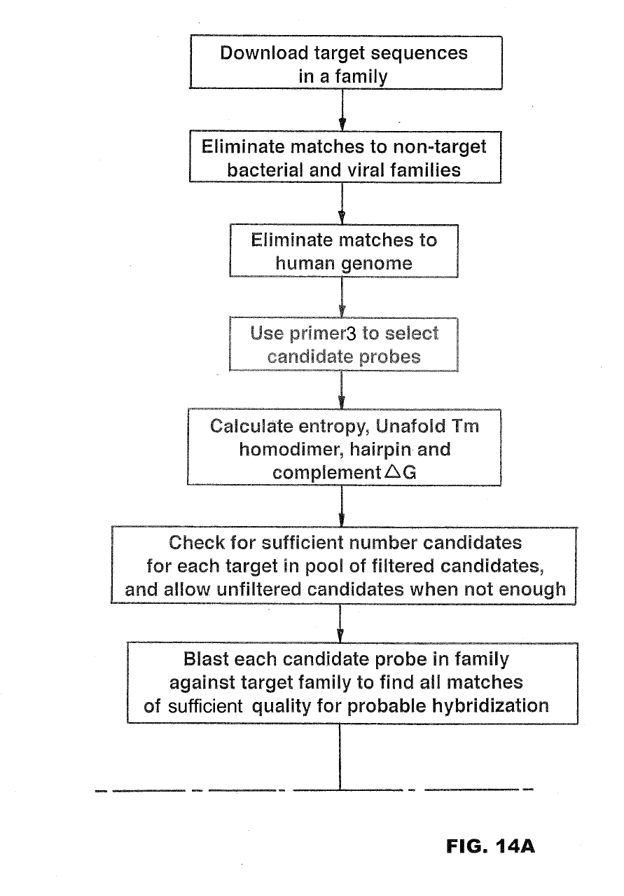

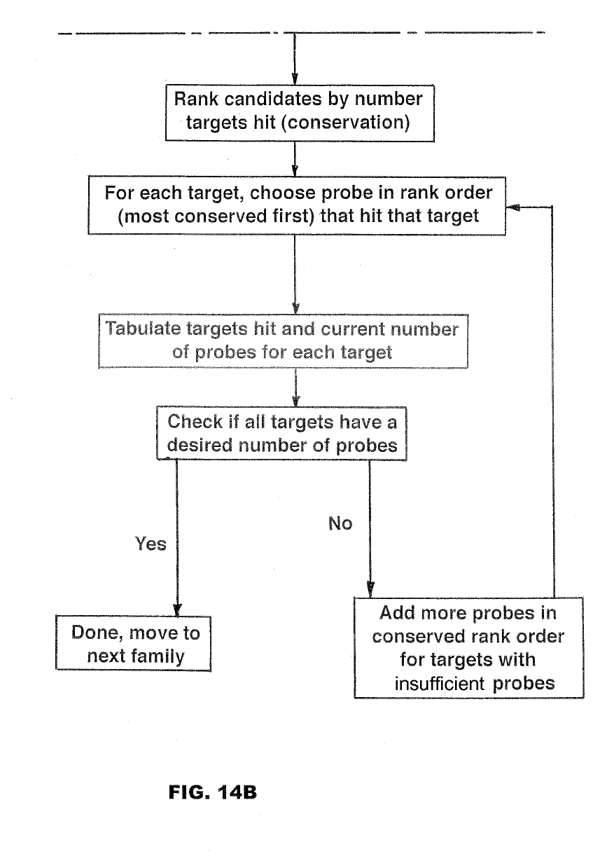

To study whether the sequence entropy could be used as a predictor of nonspecific hybridization, Applicants selected data from nine MDA v2 arrays for which all sample components had known genome sequences. Applicants selected probes with no BLAST hits to any of the known targets, grouped them by entropy into equal sized bins, computed the positive signal frequency (the fraction of probes with positive signals), converted the frequency to a log-odds value, and plotted the log-odds against the trimer entropy, as shown in FIGS. 14A and 14B. Applicants also fit a logistic regression model for the probe signal as a function of entropy; a dashed line with the resulting slope and intercept is shown in the plot. FIGS. 14A and 14B show that the trimer entropy is an excellent predictor of the non-specific positive signal probability, and that probes with low entropy are more likely to give positive signals regardless of the target sequence.

While the nonspecific probe signal probability depends on the probe sequence only, the target-specific signal probability was assumed to be a function of both the probe sequence and probe-target sequence similarity. To determine an appropriate set of predictors for the specific signal probability, given the presence of a specific target, Applicants BLASTed the probe sequences against our database of target genomes, obtaining the best alignment (if any) for each probe-target pair. Applicants then derived various covariates from the probe-target alignment, including the alignment length, number of mismatches, bit score, E-value, predicted melting temperature, and alignment start and end positions.

Applicants tested all combinations of up to three covariates, using logistic regression to fit models to data from samples containing known targets, and performed leave-one-out validation to find the combination with the strongest predictive value. The best combination included three covariates: (1) The predicted melting temperature, computed as described in Methods; (2) the BLAST bit score and (3) the alignment start position relative to the 5′ end of the probe. Applicants expected the alignment start position to have a significant effect, because in previous work [8] that probe-target mismatches had a weaker effect on hybridization if the mismatch was closer to the 3′ end of the probe (nearer to the array surface).

### Example 14

### A Set of Highly Conserved Probes

Of the 135K viral and bacterial probes identified in Example 12, a set of highly conserved probes was selected. Most of the probes can detect more than one species because they are highly conserved and selected so as to hit the most targets with the fewest probes as possible. The scoring algorithm that includes a contribution of numerous probes enables species resolution, even if a single probe is not sufficient.

The species listed as matching a probe can have some mismatches, although it is not likely enough to prevent hybridization. The species are listed for each probe for which there was a match of at least 50 bp and 90% similarity. The set of highly conserved probes comprise probes 1-63 which can detect bacterial species, probes 64-361 which can detect viral species, and probes 362-445 which can detect flu species and shown below in tables 10-12.

TABLE 10
Bacterial, viral, and flu species which can be detected by probes
corresponding to SEQ. ID NO. 1-445.

| SEQ ID NO | Detectable Species |
|---|---|
| 1 | Salmonella enterica |
| 1 | Yersinia pestis |
| 2 | Acinetobacter baumannii |
| 2 | Acinetobacter calcoaceticus |
| 2 | Acinetobacter sp. ADP1 |
| 3 | Bacillus anthracis |
| 3 | Bacillus cereus |
| 3 | Bacillus thuringiensis |
| 4 | Escherichia fergusonii |
| 4 | Klebsiella pneumoniae |
| 4 | Salmonella enterica |
| 5 | Enterococcus durans |
| 5 | Enterococcus faecalis |
| 5 | Enterococcus faecium |
| 6 | Yersinia enterocolitica |
| 6 | Yersinia pestis |
| 6 | Yersinia pseudotuberculosis |
| 6 | synthetic construct |
| 7 | Listeria monocytogenes |
| 7 | Macrococcus caseolyticus |
| 7 | Plasmid pSBK203 |
| 7 | Staphylococcus aureus |
| 7 | Staphylococcus epidermidis |
| 7 | Staphylococcus simulans |
| 8 | Escherichia coli |
| 8 | Klebsiella pneumoniae |
| 8 | Salmonella enterica |
| 8 | Shigella boydii |
| 8 | Shigella dysenteriae |
| 8 | Shigella flexneri |
| 8 | Shigella sonnei |
| 9 | Azotobacter vinelandii |
| 9 | Pseudomonas aeruginosa |
| 9 | Pseudomonas alkylphenolia |
| 9 | Pseudomonas brassicacearum |
| 9 | Pseudomonas entomophila |
| 9 | Pseudomonas fluorescens |
| 9 | Pseudomonas mendocina |
| 9 | Pseudomonas putida |
| 9 | Pseudomonas savastanoi |
| 9 | Pseudomonas sp. QDA |
| 9 | Pseudomonas syringae |
| 10 | Chlamydia trachomatis |
| 10 | Plasmid pCHL1 |
| 11 | Acinetobacter baumannii |
| 11 | Aeromonas hydrophila |
| 11 | Enterobacter aerogenes |
| 11 | Enterobacter cloacae |
| 11 | Escherichia coli |
| 11 | Klebsiella pneumoniae |
| 11 | Plasmid R751 |
| 11 | Salmonella enterica |
| 11 | Serratia marcescens |
| 11 | Shigella boydii |
| 11 | Shigella sonnei |
| 11 | Vibrio cholerae |
| 12 | Burkholderia ambifaria |
| 12 | Burkholderia cenocepacia |
| 12 | Burkholderia gladioli |
| 12 | Burkholderia glumae |
| 12 | Burkholderia mallei |
| 12 | Burkholderia multivorans |
| 12 | Burkholderia phymatum |
| 12 | Burkholderia phytofirmans |
| 12 | Burkholderia pseudomallei |
| 12 | Burkholderia sp. 383 |
| 12 | Burkholderia thailandensis |
| 12 | Burkholderia vietnamiensis |
| 12 | Burkholderia xenovorans |
| 12 | Cupriavidus pinatubonensis |
| 12 | Ricinus communis |
| 13 | Enterococcus faecalis |
| 13 | Staphylococcus aureus |
| 13 | Staphylococcus cohnii |
| 13 | Staphylococcus epidermidis |
| 13 | Staphylococcus haemolyticus |
| 13 | Staphylococcus |
| | pseudintermedius |
| 13 | Staphylococcus saprophyticus |
| 13 | Staphylococcus sciuri |
| 13 | Staphylococcus simulans |
| 13 | Staphylococcus sp. 693-7 |
| 13 | Staphylococcus warneri |
| 13 | Stenotrophomonas maltophilia |
| 14 | Francisella novicida |
| 14 | Francisella philomiragia |
| 14 | Francisella sp. TX077308 |
| 14 | Francisella tularensis |
| 14 | synthetic construct |
| 15 | Staphylococcus aureus |
| 16 | Plasmid pE5 |
| 16 | Plasmid pIM13 |
| 16 | Plasmid pNE131 |
| 16 | Plasmid pT48 |
| 16 | Reporter vector pGUSA |
| 16 | Shuttle vector pMTL85151 |
| 16 | Staphylococcus aureus |
| 16 | Staphylococcus haemolyticus |
| 16 | Staphylococcus lentus |
| 17 | Expression vector mce3 |
| 17 | Mycobacterium africanum |
| 17 | Mycobacterium bovis |
| 17 | Mycobacterium canettii |
| 17 | Mycobacterium tuberculosis |
| 18 | Cronobacter turicensis |
| 18 | Dickeya dadantii |
| 18 | Edwardsiella tarda |
| 18 | Enterobacter aerogenes |
| 18 | Enterobacter cloacae |
| 18 | Erwinia billingiae |
| 18 | Escherichia coli |
| 18 | Klebsiella pneumoniae |
| 18 | Pantoea agglomerans |
| 18 | Pantoea sp. At-9b |
| 18 | Rahnella aquatilis |
| 18 | Rahnella sp. Y9602 |
| 18 | Salmonella enterica |
| 18 | Serratia proteamaculans |
| 18 | Yersinia enterocolitica |
| 18 | Yersinia pestis |
| 18 | synthetic construct |
| 19 | Listeria grayi |
| 19 | Listeria innocua |
| 19 | Listeria monocytogenes |
| 20 | Alkaliphilus metalliredigens |
| 20 | Alkaliphilus oremlandii |
| 20 | Anaerococcus prevotii |
| 20 | Candidatus Arthromitus sp. |
| | SFB-rat-Yit |
| 20 | Clostridium acetobutylicum |
| 20 | Clostridium beijerinckii |
| 20 | Clostridium botulinum |
| 20 | Clostridium kluyveri |
| 20 | Clostridium ljungdahlii |
| 20 | Clostridium novyi |
| 20 | Clostridium perfringens |
| 20 | Clostridium tetani |
| 20 | Desulfitobacterium hafniense |
| 20 | Desulfotomaculum |
| | acetoxidans |
| 20 | Desulfotomaculum ruminis |
| 20 | Eubacterium limosum |
| 20 | Finegoldia magna |
| 20 | Nephroselmis olivacea |
| 20 | Thermincola potens |
| 21 | Arsenophonus nasoniae |
| 21 | Candidatus Moranella endobia |
| 21 | Citrobacter koseri |
| 21 | Citrobacter rodentium |
| 21 | Cronobacter sakazakii |
| 21 | Cronobacter turicensis |
| 21 | Dickeya dadantii |
| 21 | Dickeya zeae |
| 21 | Edwardsiella ictaluri |
| 21 | Edwardsiella tarda |
| 21 | Enterobacter aerogenes |
| 21 | Enterobacter asburiae |
| 21 | Enterobacter cloacae |
| 21 | Enterobacter sp. 638 |
| 21 | Erwinia amylovora |
| 21 | Erwinia billingiae |
| 21 | Erwinia pyrifoliae |
| 21 | Erwinia sp. Ejp617 |
| 21 | Erwinia tasmaniensis |
| 21 | Escherichia coli |
| 21 | Escherichia fergusonii |
| 21 | Ferrimonas balearica |
| 21 | Klebsiella pneumoniae |
| 21 | Klebsiella variicola |
| 21 | Pantoea ananatis |
| 21 | Pantoea sp. At-9b |
| 21 | Pantoea vagans |
| 21 | Pectobacterium atrosepticum |
| 21 | Pectobacterium carotovorum |
| 21 | Pectobacterium wasabiae |
| 21 | Photorhabdus asymbiotica |
| 21 | Photorhabdus luminescens |
| 21 | Proteus mirabilis |
| 21 | Rahnella sp. Y9602 |
| 21 | Salmonella bongori |
| 21 | Salmonella enterica |
| 21 | Serratia marcescens |
| 21 | Serratia proteamaculans |
| 21 | Serratia sp. AS13 |
| 21 | Shigella boydii |
| 21 | Shigella dysenteriae |
| 21 | Shigella flexneri |
| 21 | Shigella sonnei |
| 21 | Sodalis glossinidius |
| 21 | Xenorhabdus bovienii |
| 21 | Xenorhabdus nematophila |
| 21 | Yersinia enterocolitica |
| 21 | Yersinia pestis |
| 21 | Yersinia pseudotuberculosis |
| 21 | synthetic construct |
| 22 | Neisseria gonorrhoeae |
| 22 | Neisseria lactamica |
| 22 | Neisseria meningitidis |
| 23 | Enterococcus faecalis |
| 23 | Enterococcus faecium |
| 23 | Enterococcus sp. 7L76 |
| 24 | Mariner transposase delivery |
| | vector pFA545 |
| 24 | Plasmid pNS1 |
| 24 | Plasmid pT181 |
| 24 | Single-copy integration vector |
| | pLL39 |
| 24 | Single-copy integtation vector |
| | pLL29 |
| 24 | Staphylococcus aureus |
| 24 | Staphylococcus epidermidis |
| 24 | Staphylococcus lentus |
| 25 | Bacteroides fragilis |
| 26 | Yersinia pestis |
| 27 | Yersinia enterocolitica |
| 28 | Enterococcus faecalis |
| 29 | Clostridium perfringens |
| 30 | Escherichia coli |
| 30 | Shigella sonnei |
| 30 | Yersinia pestis |
| 31 | Staphylococcus aureus |
| 31 | Staphylococcus carnosus |
| 31 | Staphylococcus epidermidis |
| 31 | Staphylococcus haemolyticus |
| 31 | Staphylococcus lugdunensis |
| 31 | Staphylococcus saprophyticus |
| 32 | Haemophilus ducreyi |
| 33 | Propionibacterium acnes |
| 34 | Burkholderia ambifaria |
| 34 | Burkholderia cenocepacia |
| 34 | Burkholderia gladioli |
| 34 | Burkholderia glumae |
| 34 | Burkholderia mallei |
| 34 | Burkholderia multivorans |
| 34 | Burkholderia pseudomallei |
| 34 | Burkholderia sp. 383 |
| 34 | Burkholderia thailandensis |
| 34 | Burkholderia vietnamiensis |
| 35 | Campylobacter jejuni |
| 35 | Campylobacter lari |
| 36 | Chlamydia muridarum |
| 36 | Chlamydia trachomatis |
| 36 | Chlamydophila abortus |
| 36 | Chlamydophila caviae |
| 36 | Chlamydophila felis |
| 36 | Chlamydophila pecorum |
| 36 | Chlamydophila pneumoniae |
| 36 | Chlamydophila psittaci |
| 37 | Coraliomargarita akajimensis |
| 37 | Orientia tsutsugamushi |
| 37 | Rickettsia africae |
| 37 | Rickettsia akari |
| 37 | Rickettsia bellii |
| 37 | Rickettsia canadensis |
| 37 | Rickettsia conorii |
| 37 | Rickettsia felis |
| 37 | Rickettsia heilongjiangensis |
| 37 | Rickettsia japonica |
| 37 | Rickettsia massiliae |
| 37 | Rickettsia peacockii |
| 37 | Rickettsia prowazekii |
| 37 | Rickettsia rickettsii |
| 37 | Rickettsia typhi |
| 38 | Cloning vector pKEK1140 |
| 38 | Francisella complementation |
| | plasmid pFNLTP23 |
| 38 | Francisella novicida |
| 38 | Francisella tularensis |
| 38 | Himar1-delivery and |
| | mutagenesis vector |
| | pFNLTP16 H3 |
| 38 | Shuttle vector pXB173-lux |
| 38 | Temperature-sensitive shuttle |
| | vector pFNLTP9 |
| 39 | Listonella anguillarum |
| 39 | Vibrio cholerae |
| 39 | Vibrio furnissii |
| 39 | Vibrio vulnificus |
| 39 | synthetic construct |
| 40 | Brucella abortus |
| 40 | Brucella canis |
| 40 | Brucella melitensis |
| 40 | Brucella microti |
| 40 | Brucella ovis |
| 40 | Brucella pinnipedialis |
| 40 | Brucella suis |
| 40 | Mesorhizobium ciceri |
| 40 | Mesorhizobium loti |
| 40 | Mesorhizobium opportunistum |
| 40 | Ochrobactrum anthropi |
| 41 | Escherichia coli |
| 41 | Klebsiella pneumoniae |
| 41 | Plasmid F |
| 41 | Plasmid R100 |
| 41 | Plasmid R65 |
| 41 | Salmonella enterica |
| 41 | Shigella boydii |
| 41 | Shigella dysenteriae |
| 41 | Shigella flexneri |
| 41 | Shigella sonnei |
| 41 | uncultured bacterium |
| 42 | Klebsiella pneumoniae |
| 42 | Kluyvera intermedia |
| 42 | Plasmid pYVe439-80 |
| 42 | Salmonella enterica |
| 42 | Yersinia enterocolitica |
| 42 | Yersinia pestis |
| 42 | Yersinia pseudotuberculosis |
| 43 | Escherichia coli |
| 43 | Plasmid ColE1 |
| 43 | Shigella boydii |
| 43 | Shigella sonnei |
| 43 | unidentified cloning vector |
| 44 | Campylobacter jejuni |
| 44 | Campylobacter lari |
| 45 | Brucella abortus |
| 45 | Brucella canis |
| 45 | Brucella melitensis |
| 45 | Brucella microti |
| 45 | Brucella ovis |
| 45 | Brucella pinnipedialis |
| 45 | Brucella suis |
| 45 | Ochrobactrum anthropi |
| 46 | Treponema pallidum |
| 46 | Treponema paraluiscuniculi |
| 47 | Clostridium botulinum |
| 48 | Streptococcus agalactiae |
| 48 | Streptococcus dysgalactiae |
| 48 | Streptococcus gallolyticus |
| 48 | Streptococcus gordonii |
| 48 | Streptococcus mitis |
| 48 | Streptococcus mutans |
| 48 | Streptococcus oralis |
| 48 | Streptococcus parauberis |
| 48 | Streptococcus pasteurianus |
| 48 | Streptococcus pneumoniae |
| 48 | Streptococcus |
| | pseudopneumoniae |
| 48 | Streptococcus pyogenes |
| 48 | Streptococcus salivarius |
| 48 | Streptococcus thermophilus |
| 48 | Streptococcus uberis |
| 48 | uncultured bacterium MID12 |
| 49 | Bursa aurealis delivery vector |
| | pBursa |
| 49 | Cloning vector pVLG6 |
| 49 | Expression vector pTSC |
| 49 | Plasmid pE194 |
| 49 | Shuttle vector pASD2 |
| 49 | Staphylococcus aureus |
| 49 | Tn10 delivery vector |
| | pHV1249 |
| 49 | synthetic construct |
| 50 | Chlamydia muridarum |
| 51 | Enterococcus caccae |
| 51 | Enterococcus casseliflavus |
| 51 | Enterococcus durans |
| 51 | Enterococcus faecalis |
| 51 | Enterococcus faecium |
| 51 | Enterococcus haemoperoxidus |
| 51 | Enterococcus hirae |
| 51 | Enterococcus moraviensis |
| 51 | Enterococcus mundtii |
| 51 | Enterococcus plantarum |
| 51 | Enterococcus quebecensis |
| 51 | Enterococcus ratti |
| 51 | Enterococcus silesiacus |
| 51 | Enterococcus sp. 7L76 |
| 51 | Enterococcus termitis |
| 51 | Enterococcus thailandicus |
| 51 | Enterococcus ureasiticus |
| 51 | Enterococcus villorum |
| 51 | Lactobacillus vaginalis |
| 52 | Escherichia coli |
| 52 | Klebsiella pneumoniae |
| 52 | Salmonella enterica |
| 52 | Shigella flexneri |
| 52 | Yersinia pestis |
| 53 | Citrobacter koseri |
| 53 | Enterobacter hormaechei |
| 53 | Escherichia coli |
| 53 | Klebsiella pneumoniae |
| 53 | Photorhabdus asymbiotica |
| 53 | Yersinia pestis |
| 54 | Enterococcus faecium |
| 54 | Macrococcus caseolyticus |
| 54 | Staphylococcus aureus |
| 54 | Staphylococcus epidermidis |
| 55 | Bacteroides fragilis |
| 55 | uncultured bacterium |
| 55 | uncultured organism |
| 56 | Staphylococcus aureus |
| 56 | Staphylococcus chromogenes |
| 56 | Staphylococcus epidermidis |
| 56 | Staphylococcus haemolyticus |
| 56 | Staphylococcus simulans |
| 56 | Staphylococcus sp. |
| 57 | Bacillus anthracis |
| 57 | Bacillus cereus |
| 57 | Bacillus thuringiensis |
| 57 | Bacillus weihenstephanensis |
| 57 | synthetic construct |
| 58 | Plasmid pKYM |
| 58 | Shigella boydii |
| 58 | Shigella sonnei |
| 59 | Listeria grayi |
| 59 | Listeria innocua |
| 59 | Listeria ivanovii |
| 59 | Listeria monocytogenes |
| 59 | Listeria seeligeri |
| 59 | Listeria welshimeri |
| 60 | Staphylococcus aureus |
| 60 | Staphylococcus epidermidis |
| 60 | Staphylococcus haemolyticus |
| 60 | Staphylococcus lugdunensis |
| 60 | Staphylococcus |
| | pseudintermedius |
| 60 | Staphylococcus simulans |
| 60 | Staphylococcus sp. CDC25 |
| 61 | Brucella abortus |
| 61 | Brucella canis |
| 61 | Brucella melitensis |
| 61 | Brucella microti |
| 61 | Brucella ovis |
| 61 | Brucella pinnipedialis |
| 61 | Brucella suis |
| 61 | Ochrobactrum anthropi |
| 62 | Enterococcus faecalis |
| 62 | Enterococcus faecium |
| 62 | Lactobacillus brevis |
| 62 | Lactobacillus fermentum |
| 62 | Lactobacillus plantarum |
| 62 | Lactobacillus rennini |
| 62 | Lactococcus lactis |
| 62 | Leuconostoc mesenteroides |
| 62 | Plasmid pCD4 |
| 62 | Shuttle vector pLES003 |
| 63 | Bacteroides fragilis |
| 63 | Bacteroides helcogenes |
| 63 | Bacteroides thetaiotaomicron |
| 63 | Bacteroides xylanisolvens |
| 64 | Lassa virus |
| 65 | Human papillomavirus type 148 |
| 66 | Camelpox virus |
| 66 | Cowpox virus |
| 66 | Ectromelia virus |
| 66 | Monkeypox virus |
| 66 | Taterapox virus |
| 66 | Vaccinia virus |
| 66 | Variola virus |
| 67 | Seoul virus |
| 68 | California sea lion astrovirus |
| | 11 |
| 68 | Human astrovirus |
| 69 | Guanarito virus |
| 70 | GB virus A |
| 71 | Human rotavirus B219 |
| 71 | Rotavirus B |
| 72 | Antwerp rhinovirus 98/99 |
| 72 | Chimpanzee enterovirus CPS- |
| | 2011 |
| 72 | Coxsackievirus |
| 72 | Enterovirus LaN/98/CH |
| 72 | Enterovirus sp. |
| 72 | Human echovirus AMS573 |
| 72 | Human enterovirus A |
| 72 | Human rhinovirus sp. |
| 72 | Porcine enterovirus B |
| 72 | Simian enterovirus SV19 |
| 72 | Simian picornavirus strain |
| | N125 |
| 72 | uncultured enterovirus |
| 73 | Machupo virus |
| 74 | Machupo virus |
| 75 | Rotavirus A |
| 75 | Rotavirus C |
| 75 | Rotavirus sp. |
| 76 | Human papillomavirus 109 |
| 77 | Rift Valley fever virus |
| 78 | Human herpesvirus 8 |
| 79 | Lassa virus |
| 80 | Human papillomavirus 50 |
| 81 | California encephalitis virus |
| 81 | Marituba virus |
| 82 | Hepatitis GB virus B |
| 82 | synthetic construct |
| 83 | Rift Valley fever virus |
| 84 | Chimeric Dengue virus vector |
| | p4(Delta30)-D2-CME |
| 84 | Chimeric Tick-borne |
| | encephalitis virus/Dengue |
| | virus 4 |
| 84 | Chimeric dengue virus type 1 |
| | vector p4(delta)30-D1L-CME |
| 84 | Dengue virus |
| 85 | Equine rotavirus |
| 85 | Rotavirus A |
| 85 | Rotavirus C |
| 85 | Rotavirus sp. |
| 86 | Rift Valley fever virus |
| 87 | Human papillomavirus 61 |
| 88 | Norwalk virus |
| 89 | Crane hepatitis B virus |
| 89 | Duck hepatitis B virus |
| 89 | Heron hepatitis B virus |
| 89 | Ross's goose hepatitis B virus |
| 89 | Sheldgoose hepatitis B virus |
| 90 | Rotavirus A |
| 91 | Human herpesvirus 4 |
| 92 | Human herpesvirus 2 |
| 93 | Murine norovirus |
| 93 | Norwalk virus |
| 94 | Bat coronavirus BM48- |
| | 31/BGR/2008 |
| 94 | Severe acute respiratory |
| | syndrome-related coronavirus |
| 94 | recombinant SARS |
| | coronavirus |
| 94 | recombinant coronavirus |
| 94 | synthetic construct |
| 95 | Eastern equine encephalitis |
| | virus |
| 96 | Amapari virus |
| 96 | Guanarito virus |
| 97 | Human respiratory syncytial |
| | virus |
| 97 | Respiratory syncytial virus |
| 98 | GB virus A |
| 99 | Feline rotavirus |
| 99 | Rotavirus A |
| 99 | Rotavirus C |
| 100 | AdEasy vector pShuttle |
| 100 | Adenoviral expression vector |
| | Ad-hiNOS |
| 100 | Adenoviral vector Ad-SAR1- |
| | x/ASX |
| 100 | Cloning vector |
| | pdeltaE1sp1A(CMV-GFP) |
| 100 | EGFP expression vector Ad- |
| | EGFP |
| 100 | Homo sapiens |
| 100 | Human adenovirus C |
| 100 | Recombination vector |
| | pAdHTS |
| 100 | Shuttle vector pSC- |
| | R1LambdaR2 |
| 100 | synthetic construct |
| 101 | Human herpesvirus 5 |
| 102 | Human papillomavirus 48 |
| 103 | Human herpesvirus 7 |
| 104 | Human papillomavirus 1 |
| 105 | Human papillomavirus 26 |
| 106 | Bovine enteric calicivirus |
| 106 | Caliciviridae |
| | bovine/DijonA058/05/FR |
| 106 | Caliciviridae |
| | bovine/DijonA386/08/FR |
| 106 | Calicivirus isolate TCG |
| 106 | Calicivirus strain CV23-OH |
| 106 | Newbury-1 virus |
| 107 | Human rotavirus ADRV-N |
| 107 | Rotavirus B |
| 108 | Human papillomavirus 92 |
| 109 | Human papillomavirus 32 |
| 110 | Human herpesvirus 3 |
| 111 | Hendra virus |
| 111 | Nipah virus |
| 112 | European brown hare |
| | syndrome virus |
| 113 | Bat picornavirus 3 |
| 113 | Chimpanzee enterovirus CPS- |
| | 2011 |
| 113 | EIAV-based lentiviral vector |
| 113 | Enterovirus sp. |
| 113 | Human echovirus AMS573 |
| 113 | Human enterovirus D |
| 113 | Human rhinovirus C |
| 113 | Porcine enterovirus B |
| 113 | Simian enterovirus SV19 |
| 113 | synthetic construct |
| 113 | uncultured enterovirus |
| 114 | Hantavirus Yakeshi-Mm-59 |
| 114 | Khabarovsk virus |
| 115 | California encephalitis virus |
| 116 | Rotavirus A |
| 117 | Measles virus |
| 118 | Lymphocytic choriomeningitis |
| | virus |
| 119 | Lassa virus |
| 120 | Kyasanur forest disease virus |
| 121 | Human papillomavirus 54 |
| 122 | Hepatitis C virus |
| 122 | synthetic construct |
| 123 | Human papillomavirus 63 |
| 124 | GB virus C |
| 125 | Hantaan virus |
| 126 | Human papillomavirus 60 |
| 127 | Human papillomavirus 16 |
| 128 | Crimean-Congo hemorrhagic |
| | fever virus |
| 129 | Rotavirus A |
| 130 | Rotavirus A |
| 131 | Reston ebolavirus |
| 132 | Human herpesvirus 6 |
| 133 | Norwalk virus |
| 134 | Homo sapiens |
| 134 | Human papillomavirus 18 |
| 135 | Sapporo virus |
| 136 | Rotavirus A |
| 136 | Rotavirus C |
| 137 | Human papillomavirus 7 |
| 138 | Hantavirus CGRn8316 |
| 138 | Hantavirus CGRn9415 |
| 138 | Seoul virus |
| 139 | Human papillomavirus type |
| | 128 |
| 140 | El Moro Canyon virus |
| 140 | Playa de Oro hantavirus |
| 140 | Prairie vole hantavirus |
| 140 | Rio Segundo virus |
| 141 | Rotavirus A |
| 141 | Rotavirus sp. |
| 142 | California encephalitis virus |
| 143 | Chikungunya virus |
| 143 | Cloning vector pCHIK-LR |
| | 5′GFP |
| 143 | O'nyong-nyong virus |
| 145 | Rotavirus A |
| 145 | Rotavirus sp. |
| 146 | Sapporo virus |
| 147 | Human papillomavirus 116 |
| 148 | Human papillomavirus 18 |
| 149 | Duck hepatitis A virus |
| 150 | Human papillomavirus 26 |
| 151 | Rotavirus A |
| 152 | St-Valerien swine virus |
| 153 | Rotavirus A |
| 154 | Human papillomavirus 2 |
| 155 | Human papillomavirus 34 |
| 156 | Rotavirus A |
| 156 | Rotavirus C |
| 157 | Zaire ebolavirus |
| 158 | Crimean-Congo hemorrhagic |
| | fever virus |
| 159 | Feline rotavirus |
| 159 | Rotavirus A |
| 160 | Rotavirus A |
| 161 | Lymphocytic choriomeningitis |
| | virus |
| 162 | Lake Victoria marburgvirus |
| 163 | Rotavirus A |
| 163 | Rotavirus sp. |
| 164 | Rotavirus A |
| 165 | Hepatitis A virus |
| 166 | Human papillomavirus 6 |
| 167 | Rotavirus A |
| 168 | Human papillomavirus 10 |
| 169 | Human papillomavirus 112 |
| 170 | Rotavirus A |
| 171 | Bagaza virus |
| 171 | Koutango virus |
| 171 | St. Louis encephalitis virus |
| 172 | Sapporo virus |
| 173 | Colobus monkey |
| | papillomavirus |
| 173 | Human papillomavirus 5 |
| 174 | Feline rotavirus |
| 174 | Rotavirus A |
| 174 | Rotavirus C |
| 175 | Human papillomavirus type |
| | 134 |
| 176 | Rotavirus A |
| 176 | Rotavirus sp. |
| 177 | Human papillomavirus 109 |
| 178 | Japanese encephalitis virus |
| 178 | Murray Valley encephalitis |
| | virus |
| 178 | Usutu virus |
| 178 | West Nile virus |
| 178 | synthetic construct |
| 179 | Mopeia Lassa reassortant 29 |
| 179 | Mopeia virus |
| 180 | Human papillomavirus 7 |
| 181 | Human papillomavirus 18 |
| 182 | Rotavirus A |
| 183 | Murine rotavirus |
| 183 | Rotavirus A |
| 183 | Rotavirus C |
| 184 | Norwalk virus |
| 185 | Crimean-Congo hemorrhagic |
| | fever virus |
| 186 | Feline rotavirus |
| 186 | Rotavirus A |
| 186 | Rotavirus C |
| 187 | Equine rotavirus |
| 187 | Rotavirus A |
| 187 | Rotavirus C |
| 188 | New York virus |
| 188 | Sin Nombre virus |
| 189 | Crimean-Congo hemorrhagic |
| | fever virus |
| 190 | Rotavirus A |
| 190 | Rotavirus C |
| 192 | Chimpanzee enterovirus CPS- |
| | 2011 |
| 192 | EIAV-based lentiviral vector |
| 192 | Enterovirus sp. |
| 192 | Human echovirus AMS573 |
| 192 | Human enterovirus A |
| 192 | Human rhinovirus C |
| 192 | Porcine enterovirus B |
| 192 | synthetic construct |
| 192 | uncultured enterovirus |
| 193 | Human immunodeficiency |
| | virus 2 |
| 193 | SIV vector pCLN8 |
| 193 | Simian immunodeficiency |
| | virus |
| 193 | Simian-Human |
| | immunodeficiency virus |
| 193 | synthetic construct |
| 194 | Bundibugyo ebolavirus |
| 195 | Human papillomavirus 121 |
| 196 | Rabbit vesivirus |
| 196 | Steller sea lion vesivirus |
| 196 | Vesicular exanthema of swine |
| | virus |
| 196 | Walrus calicivirus |
| 197 | Alto Paraguay hantavirus |
| 197 | Andes virus |
| 197 | Araucaria virus |
| 197 | Black Creek Canal virus |
| 197 | Catacamas virus |
| 197 | Hantavirus Akomo/RPR/07- |
| | 10028/BRA/2006 |
| 197 | Hantavirus Case Itapua |
| 197 | Hantavirus HMT 08-02 |
| 197 | Hantavirus Monongahela-1 |
| 197 | Hantavirus Olini/RPR/07- |
| | 10091/BRA/2007 |
| 197 | Hantavirus Oln6469 |
| 197 | Hantavirus Oln6470 |
| 197 | Hantavirus Oxyju/RPR/07- |
| | 10056/BRA/2006 |
| 197 | Hantavirus sp. |
| 197 | Hantavirus strain Oln8057 |
| 197 | Huitzilac virus |
| 197 | Itapua hantavirus |
| 197 | Juquitiba virus |
| 197 | Laguna Negra virus |
| 197 | Limestone Canyon virus |
| 197 | Montano virus |
| 197 | Newfound Gap hantavirus |
| 197 | Rio Mamore virus |
| 197 | Sin Nombre virus |
| 198 | Rotavirus A |
| 199 | Human papillomavirus 5 |
| 200 | GB virus A |
| 201 | Equine rotavirus |
| 201 | Feline rotavirus |
| 201 | Rotavirus A |
| 201 | Rotavirus C |
| 201 | Rotavirus sp. |
| 202 | Lymphocytic choriomeningitis |
| | virus |
| 203 | Human papillomavirus 16 |
| 204 | Human papillomavirus 4 |
| 205 | Rotavirus A |
| 206 | Lassa virus |
| 207 | Feline calicivirus |
| 208 | Human papillomavirus 16 |
| 209 | Junin virus |
| 210 | Crimean-Congo hemorrhagic |
| | fever virus |
| 211 | Human norovirus Saitama |
| 211 | Minireovirus |
| 211 | Norwalk virus |
| 211 | Swine norovirus |
| 212 | Equine rotavirus |
| 212 | Rotavirus A |
| 212 | Rotavirus C |
| 213 | Andes virus |
| 213 | Araucaria virus |
| 213 | Cano Delgadito virus |
| 213 | Hantavirus 2036 Biritiba |
| | Mirim |
| 213 | Hantavirus 2062 Biritiba |
| | Mirim |
| 213 | Hantavirus 2063 Biritiba |
| | Mirim |
| 213 | Hantavirus 2066 Biritiba |
| | Mirim |
| 213 | Hantavirus 2070 Biritiba |
| | Mirim |
| 213 | Hantavirus 2071 Biritiba |
| | Mirim |
| 213 | Hantavirus 2072 Biritiba |
| | Mirim |
| 213 | Hantavirus 2306 Biritiba |
| | Mirim |
| 213 | Hantavirus 2336 Biritiba |
| | Mirim |
| 213 | Hantavirus Monongahela-1 |
| 213 | Hantavirus R11 |
| 213 | Hantavirus R34 |
| 213 | Hantavirus sp. Paranoa |
| 213 | Juquitiba virus |
| 213 | Muleshoe virus |
| 213 | New York virus |
| 213 | Newfound Gap hantavirus |
| 213 | Playa de Oro hantavirus |
| 213 | Rio Mamore virus |
| 213 | Sin Nombre virus |
| 214 | Rotavirus A |
| 214 | Rotavirus B |
| 214 | Rotavirus C |
| 214 | Rotavirus sp. |
| 215 | Sapporo virus |
| 216 | Amur virus |
| 216 | Hantaan virus |
| 216 | Hantavirus A9 |
| 216 | Hantavirus CGRn8316 |
| 216 | Hantavirus CGRn9415 |
| 216 | Hantavirus HTN |
| 216 | Hantavirus KY |
| 216 | Hantavirus Liu |
| 216 | Hantavirus XAHu09011 |
| 216 | Hantavirus XAHu09027 |
| 216 | Hantavirus XAHu09041 |
| 216 | Hantavirus XAHu09047 |
| 216 | Hantavirus XAHu09066 |
| 216 | Hantavirus Z10 |
| 216 | Hantavirus Z5 |
| 216 | Soochong virus |
| 217 | Lake Victoria marburgvirus |
| 218 | Dandenong virus |
| 218 | Lymphocytic choriomeningitis |
| | virus |
| 218 | synthetic construct |
| 219 | Bovine respiratory syncytial |
| | virus |
| 219 | Human respiratory syncytial |
| | virus |
| 219 | Respiratory syncytial virus |
| 220 | Japanese encephalitis virus |
| 220 | Koutango virus |
| 220 | Usutu virus |
| 220 | West Nile virus |
| 220 | synthetic construct |
| 221 | Eastern equine encephalitis |
| | virus |
| 221 | Western equine |
| | encephalomyelitis virus |
| 222 | Rotavirus A |
| 224 | Human papillomavirus 18 |
| 225 | Human papillomavirus type |
| | 131 |
| 226 | Human papillomavirus 49 |
| 227 | Murine rotavirus |
| 227 | Rotavirus A |
| 227 | Rotavirus sp. |
| 228 | Rotavirus A |
| 229 | Human papillomavirus 101 |
| 230 | Rotavirus A |
| 231 | Lymphocytic choriomeningitis |
| | virus |
| 232 | Duck hepatitis B virus |
| 232 | Ground squirrel hepatitis virus |
| 232 | Hepatitis B virus |
| 232 | Homo sapiens |
| 232 | Woodchuck hepatitis virus |
| 232 | synthetic construct |
| 232 | uncultured organism |
| 233 | Hepatitis C virus |
| 233 | synthetic construct |
| 234 | Rotavirus A |
| 235 | Rabbit calicivirus Australia 1 |
| | MIC-07 |
| 235 | Rabbit hemorrhagic disease |
| | virus |
| 236 | Human norovirus Saitama |
| 236 | Norwalk virus |
| 237 | Feline rotavirus |
| 237 | Rotavirus A |
| 237 | Rotavirus C |
| 238 | Rotavirus A |
| 239 | Equine rotavirus |
| 239 | Feline rotavirus |
| 239 | Rotavirus A |
| 239 | Rotavirus C |
| 239 | Rotavirus sp. |
| 240 | Rotavirus A |
| 241 | Rotavirus A |
| 242 | Rotavirus A |
| 243 | Rotavirus A |
| 244 | Feline rotavirus |
| 244 | Rotavirus A |
| 244 | Rotavirus sp. |
| 245 | Duck hepatitis B virus |
| 245 | Expression vector pMCG50-S |
| 245 | Ground squirrel hepatitis virus |
| 245 | Hepatitis B virus |
| 245 | Homo sapiens |
| 245 | synthetic construct |
| 246 | El Moro Canyon virus |
| 247 | Murine rotavirus |
| 247 | Rotavirus A |
| 247 | Rotavirus C |
| 247 | Rotavirus sp. |
| 248 | Equine rotavirus |
| 248 | Feline rotavirus |
| 248 | Proteus vulgaris |
| 248 | Rotavirus A |
| 248 | Rotavirus C |
| 248 | Rotavirus sp. |
| 249 | VEEV replicon vector YFV- |
| | C3opt |
| 249 | Venezuelan equine |
| | encephalitis virus |
| 250 | Crimean-Congo hemorrhagic |
| | fever virus |
| 251 | Equine rotavirus |
| 251 | Feline rotavirus |
| 251 | Rotavirus A |
| 251 | Rotavirus B |
| 251 | Rotavirus C |
| 251 | Rotavirus sp. |
| 252 | Rotavirus A |
| 252 | Rotavirus sp. |
| 253 | Vesicular exanthema of swine |
| | virus |
| 254 | Liao ning virus |
| 255 | Amur virus |
| 255 | Hantaan virus |
| 255 | Hantavirus A9 |
| 255 | Hantavirus AH09 |
| 255 | Hantavirus AH211 |
| 255 | Hantavirus CGRn8316 |
| 255 | Hantavirus CGRn9415 |
| 255 | Hantavirus HTN |
| 255 | Hantavirus KY |
| 255 | Hantavirus Liu |
| 255 | Hantavirus XAHu09011 |
| 255 | Hantavirus XAHu09027 |
| 255 | Hantavirus XAHu09041 |
| 255 | Hantavirus XAHu09047 |
| 255 | Hantavirus XAHu09066 |
| 255 | Hantavirus Z10 |
| 255 | Hantavirus Z5 |
| 255 | Soochong virus |
| 256 | Norwalk virus |
| 257 | BK polyomavirus |
| 257 | JC polyomavirus |
| 257 | Simian agent 12 |
| 257 | Simian virus 12 |
| 258 | Feline rotavirus |
| 258 | Rotavirus A |
| 259 | Dengue virus |
| 260 | Rotavirus A |
| 260 | Rotavirus sp. |
| 261 | Lassa virus |
| 262 | Feline rotavirus |
| 262 | Murine rotavirus |
| 262 | Rotavirus A |
| 263 | Human papillomavirus 9 |
| 264 | Cloning vector p119L1e |
| 264 | Homo sapiens |
| 264 | Human papillomavirus 16 |
| 264 | synthetic construct |
| 265 | Crimean-Congo hemorrhagic |
| | fever virus |
| 266 | Lassa virus |
| 266 | Mopeia Lassa reassortant 29 |
| 267 | Crimean-Congo hemorrhagic |
| | fever virus |
| 269 | Chimpanzee enterovirus CPS- |
| | 2011 |
| 269 | EIAV-based lentiviral vector |
| 269 | Enterovirus sp. |
| 269 | Human echovirus AMS573 |
| 269 | Human enterovirus C |
| 269 | Human rhinovirus sp. |
| 269 | Porcine enterovirus B |
| 269 | Simian enterovirus SV6 |
| 269 | Simian picornavirus strain |
| | N125 |
| 269 | synthetic construct |
| 269 | uncultured enterovirus |
| 270 | Feline rotavirus |
| 270 | Rotavirus A |
| 271 | Aids-associated retrovirus |
| 271 | HIV whole-genome vector |
| | AA1305#18 |
| 271 | HIV-1 vector pNL4-3 |
| 271 | Human immunodeficiency |
| | virus 1 |
| 271 | Simian immunodeficiency |
| | virus |
| 271 | synthetic construct |
| 272 | Lassa virus |
| 272 | Mopeia Lassa reassortant 29 |
| 273 | Rotavirus A |
| 274 | Human papillomavirus 61 |
| 275 | Human papillomavirus 61 |
| 276 | Rotavirus A |
| 277 | Equine rotavirus |
| 277 | Rotavirus A |
| 277 | Rotavirus C |
| 277 | Rotavirus sp. |
| 278 | Human norovirus Saitama |
| 278 | Norwalk virus |
| 279 | Human papillomavirus 9 |
| 280 | Feline rotavirus |
| 280 | Murine rotavirus |
| 280 | Rotavirus A |
| 280 | Rotavirus B |
| 280 | Rotavirus C |
| 280 | Rotavirus sp. |
| 281 | Rotavirus A |
| 281 | Rotavirus sp. |
| 282 | Equine rotavirus |
| 282 | Rotavirus A |
| 282 | Rotavirus C |
| 282 | Rotavirus sp. |
| 283 | Rabies virus |
| 283 | Rabies virus-derived |
| | expression vector cSPBN- |
| | 4GFP |
| 284 | Human papillomavirus 5 |
| 285 | Hantaan virus |
| 285 | Hantavirus A9 |
| 285 | Hantavirus KY |
| 285 | Hantavirus Z10 |
| 286 | Human papillomavirus 9 |
| 286 | Macaca fascicularis |
| | papillomavirus |
| 287 | Homo sapiens |
| 287 | Human papillomavirus 18 |
| 288 | Rotavirus A |
| 288 | Rotavirus sp. |
| 289 | Human papillomavirus 90 |
| 290 | Hepatitis C virus |
| 290 | synthetic construct |
| 291 | Japanese encephalitis virus |
| 291 | Koutango virus |
| 291 | West Nile virus |
| 291 | synthetic construct |
| 292 | Equine rotavirus |
| 292 | Feline rotavirus |
| 292 | Rotavirus A |
| 292 | Rotavirus B |
| 292 | Rotavirus C |
| 292 | Rotavirus sp. |
| 293 | Calicivirus isolate 2117 |
| 293 | Canine calicivirus |
| 295 | Human papillomavirus 61 |
| 296 | Russian Spring-Summer |
| | encephalitis virus |
| 296 | Tick-borne encephalitis virus |
| 297 | Hepatitis C virus |
| 297 | synthetic construct |
| 298 | Andes virus |
| 298 | Araucaria virus |
| 298 | Bayou virus |
| 298 | Black Creek Canal virus |
| 298 | Carrizal virus |
| 298 | Catacamas virus |
| 298 | El Moro Canyon virus |
| 298 | Hantavirus Akomo/RPR/07- |
| | 10028/BRA/2006 |
| 298 | Hantavirus Case Itapua |
| 298 | Hantavirus HMT 08-02 |
| 298 | Hantavirus Monongahela-1 |
| 298 | Hantavirus Olini/RPR/07- |
| | 10091/BRA/2007 |
| 298 | Hantavirus Oln6469 |
| 298 | Hantavirus Oln6470 |
| 298 | Hantavirus Oxyju/RPR/07- |
| | 10056/BRA/2006 |
| 298 | Hantavirus YN06-862 |
| 298 | Hantavirus sp. |
| 298 | Hantavirus strain Oln8057 |
| 298 | Huitzilac virus |
| 298 | Itapua hantavirus |
| 298 | Juquitiba virus |
| 298 | Laguna Negra virus |
| 298 | Limestone Canyon virus |
| 298 | Montano virus |
| 298 | Muleshoe virus |
| 298 | New York virus |
| 298 | Newfound Gap hantavirus |
| 298 | Playa de Oro hantavirus |
| 298 | Rio Mamore virus |
| 298 | Rio Segundo virus |
| 298 | Sin Nombre virus |
| 298 | Tula virus |
| 299 | Rotavirus A |
| 299 | Rotavirus C |
| 300 | Lassa virus |
| 300 | Mopeia Lassa reassortant 29 |
| 301 | Hepatitis C virus |
| 301 | synthetic construct |
| 302 | Norwalk virus |
| 302 | Sapporo virus |
| 303 | Human papillomavirus 101 |
| 304 | Eastern equine encephalitis |
| | virus |
| 304 | Fort Morgan virus |
| 304 | Highlands J virus |
| 304 | VEEV replicon vector YFV- |
| | C3opt |
| 304 | Venezuelan equine |
| | encephalitis virus |
| 304 | Western equine |
| | encephalomyelitis virus |
| 305 | YFV replicon vector prME- |
| | def |
| 305 | Yellow fever virus |
| 306 | Equine rotavirus |
| 306 | Feline rotavirus |
| 306 | Rotavirus A |
| 306 | Rotavirus B |
| 306 | Rotavirus C |
| 306 | Rotavirus sp. |
| 307 | Homo sapiens |
| 307 | Human papillomavirus 53 |
| 308 | Hantaan virus |
| 308 | Hantavirus AH09 |
| 308 | Hantavirus KY |
| 309 | Human papillomavirus type |
| | 129 |
| 310 | Sapporo virus |
| 311 | Hantavirus Fusong-Mf-682 |
| 311 | Hantavirus Fusong-Mf-731 |
| 311 | Hantavirus Shenyang-Mf-136 |
| 311 | Hantavirus Yakeshi-Mm-182 |
| 311 | Hantavirus Yakeshi-Mm-31 |
| 311 | Hantavirus Yakeshi-Mm-59 |
| 311 | Hantavirus Yuanjiang-Mf-13 |
| 311 | Hantavirus Yuanjiang-Mf-15 |
| 311 | Hantavirus Yuanjiang-Mf-21 |
| 311 | Hantavirus Yuanjiang-Mf-78 |
| 311 | Hantavirus sp. |
| 311 | Isla Vista virus |
| 311 | Khabarovsk virus |
| 311 | Malacky virus |
| 311 | Prospect Hill virus |
| 311 | Puumala virus |
| 311 | Topografov virus |
| 311 | Tula virus |
| 312 | Feline rotavirus |
| 312 | Rotavirus A |
| 312 | Rotavirus sp. |
| 313 | Equine rotavirus |
| 313 | Feline rotavirus |
| 313 | Rotavirus A |
| 313 | Rotavirus sp. |
| 314 | Rotavirus A |
| 314 | Rotavirus sp. |
| 315 | Feline rotavirus |
| 315 | Rotavirus A |
| 315 | Rotavirus sp. |
| 316 | Human papillomavirus 5 |
| 317 | Feline rotavirus |
| 317 | Rotavirus A |
| 317 | Rotavirus C |
| 317 | Rotavirus sp. |
| 317 | synthetic construct |
| 318 | Feline rotavirus |
| 318 | Human rotavirus HRUKM I |
| 318 | Rotavirus A |
| 318 | Rotavirus C |
| 318 | Rotavirus sp. |
| 318 | synthetic construct |
| 319 | Rotavirus A |
| 320 | Rotavirus A |
| 320 | Rotavirus sp. |
| 321 | Rotavirus A |
| 322 | Human papillomavirus 96 |
| 323 | Rotavirus A |
| 324 | Rotavirus A |
| 324 | Rotavirus C |
| 325 | Rotavirus A |
| 325 | Rotavirus sp. |
| 326 | Human immunodeficiency |
| | virus 1 |
| 326 | Simian immunodeficiency |
| | virus |
| 327 | Rotavirus A |
| 328 | Duck hepatitis A virus |
| 329 | Hantaan virus |
| 329 | Hantavirus KY |
| 329 | Hantavirus Thailand 741 |
| 329 | Seoul virus |
| 329 | Thailand virus |
| 330 | Lymphocytic choriomeningitis |
| | virus |
| 331 | Equine rotavirus |
| 331 | Murine rotavirus |
| 331 | Proteus vulgaris |
| 331 | Rotavirus A |
| 331 | Rotavirus C |
| 331 | Rotavirus sp. |
| 332 | Eyach virus |
| 333 | Lymphocytic choriomeningitis |
| | virus |
| 334 | Rotavirus A |
| 335 | Crimean-Congo hemorrhagic |
| | fever virus |
| 336 | Equine rotavirus |
| 336 | Rotavirus A |
| 337 | Hantavirus Yakeshi-Mm-182 |
| 337 | Hantavirus Yakeshi-Mm-31 |
| 337 | Hantavirus Yakeshi-Mm-59 |
| 337 | Hantavirus sp. |
| 337 | Isla Vista virus |
| 337 | Khabarovsk virus |
| 337 | Malacky virus |
| 337 | Prairie vole hantavirus |
| 337 | Prospect Hill virus |
| 337 | Puumala virus |
| 337 | Topografov virus |
| 337 | Tula virus |
| 338 | Omsk hemorrhagic fever virus |
| 338 | Tick-borne encephalitis virus |
| 339 | Lymphocytic choriomeningitis |
| | virus |
| 339 | synthetic construct |
| 340 | Feline rotavirus |
| 340 | Rotavirus A |
| 340 | Rotavirus C |
| 340 | Rotavirus sp. |
| 341 | Human papillomavirus 90 |
| 342 | Amur virus |
| 342 | Hantaan virus |
| 342 | Hantavirus KY |
| 342 | Hantavirus XAHu09011 |
| 342 | Hantavirus XAHu09027 |
| 342 | Hantavirus XAHu09066 |
| 342 | Hantavirus Z10 |
| 342 | Puumala virus |
| 342 | Seoul virus |
| 342 | Tula virus |
| 343 | Equine rotavirus |
| 343 | Feline rotavirus |
| 343 | Murine rotavirus |
| 343 | Rotavirus A |
| 343 | Rotavirus C |
| 343 | Rotavirus sp. |
| 343 | Shuttle vector pMV361- |
| | Edim6 |
| 345 | Rotavirus A |
| 346 | Norwalk virus |
| 347 | Rotavirus A |
| 348 | Human papillomavirus 5 |
| 349 | Langat virus |
| 349 | Louping ill virus |
| 349 | Omsk hemorrhagic fever virus |
| 349 | Royal Farm virus |
| 349 | Tick-borne encephalitis virus |
| 350 | Rotavirus A |
| 351 | Rotavirus A |
| 352 | California encephalitis virus |
| 353 | Sapporo virus |
| 354 | Amur virus |
| 354 | Hantaan virus |
| 354 | Hantavirus KY |
| 354 | Hantavirus Liu |
| 354 | Hantavirus Z10 |
| 354 | Soochong virus |
| 355 | Rotavirus A |
| 356 | Cloning vector pDBR |
| 356 | HIV whole-genome vector |
| | AA1305#18 |
| 356 | HIV-1 vector pNL4-3 |
| 356 | Human immunodeficiency |
| | virus 1 |
| 356 | Lentiviral transfer vector |
| | pFTM3GW |
| 356 | Lentivirus shuttle vector |
| | pLV.FLPe |
| 356 | Self-inactivating lentivirus |
| | vector pLV.C-EF1a.cyt- |
| | bGal.dCpG |
| 356 | Shuttle vector |
| | pLV.hMyoD.eGFP |
| 356 | Simian immunodeficiency |
| | virus |
| 356 | Simian-Human |
| | immunodeficiency virus |
| 356 | synthetic construct |
| 357 | Amur virus |
| 357 | Hantaan virus |
| 357 | Hantavirus A9 |
| 357 | Hantavirus CGRn8316 |
| 357 | Hantavirus CGRn9415 |
| 357 | Hantavirus HTN |
| 357 | Hantavirus KY |
| 357 | Hantavirus Liu |
| 357 | Hantavirus XAHu09011 |
| 357 | Hantavirus XAHu09027 |
| 357 | Hantavirus XAHu09041 |
| 357 | Hantavirus XAHu09047 |
| 357 | Hantavirus XAHu09066 |
| 357 | Hantavirus Z10 |
| 357 | Hantavirus Z5 |
| 357 | Seoul virus |
| 357 | Soochong virus |
| 358 | Rotavirus A |
| 358 | Rotavirus sp. |
| 359 | Rotavirus A |
| 359 | Rotavirus sp. |
| 360 | GB virus A |
| 361 | Rotavirus A |
| 362 | Influenza C virus |
| 363 | Influenza B virus |
| 364 | Influenza A virus |
| 365 | Dhori virus |
| 366 | Influenza C virus |
| 367 | Influenza A virus |
| 368 | Thogoto virus |
| 369 | Dhori virus |
| 370 | Influenza B virus |
| 371 | Influenza C virus |
| 372 | Infectious salmon anemia |
| | virus |
| 373 | Influenza A virus |
| 374 | Influenza C virus |
| 375 | Influenza A virus |
| 376 | Expression vector |
| | pPICK9KH1N1HA |
| 376 | Influenza A virus |
| 376 | unidentified influenza virus |
| 377 | Influenza A virus |
| 378 | Influenza A virus |
| 379 | Infectious salmon anemia |
| | virus |
| 380 | Influenza A virus |
| 380 | unidentified influenza virus |
| 381 | Influenza A virus |
| 382 | Influenza A virus |
| 383 | Influenza A virus |
| 383 | unidentified influenza virus |
| 384 | Influenza A virus |
| 385 | Influenza A virus |
| 386 | Influenza A virus |
| 387 | Influenza A virus |
| 387 | unidentified influenza virus |
| 388 | Influenza A virus |
| 389 | Influenza A virus |
| 390 | Influenza A virus |
| 391 | Influenza C virus |
| 392 | Influenza A virus |
| 393 | Influenza A virus |
| 393 | synthetic construct |
| 394 | Infectious salmon anemia |
| | virus |
| 395 | Infectious salmon anemia |
| | virus |
| 396 | Influenza A virus |
| 397 | Influenza A virus |
| 398 | Influenza A virus |
| 399 | Expression vector |
| | pPICK9KH1N1HA |
| 399 | Influenza A virus |
| 399 | unidentified influenza virus |
| 400 | Dicistronic cloning vector |
| | pXL-Id |
| 400 | Fowl plague virus |
| 400 | Influenza A virus |
| 400 | unidentified influenza virus |
| 401 | Influenza A virus |
| 402 | Influenza A virus |
| 403 | Influenza A virus |
| 404 | Influenza A virus |
| 405 | Influenza A virus |
| 406 | Influenza A virus |
| 406 | unidentified influenza virus |
| 407 | Influenza A virus |
| 407 | Influenza B virus |
| 407 | synthetic construct |
| 407 | unidentified influenza virus |
| 408 | Influenza A virus |
| 409 | Influenza A virus |
| 410 | Influenza A virus |
| 411 | Influenza A virus |
| 411 | unidentified influenza virus |
| 412 | Influenza A virus |
| 413 | Influenza A virus |
| 414 | Influenza A virus |
| 415 | Influenza A virus |
| 416 | Fowl plague virus |
| 416 | Influenza A virus |
| 417 | Influenza A virus |
| 418 | Dicistronic cloning vector |
| | pXL-Id |
| 418 | Fowl plague virus |
| 418 | Influenza A virus |
| 418 | unidentified influenza virus |
| 419 | Influenza A virus |
| 420 | Influenza B virus |
| 421 | Infectious salmon anemia |
| | virus |
| 422 | Infectious salmon anemia |
| | virus |
| 423 | Influenza A virus |
| 423 | unidentified influenza virus |
| 424 | Infectious salmon anemia |
| | virus |
| 425 | Influenza A virus |
| 425 | unidentified influenza virus |
| 426 | Thogoto virus |
| 427 | Influenza A virus |
| 428 | Influenza B virus |
| 429 | Influenza A virus |
| 429 | unidentified influenza virus |
| 430 | Influenza A virus |
| 431 | Influenza C virus |
| 432 | Infectious salmon anemia |
| | virus |
| 433 | Influenza A virus |
| 433 | Influenza B virus |
| 434 | Influenza A virus |
| 435 | Influenza A virus |
| 435 | synthetic construct |
| 436 | Influenza A virus |
| 436 | synthetic construct |
| 437 | Influenza A virus |
| 438 | Influenza A virus |
| 438 | unidentified influenza virus |
| 439 | Influenza A virus |
| 439 | unidentified influenza virus |
| 440 | Influenza A virus |
| 440 | unidentified influenza virus |
| 441 | Influenza A virus |
| 442 | Influenza A virus |
| 443 | Influenza A virus |
| 443 | unidentified influenza virus |
| 444 | Influenza A virus |
| 445 | Influenza A virus |

Over a range of 133,263, table 11 shows a correspondence between probes having SEQ ID NO's 446-133,263 and a family of species that can be detected.

TABLE 11
Families of bacterial, viral, and flu species which can be detected
by probes corresponding to SEQ ID NO's 1-133, 263.

| Family | Start_SEQ_ID_NO | End_SEQ_ID_NO |
|---|---|---|
| Acetobacteraceae | 446 | 522 |
| Acholeplasmataceae | 523 | 550 |
| Aeromonadaceae | 551 | 580 |
| Alcaligenaceae | 581 | 778 |
| Anaplasmataceae | 779 | 816 |
| Bacillaceae | 817 | 1207 |
| Bacteroidaceae | 1208 | 1264 |
| Bartonellaceae | 1265 | 1279 |
| Bdellovibrionaceae | 1280 | 1430 |
| Bifidobacteriaceae | 1431 | 1460 |
| Bradyrhizobiaceae | 1461 | 1725 |
| Brevibacteriaceae | 1726 | 1740 |
| Brucellaceae | 1741 | 1769 |
| Burkholderiaceae | 1770 | 1991 |
| Campylobacteraceae | 1992 | 2031 |
| Cardiobacteriaceae | 2032 | 2046 |
| Caulobacteraceae | 2047 | 2061 |
| Cellulomonadaceae | 2062 | 2086 |
| Chlamydiaceae | 2087 | 2156 |
| Clostridiaceae | 2157 | 2357 |
| Comamonadaceae | 2358 | 2442 |
| Corynebacteriaceae | 2443 | 2612 |
| Coxiellaceae | 2613 | 2657 |
| Enterobacteriaceae | 2658 | 2992 |
| Enterococcaceae | 2993 | 3033 |
| Francisellaceae | 3034 | 3061 |
| Fusobacteriaceae | 3062 | 3076 |
| Gordoniaceae | 3077 | 3091 |
| Halomonadaceae | 3092 | 3106 |
| Helicobacteraceae | 3107 | 3203 |
| Lachnospiraceae | 3204 | 3218 |
| Lactobacillaceae | 3219 | 3434 |
| Legionellaceae | 3435 | 3475 |
| Leptospiraceae | 3476 | 3500 |
| Leuconostocaceae | 3501 | 3541 |
| Listeriaceae | 3542 | 3709 |
| Micrococcaceae | 3710 | 3739 |
| Moraxellaceae | 3740 | 3802 |
| Mycobacteriaceae | 3803 | 4016 |
| Mycoplasmataceae | 4017 | 4175 |
| Neisseriaceae | 4176 | 4200 |
| Nocardiaceae | 4201 | 4250 |
| Oxalobacteraceae | 4251 | 4265 |
| Parachlamydiaceae | 4266 | 4280 |
| Pasteurellaceae | 4281 | 4373 |
| Peptococcaceae | 4374 | 4432 |
| Piscirickettsiaceae | 4433 | 4447 |
| Pseudomonadaceae | 4448 | 4545 |
| Rickettsiaceae | 4546 | 4649 |
| Staphylococcaceae | 4650 | 4823 |
| Streptococcaceae | 4824 | 5053 |
| Vibrionaceae | 5054 | 5183 |
| Spirochaetaceae | 5184 | 5402 |
| Porphyromonadaceae | 5403 | 5431 |
| Prevotellaceae | 5432 | 5446 |
| Propionibacteriaceae | 5447 | 5460 |
| Streptomycetaceae | 5461 | 5722 |
| Adenoviridae | 5723 | 5808 |
| Alloherpesviridae | 5809 | 5823 |
| Anelloviridae | 5824 | 5972 |
| Arenaviridae | 5973 | 6303 |
| Arteriviridae | 6304 | 6353 |
| Asfarviridae | 6354 | 6359 |
| Astroviridae | 6360 | 6447 |
| Birnaviridae | 6448 | 6525 |
| Bornaviridae | 6526 | 6532 |
| Bunyaviridae | 6533 | 7290 |
| Caliciviridae | 7291 | 7553 |
| Circoviridae | 7554 | 7688 |
| Coronaviridae | 7689 | 7797 |
| Filoviridae | 7798 | 7827 |
| Flaviviridae | 7828 | 8476 |
| Hepadnaviridae | 8477 | 8607 |
| Hepeviridae | 8608 | 8770 |
| Herpesviridae | 8771 | 8921 |
| Iridoviridae | 8922 | 8950 |
| Nodaviridae | 8951 | 9020 |
| Orthomyxoviridae | 9021 | 10206 |
| Papillomaviridae | 10207 | 10690 |
| Paramyxoviridae | 10691 | 10980 |
| Parvoviridae | 10981 | 11127 |
| Picobirnaviridae | 11128 | 11134 |
| Picornaviridae | 11135 | 12036 |
| Polyomaviridae | 12037 | 12104 |
| Poxviridae | 12105 | 12153 |
| Reoviridae | 12154 | 14627 |
| Retroviridae | 14628 | 15559 |
| Rhabdoviridae | 15560 | 15759 |
| Roniviridae | 15760 | 15765 |
| Togaviridae | 15766 | 15861 |
| Adenoviridae | 15862 | 15958 |
| Alloherpesviridae | 15959 | 15960 |
| Anelloviridae | 15961 | 16096 |
| Arenaviridae | 16097 | 16175 |
| Arteriviridae | 16176 | 16212 |
| Astroviridae | 16214 | 16247 |
| Birnaviridae | 16248 | 16286 |
| Bornaviridae | 16287 | 16294 |
| Bunyaviridae | 16295 | 16462 |
| Caliciviridae | 16463 | 16637 |
| Circoviridae | 16638 | 16731 |
| Coronaviridae | 16732 | 16794 |
| Filoviridae | 16795 | 16808 |
| Flaviviridae | 16809 | 17224 |
| Hepadnaviridae | 17225 | 17331 |
| Hepeviridae | 17332 | 17436 |
| Herpesviridae | 17437 | 17494 |
| Iridoviridae | 17495 | 17503 |
| Nodaviridae | 17504 | 17544 |
| Orthomyxoviridae | 17545 | 17929 |
| Papillomaviridae | 17930 | 18248 |
| Paramyxoviridae | 18249 | 18376 |
| Parvoviridae | 18377 | 18468 |
| Picobirnaviridae | 18469 | 18471 |
| Picornaviridae | 18472 | 18961 |
| Polyomaviridae | 18962 | 18994 |
| Poxviridae | 18995 | 19022 |
| Reoviridae | 19023 | 19916 |
| Retroviridae | 19917 | 20371 |
| Rhabdoviridae | 20372 | 20513 |
| Roniviridae | 20514 | 20517 |
| Togaviridae | 20518 | 20592 |
| Adenoviridae | 20593 | 21733 |
| Arenaviridae | 21734 | 24355 |
| Arteriviridae | 24356 | 24634 |
| Asfarviridae | 24635 | 24684 |
| Astroviridae | 24685 | 25023 |
| Birnaviridae | 25024 | 25459 |
| Bornaviridae | 25460 | 25512 |
| Bunyaviridae | 25513 | 38302 |
| Caliciviridae | 38303 | 40182 |
| Circoviridae | 40183 | 40876 |
| Coronaviridae | 40877 | 41793 |
| Flaviviridae | 41794 | 44589 |
| Filoviridae | 44590 | 44832 |
| Hepeviridae | 44833 | 45133 |
| Hepadnaviridae | 45134 | 45509 |
| Herpesviridae | 45510 | 47218 |
| Iridoviridae | 47219 | 47568 |
| Nodaviridae | 47569 | 48274 |
| Orthomyxoviridae | 48275 | 91627 |
| Papillomaviridae | 91628 | 95180 |
| Paramyxoviridae | 95181 | 97035 |
| Parvoviridae | 97036 | 98745 |
| Picornaviridae | 98746 | 101837 |
| Polyomaviridae | 101838 | 102612 |
| Poxviridae | 102613 | 103348 |
| Reoviridae | 103349 | 124732 |
| Retroviridae | 124733 | 130081 |
| Rhabdoviridae | 130082 | 131448 |
| Roniviridae | 131449 | 131970 |
| Togaviridae | 131971 | 133263 |

### Example 15

### Detection Probability of a Target Based on Empirical Means

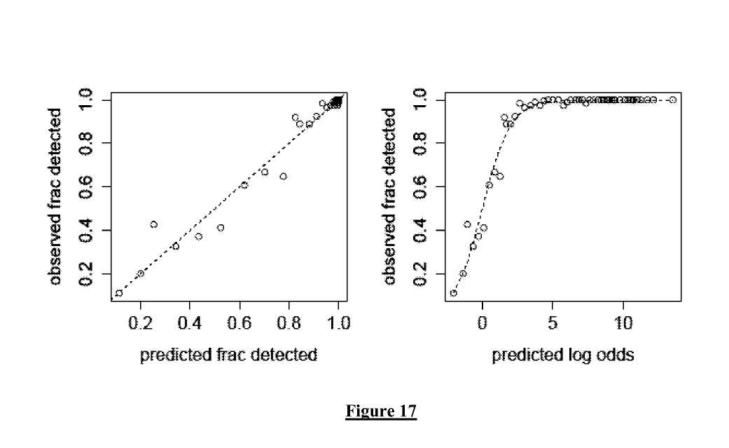

Using the empirical data of previous array versions, predictors can be formulated to determine the detection probability of a target probe (see Example 13). A linear predictor can be derived from parameters with desired predictive values such as an alignment score, a predicted Tₘ of the probe to its matching target sequence, and the start position of the match on the probe also known as a hit start. An exemplary alignment score is a BLAST bit score. For example, FIG. 17 shows plots, for a particular array experiment, in which the left panel of FIG. 17 shows observed vs predicted detected fraction, in 50 bins of approximately 280 probe-target pairs each, and the right panel of FIG. 17 observed fraction vs predicted log-odds from the logistic regression fit, over the same bins. In logistic regression the log-odds is a linear combination of the predictive variables, which in the exemplary case of FIG. 17 were the BLAST bitscore, melting temperature over matching bases, and the start position of the target alignment in the probe sequence.

An exemplary equation of detection probability based on common parameters across all arrays is derived from linear predictors derived from an alignment score, a predicted Tm of the probe to its matching target sequence, and the start position of the match on the probe is:

Detection probability of being present=1−1/(1+exp(−8.684612924+0.163626821×blast bit score+0.001882077×hit start on probe−0.029316625×predicted Tm of matching sequence to probe)),

wherein the predicted Tₘ of matching sequence is calculated as

Tₘ=69.4+(41×number of G and C bases in probe−600.0)/(probe length−number of mismatches between probe and target).

Exemplary equations, such as the one above, can be calculated for different brands or makes of arrays. For example, the equation above was derived from data and further use of Nimblegen arrays. A person of ordinary skill can use the same or similar method to derive an equation of detection probability but the parameters can be different.

### Example 16

### Probes for an Array of a 360K Design

A detection microarray for targeting pathogens in a cost effective format (388K Nimblegen format) according to embodiments of the present disclosure is now described. The following example describes the design of a microarray for detecting viruses, bacteria, fungi, archaea, and protozoa of importance to humans in term of health, agriculture, and economy. The array includes 361,863 probes from all families. Each oligonucleotide probe for detection of at least one target in a target group comprises a sequence selected from a group consisting of SEQ ID NO's 133,264-491,462 and 495,659-534,156, Detection can occur in combination with at least four other oligonucleotide probes selected from the group consisting of SEQ ID NO's 133,264-491,462; and said target is a microorganism, such a bacterium, virus, protozoa, archaeon, or fungus.

Complete viral, bacterial, fungal, archaeal, and protozoan genome/segment/plasmid sequences were gathered from publicly available sites (Genbank, JCVI, IMG, etc.) and from collaborators (CDC, USDA, USAMRIID, NBACC, LANL, etc), and were organized by family. Regions that were specific to a family were identified in which there were no regions longer than 19 bases (or k=19, where k represents the number of bases) or under relaxed conditions where k=20, 21, or 22 that matched viruses, bacteria, fungi, archaea, and protozoa genomes not in the target family, the human genome, the RepBase repeat database, or the SILVA ribosomal RNA database.

From these family-unique regions, candidate probes were identified to meet desired ranges for length (40-60 bases), Tm, entropy, GC %, and other thermodynamic and sequence features to the extent possible given the unique sequence. Detailed thermodynamic parameters are described in reference 28. The desired parameter ranges were relaxed as needed when there were too few probes for a target sequence including raising the length k for calculating family specific regions to 20, 21, or 22 if necessary, as Applicant's aimed at having at least 30 probes per target sequence selected from the conservation favoring probes and at least 5 probes per target sequence selected from the discriminating probes, although there was variation around these numbers due to differences in target length and uniqueness.

Candidate probes were clustered and ranked within each family by the number of targets detected, and a greedy algorithm, as described was used to select a probe set to detect as many of the targets as possible with the fewest probes. Conserved and discriminating probes were chosen as candidate probes.

Uniqueness for bacterial, viral, fungal, and archaeal sequences was calculated relative to all bacterial, viral, fungal, archaeal, and protozoa families, the human genome, repeat sequences in RepBase, and rRNA in the SILVA database. Within the protozoa, uniqueness was calculated relative to bacterial, viral, fungal, and archael sequences, the human genome, repeat sequences in RepBase, and rRNA in the SILVA database.

All 131 viral families and family unclassified groups of sequences were included, as listed in 0085. 338 bacteria families or groups of family unclassified sequences, 37 archaea, 101 fungi. Protozoa were not subgrouped by family. In particular, oligonucleotide probes comprising sequences from a group consisting of SEQ ID NO's 133,264-141,123 and 495,659-496,378 are directed to the detection of archaea, SEQ ID NO's 141, 125-267-772 and 496,379-512,129 are directed to the detection of bacteria, SEQ ID NO's 267,773-286,565 and 512,130-514,809 are directed to the detection of fungi, SEQ ID NO's 286,566-297,255 and 514,810-515,886 are directed to the detection of protozoa, and SEQ ID NO's 297,256-486,081 and 515,887-534,156 are directed to the detection of viruses. The probes described in this exemplary design can be arranged in an array, such as a microarray described in Example 12. Controls can be incorporated into arrays such as random negative controls and/or Thermotoga positive controls.

### Example 17

### Probes for a Clinical Microbial Array from 135K Design

The following example describes a microarray for microbial detection of organisms from families known to infect vertebrates. A detection microarray targeting clinically relevant pathogens in a cost effective format (135K Nimblegen format) was designed. A subset of the families in v5 were downselected for inclusion in a Clinical 135K array, designing probes for clinically relevant viral, bacterial, and fungal families or family unclassified groups with members known to infect vertebrate hosts. For this design, the goal was 15 conserved probes per sequence and 2 discriminating probes per sequence with no Primux-designed probes. Some probes of the 135K design overlap with probes of the 360K design. This smaller design allows testing at lower cost per sample than the larger design. Vertebrate infecting bacterial, viral, and fungal families or groups were selected based on extensive literature (PubMed), web searches, and lists compiled by the International Committee on Taxonomy of Viruses and are available from virology.net/Big_Virology/BVHostList.html#Vertebrates to determine whether any members of a family have been found to infect vertebrates or were involved in clinical infections, and all members of a family were included even if only some of them were vertebrate-infecting. Each oligonucleotide probe for detection of at least one target in a target group comprises a sequence selected from a group consisting of SEQ ID NO's 491,463-495,658 and 534,157-661,081, where said detection occurs in combination with at least four other oligonucleotide probes selected from the group consisting of SEQ ID NO's 491,463-495,658 and 534,157-661,081; and said target is a microorganism. In particular, oligonucleotide probes comprising sequences from a group consisting of SEQ ID NO's 491,463-491,510 and 650,746-653,508 are directed to the detection of archaea, SEQ ID NO's 491,511-492,337 and 615,629-650,745 are directed to the detection of bacteria, SEQ ID NO's 492,338-492,436 and 653,509-657,360 are directed to the detection of fungi, SEQ ID NO's 492,437-492,544 and 657,361-661,081 are directed to the detection of protozoa, and SEQ ID NO's 492,545-495,658 and 534,157-615,628 are directed to the detection of viruses. In particular, oligonucleotide probes comprising sequences from a group consisting of SEQ ID NO's 491,463-495,658 are not present in the 360K set.

A set of 84,586 viral probes were designed for this array including the following 38 viral families or family unclassified groups:

Adenoviridae, Alloherpesviridae, Anelloviridae, Arenaviridae, Arteriviridae, Asfarviridae, Astroviridae, Birnaviridae, Bornaviridae, Bunyaviridae, Caliciviridae, Circoviridae, Coronaviridae, Filoviridae, Flaviviridae, Hepadnaviridae, Hepeviridae, Herpesviridae, Iridoviridae, Nodaviridae, Orthomyxoviridae, Papillomaviridae, Paramyxoviridae, Parvoviridae, Picobirnaviridae, Picornaviridae, Polyomaviridae, Poxyiridae, Reoviridae, Retroviridae, Rhabdoviridae, Togaviridae, Deltavirus, Mononegavirales, Nidovirales, Picornavirales, unclassified_dsDNA_viruses, unclassified_ssDNA_viruses, unclassified_viruses

A set of 35,944 bacterial probes were designed for this array including the following 140 bacterial families or family unclassified groups:

Acetobacteraceae, Acholeplasmataceae, Acidaminococcaceae, Actinomycetaceae, Actinosynnemataceae, Aerococcaceae, Aeromonadaceae, Alcaligenaceae, Anaeroplasmataceae, Anaplasmataceae, Bacillaceae, Bacteroidaceae, Bartonellaceae, Bdellovibrionaceae, Bifidobacteriaceae, Brachyspiraceae, Bradyrhizobiaceae, Brevibacteriaceae, Brucellaceae, Burkholderiaceae, Campylobacteraceae, Cardiobacteriaceae, Carnobacteriaceae, Catabacteriaceae, Caulobacteraceae, Cellulomonadaceae, Chlamydiaceae, Clostridiaceae, Clostridiales_Family_XI, Clostridiales_Family_XII, Clostridiales_Family_XIII, Clostridiales_Family_XIV, Clostridiales_Family_XV, Clostridiales_Family_XVI, Clostridiales_Family_XVII, Clostridiales_Family_XVIII, Comamonadaceae, Coriobacteriaceae, Corynebacteriaceae, Coxiellaceae, Criblamydiaceae, Cyclobacteriaceae, Deferribacteraceae, Dermabacteraceae, Dermacoccaceae, Dermatophilaceae, Desulfohalobiaceae, Desulfomicrobiaceae, Desulfovibrionaceae, Dietziaceae, Enterobacteriaceae, Enterococcaceae, Entomoplasmataceae, Erysipelotrichaceae, Erythrobacteraceae, Eubacteriaceae, Family_X, Family_XVII, Fibrobacteraceae, Flavobacteriaceae, Francisellaceae, Fusobacteriaceae, Gordoniaceae, Halomonadaceae, Helicobacteraceae, Herpetosiphonaceae, Intrasporangiaceae, Jonesiaceae, Lachnospiraceae, Lactobacillaceae, Legionellaceae, Leptospiraceae, Leuconostocaceae, Listeriaceae, Methylobacteriaceae, Micrococcaceae, Moraxellaceae, Mycobacteriaceae, Mycoplasmataceae, Neisseriaceae, Nocardiaceae, Oxalobacteraceae, Parachlamydiaceae, Pasteurellaceae, Peptococcaceae, Peptostreptococcaceae, Piscirickettsiaceae, Porphyromonadaceae, Prevotellaceae, Propionibacteriaceae, Pseudomonadaceae, Pseudonocardiaceae, Rickettsiaceae, Rikenellaceae, Ruminococcaceae, Segniliparaceae, Simkaniaceae, Sphingomonadaceae, Spirillaceae, Spirochaetaceae, Spiroplasmataceae, Sporolactobacillaceae, Staphylococcaceae, Streptococcaceae, Streptomycetaceae, Succinivibrionaceae, Sutterellaceae, Synergistaceae, Tsukamurellaceae, Veillonellaceae, Verrucomicrobia_subdivision_{—}3, Verrucomicrobiaceae, Vibrionaceae, Victivallaceae, Waddliaceae, Xanthomonadaceae, Bhargavaea, Blautia, Burkholderiales, Campylobacterales, Candidatus_Midichloria, Chroococcales, Clostridiales, Epulopiscium, Fangia, Flavobacteriales, Gemella, Microcystis, Oscillatoria, Pseudoflavonifractor, Rickettsiales, Thiotrichales, Tropheryma, Verrucomicrobiales, Vibrionales, candidate_division_TM7, environmental_samples, unclassified_Bacteria, unclassified_Bacteroidetes, unclassified_pseudomonads

A set of 3,951 fungal probes were designed for this array including the following 16 fungi families:

Ajellomycetaceae, Arthrodermataceae, Chaetomiaceae, Debaryomycetaceae, Enterocytozoonidae, Malasseziaceae, Metschnikowiaceae, Mortierellaceae, Mucoraceae, Onygenaceae, Pleosporaceae, Pneumocystidaceae, Schizophyllaceae, Tremellaceae, Trichocomaceae, Unikaryonidae

A set of 2,811 archaeal probes were designed for this array to include all archael families (37 families). A set of 3,829 protozoan probes were designed for this array to include all protozoan families (36 families). The probes described in this exemplary design can be arranged in an array, such as a microarray described in Example 12. Controls can be incorporated into arrays such as random negative controls and/or Thermotoga positive controls.

### Example 18

### A Set of Well-Performing Probes

Of the 135K viral and bacterial probes identified in Example 12, a set of 10 well-performing probes with respect to a target genome sequence was selected shown below in Table 12. In this exemplary embodiment, probes were selected by looking at experimental results from hybridizing the 135 array with samples containing the indicated diseases/infections, such as cholera, or pathogens, such as acinetobacter. Probes selected were perfect matches to the target genome and had a high signal on the array (such as log 2 intensity >15).

TABLE 12
Set of well-performing probes with respect to a target genome sequence.

| | | Location in |
|---|---|---|
| | | target |
| | | genome |
| Probe sequence | Target genome sequence | sequence |
| SEQ ID 5071: | Vibrio cholerae M66-2 | 1898262 |
| GCGGCGGTTTCCTTGGTTGTATCGTAG | chromosome I, complete | |
| CGGGCTTCATCGCCGGTGGTGTGGTAT | genome | |
| TCCAAC | | |
| SEQ ID 5076: | Vibrio cholerae M66-2 | 1518725 |
| GGGCGAAGGGGAGTTTACGGCGGTGA | chromosome I, complete | |
| ACTGGGGCACATCGAATGTGGGCATTA | genome | |
| AAGTCGG | | |
| SEQ ID 5075: | Vibrio cholerae M66-2 | 1520278 |
| CCCGTGAAGATGTTTGACGTGCCTGTT | chromosome I, complete | |
| GCGTAGAACACATCATCGCCTCGTCCG | genome | |
| CCCCAG | | |
| SEQ ID 5072: | Vibrio cholerae M66-2 | 1575043 |
| GGTGGAGTGGCAAATACGCGCTTGGT | chromosome I, complete | |
| GGTCAACGTTGTTGGTGCCCCACAGGG | genome | |
| AAGCCAT | | |
| SEQ ID 5059: | Vibrio cholerae M66-2 | 97708 |
| CCAAGTGGGTCTGCCACTGGAAGGGA | chromosome II, complete | |
| TTGCGCTGATCATGGGTGTCGACCGTC | genome | |
| TACTGGA | | |
| SEQ ID 3789: | Acinetobacter baumannii, | 2840756 |
| GAACCGACCATCCCGCGCCAACCGAC | complete genome | |
| CAGACCTACTTTCATGTCATTTTGCCTC | | |
| GGTGCG | | |
| SEQ ID 35068: | Rift Valley fever virus strain | 2645 |
| GGGAGCATCATCTAGCCGTTTCACAAA | OS-1 segment M, complete | |
| CTGGGGCTCAGTTAGCCTCTCACTGGA | sequence | |
| TGCAGA | | |
| SEQ ID 43291: | Dengue virus type 4 strain | 7948 |
| GGGTTGACGTGTTCTACAAACCCACTG | ThD4_0087_77, complete | |
| AGCAAGTGGACACCCTGCTCTGTGATA | genome | |
| TCGGGG | | |
| SEQ ID 100138: | Foot-and-mouth disease virus - | 8109 |
| GAGATACCAAGCTACAGATCACTTTAC | type Asia 1 isolate IND 182- | |
| CTGCGTTGGGTGAACGCCGTGTGCGGT | 02, complete genome | |
| GACGCA | | |
| SEQ ID 2809: | Yersinia pestis biovar | 362737 |
| CGGGAGCGTTTTAAGCAGGTTTCCGGA | Orientalis str. MG05-1020, | |
| CAGGCGAAAGCTGCCAACAGACAGAG | whole genome | |
| CTGTGGC | | |

The examples set forth above are provided to give those of ordinary skill in the art a complete disclosure and description of how to make and use the embodiments of the pan microbial detection arrays, methods and systems of the disclosure, and are not intended to limit the scope of what the inventors regard as their disclosure. Modifications of the above-described modes for carrying out the disclosure that are obvious to persons of skill in the art are intended to be within the scope of the following claims.

It is to be understood that the disclosures are not limited to particular technical applications or fields of study, which can, of course, vary. It is also to be understood that the terminology used herein is for the purpose of describing particular embodiments only, and is not intended to be limiting. As used in this specification and the appended claims, the singular forms “a,” “an,” and “the” include plural referents unless the content clearly dictates otherwise. The term “plurality” includes two or more referents unless the content clearly dictates otherwise. Unless defined otherwise, all technical and scientific terms used herein have the same meaning as commonly understood by one of ordinary skill in the art to which the disclosure pertains. All references (including, but not limited to, articles, publications, patent applications and patents), mentioned in the present application are incorporated herein by reference in their entirety.

Further, the sequence listing submitted on compact disc concurrently with the present application in the txt file “IL-12080-P425-USCIP2-Sequence-List-text” (created on May 2, 2013) forms an integral part of the present application and is incorporated herein by reference in its entirety.

Although any methods and materials similar or equivalent to those described herein can be used in the practice for testing of the specific examples of appropriate materials and methods are described herein.

A number of embodiments of the disclosure have been described. Nevertheless, it will be understood that various modifications may be made without departing from the spirit and scope of the present disclosure. Accordingly, other embodiments are within the scope of the following claims.

### LIST OF REFERENCES

[1] Anthony, R. M., Brown, T. J. and French, G. L. (2000) Rapid Diagnosis of Bacteremia by Universal Amplification of 23S Ribosomal DNA Followed by Hybridization to an Oligonucleotide Array, J. Clin. Microbiol., 38, 781-788.
[2] Bollet, C., Grimont, P., Gainnier, M., Geissler, A., Sainty, J. M. and De Micco, P. (1993) Fatal pneumonia due to Serratia proteamaculans subsp. quinovora, J. Clin. Microbiol., 31, 444-445.
[3] Chiu, Charles Y., Rouskin, S., Koshy, A., Urisman, A., Fischer, K., Yagi, S., Schnurr, D., Eckburg, Paul B., Tompkins, Lucy S., Blackburn, Brian G., Merker, Jason D., Patterson, Bruce K., Ganem, D. and DeRisi, Joseph L. (2006) Microarray Detection of Human Parainfluenzavirus 4 Infection Associated with Respiratory Failure in an Immunocompetent Adult, Clinical Infectious Diseases, 43, e71-e76.
[4] Chou, C.-C., Lee, T.-T., Chen, C.-H., Hsiao, H.-Y., Lin, Y.-L., Ho, M.-S., Yang, P.-C. and Peck, K. (2006) Design of microarray probes for virus identification and detection of emerging viruses at the genus level, BMC Bioinformatics, 7, 232.
[5] DeSantis, T., Brodie, E., Moberg, J., Zubieta, I., Piceno, Y. and Andersen, G. (2007) High-Density Universal 16S rRNA Microarray Analysis Reveals Broader Diversity than Typical Clone Library When Sampling the Environment, Microbial Ecology, 53, 371-383.
[6] Giegerich, R., Kurtz, S, and Stoye, J. (2003) Efficient implementation of lazy suffix trees, Software-Practice and Experience, 33, 1035-1049.
[7] Jabado, O. J., Liu, Y., Conlan, S., Quan, P. L., Hegyi, H., Lussier, Y., Briese, T., Palacios, G. and Lipkin, W. I. (2008) Comprehensive viral oligonucleotide probe design using conserved protein regions, Nucl. Acids Res., 36, e3.
[8] Jaing, C., Gardner, S., McLoughlin, K., Mulakken, N., Alegria-Hartman, M., Banda, P., Williams, P., Gu, P., Wagner, M., Manohar, C. and Slezak, T. (2008) A Functional Gene Array for Detection of Bacterial Virulence Elements, PLoS ONE, 3, e2163.
[9] Jin, L.-Q., Li, J.-W., Wang, S.-Q., Chao, F.-H., Wang, X.-W. and Yuan, Z.-Q. (2005) Detection and identificatio of intestinal pathogenic bacteria by hybridization to oligonucleotide microarrays, World J Gastroenterol, 11, 7615-7619.
[10] Kessler, N., Ferraris, 0., Palmer, K., Marsh, W. and Steel, A. (2004) Use of the DNA Flow-Thru Chip, a Three-Dimensional Biochip, for Typing and Subtyping of Influenza Viruses, J. Clin. Microbiol, 42, 2173-2185.
[11] Lin, B., Blaney, K. M., Malanoski, A. P., Ligler, A. G., Schnur, J. M., Metzgar, D., Russell, K. L. and Stenger, D. A. (2007) Using a Resequencing Microarray as a Multiple Respiratory Pathogen Detection Assay, J. Clin. Microbiol., 45, 443-452.
[12] Makarova, K., Slesarev, A., Wolf, Y., Sorokin, A., Mirkin, B., Koonin, E., Pavlov, A., Pavlova, N., Karamychev, V., Polouchine, N., Shakhova, V., Grigoriev, I., Lou, Y., Rohksar, D., Lucas, S., Huang, K., Goodstein, D. M., Hawkins, T., Plengvidhya, V., Welker, D., Hughes, J., Goh, Y., Benson, A., Baldwin, K., Lee, J. H., Dosti, B., Smeianov, V., Wechter, W., Barabote, R., Lorca, G., Alternann, E., Barrangou, R., Ganesan, B., Xie, Y., Rawsthorne, H., Tamir, D., Parker, C., Breidt, F., Broadbent, J., Hutkins, R., O'Sullivan, D., Steele, J., Unlu, G., Saier, M., Klaenhammer, T., Richardson, P., Kozyavkin, S., Weimer, B. and Mills, D. (2006) Comparative genomics of the lactic acid bacteria, Proceedings of the National Academy of Sciences, 103, 15611-15616.
[13] Nakamura, S., Yang, C.-S., Sakon, N., Ueda, M., Tougan, T., Yamashita, A., Goto, N., Takahashi, K., Yasunaga, T., Ikuta, K., Mizutani, T., Okamoto, Y., Tagami, M., Morita, R., Maeda, N., Kawai, J., Hayashizaki, Y., Nagai, Y., Horii, T., Lida, T. and Nakaya, T. (2009) Direct Metagenomic Detection of Viral Pathogens in Nasal and Fecal Specimens Using an Unbiased High-Throughput Sequencing Approach, PLoS ONE, 4, e4219.
[14] Palacios, G., Quan, P.-L., Jabado, O., Conlan, S., Hirschberg, D. and Liu Y, e.a. (2007) Panmicrobial oligonucleotide array for diagnosis of infectious diseases, Emerg Infect Dis 13, http://www.cdc.govincidod/EID/13/11/73.htm.
[15] Quan, P.-L., Palacios, G., Jabado, O. J., Conlan, S., Hirschberg, D. L., Pozo, F., Jack, P. J. M., Cisterna, D., Renwick, N., Hui, J., Drysdale, A., Amos-Ritchie, R., Baumeister, E., Savy, V., Lager, K. M., Richt, J. A., Boyle, D. B., Garcia-Sastre, A., Casas, I., Perez-Brena, P., Briese, T. and Lipkin, W. I. (2007) Detection of Respiratory Viruses and Subtype Identification of Influenza A Viruses by GreeneChipResp Oligonucleotide Microarray, J. Clin. Microbiol., 45, 2359-2364.
[16] Rota, P. A., Oberste, M. S., Monroe, S. S., Nix, W. A., Campagnoli, R., Icenogle, J. P., Penaranda, S., Bankamp, B., Maher, K., Chen, M.-h., Tong, S., Tamin, A., Lowe, L., Frace, M., DeRisi, J. L., Chen, Q., Wang, D., Erdman, D. D., Peret, T. C. T., Burns, C., Ksiazek, T. G., Rollin, P. E., Sanchez, A., Liffick, S., Holloway, B., Limor, J., McCaustland, K., Olsen-Rasmussen, M., Fouchier, R., Gunther, S., Osterhaus, A. D. M. E., Drosten, C., Pallansch, M. A., Anderson, L. J. and Bellini, W. J. (2003) Characterization of a Novel Coronavirus Associated with Severe Acute Respiratory Syndrome, Science, 300, 1394-1399.
[17] Satya, R., Zavaljevski, N., Kumar, K. and Reifman, J. (2008) A high-throughput pipeline for designing microarray-based pathogen diagnostic assays, BMC Bioinformatics, 9, doi: 10.1186/1471-2105-1189-1185.
[18] Sengupta, S., Onodera, K., Lai, A. and Melcher, U. (2003) Molecular Detection and Identification of Influenza Viruses by Oligonucleotide Microarray Hybridization, J. Clin. Microbiol., 41, 4542-4550.
[19] Singh-Gasson, S., Green, R., Yue, Y., Nelson, C., Blattner, F., Sussman, M. and Cerrina, F. (1999) Maskless fabrication of light-directed oligonucleotide microarrays using a digital micromirror array, Nat Biotechnol 17, 974-978.
[20] Slezak, T., Kuczmarski, T., Ott, L., Tones, C., Medeiros, D., Smith, J., Truitt, B., Mulakken, N., Lam, M., Vitalis, E., Zemla, A., Zhou, C. E. and Gardner, S. (2003) Comparative genomics tools applied to bioterrorism defense, Briefings in Bioinformatics, 4, 133-149.
[21] Urisman, A., Molinaro, R. J., Fischer, N., Plummer, S. J., Casey, G., Klein, E. A., Malathi, K., Magi-Galluzzi, C., Tubbs, R. R., Ganem, D., Silverman, R. H. and DeRisi, J. L. (2006)

Identification of a Novel Gammaretrovirus in Prostate Tumors of Patients Homozygous for R462Q<italic>RNASEL</italic> Variant, PLoS Pathog, 2, e25.
[22] Wang, D., Coscoy, L., Zylberberg, M., Avila, P. C., Boushey, H. A., Ganem, D. and DeRisi, J. L. (2002) Microarray-based detection and genotyping of viral pathogens, Proceedings of the National Academy of Sciences of the United States of America, 99, 15687-15692.
[23] Wang, D., Urisman, A., Liu, Y., Springer, M., Ksiazek, T., Erdman, D., Mardis, E., Hickenbotham, M., Magrini, V., Eldred, J., Latreille, J., Wilson, R., Ganem, D. and DeRisi, J. (2003) Viral Discovery and Sequence Recovery Using DNA Microarrays, PLoS Biol., 1, e2.
[24] Wang, X.-W., Zhang, L., Jin, L.-Q., Jin, M., Shen, Z.-Q., An, S., Chao, F.-H. and Li, J.-W. (2007) Development and application of an oligonucleotide microarray for the detection of food-borne bacterial pathogens, Applied Microbiology and Biotechnology, 76, 225-233.
[25] Wong, C., Heng, C., Wan Yee, L., Soh, S., Kartasasmita, C., Simoes, E., Hibberd, M., Sung, W.-K. and Miller, L. (2007) Optimization and clinical validation of a pathogen detection microarray, Genome Biology, 8, R93.
[26] Li, W. and Godzik, A. (2006) Cd-hit: a fast program for clustering and comparing large sets of protein or nucleotide sequences. Bioinformatics, 22, 1658-1659.
[27] SantaLucia, J. and Hicks, D. (2004) The thermodynamics of DNA strucutural motifs. Ann. Rev. Biophys. Biomol. Struct., (33):415-440.
[28] Gardner S N, Jaing C J, McLoughlin K S, Slezak T. A microbial detection array (MDA) for viral and bacterial detection. 2010. BMC Genomics, 11:668.
[29] Victoria, J. G., Wang, C., Jones, M. S., Jaing, C., McLoughlin, K., Gardner, S., and Delwart, E. L. 2010. Viral nucleic acids in live-attenuated vaccines: detection of minority variants and an adventitious virus. Journal of Virology, 84(12) doi:10.1128/JVI.02690-09
[30] Erlandsson L, Rosenstierne M W, McLoughlin K, Jaing C, Formsgaard A 2011. The Microbial Detection Array Combined with Random Phi29-Amplification Used as a Diagnostic fool for Virus Detection in Clinical Samples. PLoS ONE 6(8): e22631. doi: 10.1371/journal.pone.
[31] McLoughlin, Kevin S. “Microarrays for pathogen detection and analysis.” Briefings in functional genomics 10.6 (2011): 342-353.
[32] Jaing, Crystal, et al. “Detection of Adventitious Viruses from Biologicals Using a Broad-Spectrum Microbial Detection Array,” PDA Journal of Pharmaceutical Science and Technology 65.6 (2011)-668-674.
[33] Hysom, David A., et al. “Skip the alignment: degenerate, multiplex primer and probe design using K-mer matching, instead of alignments.” PLoS One 7.4 (2012): e34560,

What is claimed is:

1. A computer-based method to obtain a plurality of oligonucleotide probes for detection of targets of a target group comprising the following computer-operated steps wherein a computer performs the steps in single-processor mode or multiple-processor mode:
providing an initial genomic collection;
identifying group-specific candidate probes from the initial genomic collection by eliminating from the initial collection regions with matches to non-group targets above a match threshold and by selecting regions satisfying probe characteristics, said probe characteristics including at least one criterion selected from length, Tₘ, GC %, maximum homopolymer length, homodimer free energy prediction, hairpin free energy prediction, probe-target free energy prediction, and minimum trimer frequency entropy condition;
ranking the group-specific candidate probes in decreasing order of number of targets of the target group represented by each group-specific candidate probe; and
selecting probes from the ranked group-specific candidate probes, thus obtaining the plurality of oligonucleotide probes for detection of targets of a target group, wherein a target is represented if a candidate probe matches with at least 85% sequence similarity over the total candidate probe length and has a perfectly matching subsequence of at least 29 contiguous bases spanning the middle of the probe.

2. A computer-based method to obtain a plurality of oligonucleotide probes for detection of targets of a target group comprising the following computer-operated steps wherein a computer performs the steps in single-processor mode or multiple-processor mode:
providing an initial genomic collection;
identifying group-specific candidate probes from the initial genomic collection by eliminating from the initial collection regions with matches to non-group targets above a match threshold and by selecting regions satisfying probe characteristics, said probe characteristics including at least one criterion selected from length, Tₘ, GC %, maximum homopolymer length, homodimer free energy prediction, hairpin free energy prediction, probe-target free energy prediction, and minimum trimer frequency entropy condition;
ranking the group-specific candidate probes in decreasing order of number of targets of the target group represented by each group-specific candidate probe;
selecting probes from the ranked group-specific candidate probes;
thus obtaining the plurality of oligonucleotide probes for detection of targets of a target group, wherein a target is represented if a candidate probe matches an at least 85% sequence identity to the target over the length of the probe and a detection probability of at least 85% derived from an alignment score, a predicted Tₘ, and the start position of the match on the probe.

3. The method of claim 2, wherein selecting probes from the ranked group-specific candidate probes comprises, for each target, selecting the most conserved or least conserved probes representing that target until each target genome is represented by a predetermined number of probes.

4. The method of claim 2, further comprising clustering together candidate probes sharing at least 90% identity and selecting one candidate probe from each cluster.

5. The method of claim 2, wherein the at least one criterion is relaxed to obtain at least a minimum number of candidate probes for each target.

6. The method of claim 2, wherein the group is selected between a viral family, a bacterial family, a viral sequence group classified under a taxonomic node other than family, a bacterial sequence group classified under a taxonomic node other than family, a fungal group, a protozoan group, or an archaeal group.

7. The method of claim 2, wherein the probes are at least 30 per target.

8. The method of claim 7, wherein the probes are at least 30 conserved probes and at least 5 discriminating probes.

9. The method of claim 2, wherein the probes are at least 40 bases long.

10. The method of claim 2, wherein group-specific regions are identified for probe selection that do not have a match of an oligonucleotide of x or more nucleotides long with sequences not part of the group, x being an integer.

11. The method of claim 10, wherein x is 19, 20, 21, or 22 nucleotides for a group.

12. The method of claim 2, wherein the alignment score is a BLAST bit score.

13. A method to obtain and synthesize a plurality of oligonucleotide probes for detection of targets of a target group, comprising:
performing the method of claim 2; and
synthesizing the obtained plurality of oligonucleotide probes for detection of targets of a target group.

14. A plurality of oligonucleotide probes for detection of targets of a target group, the plurality obtained with the method of claim 13.

15. An array comprising the plurality of oligonucleotide probes according to claim 14.

16. The array of claim 14, wherein the number of probes of the array differs according to the target.

17. A computer-based method to obtain a plurality of oligonucleotide probes for detection of targets of a target group comprising the following computer-operated steps wherein a computer performs the steps in single-processor mode or multiple-processor mode:
providing an initial genomic collection;
identifying group-specific candidate probes from the initial genomic collection by k-mer analysis, wherein k-mer analysis comprises:
compiling sequences of targets independent of any alignment,
enumerating all k-mers of a desired probelength range of the compiled sequences, wherein k is the desired number of bases in a family-unique region,
ranking k-mers by the number of target sequences in which they occur,
picking conserved k-mers from the ranked k-mers,
filtering conserved k-mers for desired characteristics,
aligning filtered conserved k-mers to targets,
recording detected targets from the alignment as probes, wherein the recording is iterated to find another k-mer for remaining targets,
aligning probes against target sequences, and
selecting probes from the matches of the alignments that satisfy at least a minimum desired oligo length, thus obtaining the plurality of oligonucleotide probes for detection of targets of a target group.

18. The method of claim 17, wherein the desired characteristics include length of a probe, homopolymer length, trimer entropy, Tₘ, hairpin avoidance, and/or GC %.

19. The method of claim 17, wherein aligning filtered conserved k-mers to targets further comprises recalculating conservation to allow mismatches.

20. The method of claim 19, wherein the mismatches are degenerate bases thus providing degenerate probes.

21. The method of claim 20, further comprising calculating degenerate probes, wherein a degenerate probe comprises up to a maximum number of degenerate bases.

22. The method of claim 21, wherein the maximum number of degenerate bases is no more than 6 bases.

23. The method of claim 22, further comprises replacing degenerate bases with the most common non-degenerate base for each degenerate base position after aligning probes against target sequences.

24. The method of claim 15, wherein aligning against target sequencing is performed by BLAST.

25. A method to obtain and synthesize a plurality of oligonucleotide probes for detection of targets of a target group, comprising:
performing the method of claim 17; and
synthesizing the obtained plurality of oligonucleotide probes for detection of targets of a target group.

26. A plurality of oligonucleotide probes for detection of targets of a target group, the plurality obtained with the method of claim 25.

27. An array comprising the plurality of oligonucleotide probes according to claim 26.

28. The array of claim 27, wherein the number of probes of the array differs according to the target.
